SEQUENCE LISTING (2 sequences)

SEQ ID NO: 1 - protein, 214 aa, synthetic construct
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 449 aa, synthetic construct
QVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2026/0069688 A1
(43) Pub. Date: Mar. 12, 2026

(54) STABLE ANTIBODY COMPOSITION

(71) Applicant: CHONG KUN DANG PHARMACEUTICAL CORP., Seoul (KR)

(72) Inventors: Yoon Seok LEE, Yongin-si, Gyeonggi-do (KR); Sang Oh KWON, Yongin-si, Gyeonggi-do (KR); Mi Gyeong KIM, Yongin-si, Gyeonggi-do (KR); Hee Hun YOON, Yongin-si, Gyeonggi-do (KR); Sa Muk LEE, Yongin-si, Gyeonggi-do (KR); So Hyun KIM, Yongin-si, Gyeonggi-do (KR)

(21) Appl. No.: 19/106,898

(22) PCT Filed: Oct. 19, 2023
(86) PCT No.: PCT/KR2023/016295

§ 371 Date: Feb. 26, 2025

(30) Foreign Application Priority Data
Oct. 21, 2022 (KR) 10-2022-0136845

(51) Int. Cl. A61K 39/395; A61K 47/02; A61K 47/10; A61K 47/12; A61K 47/18; A61K 47/22; A61K 47/26; C07K 16/24

(52) U.S. Cl. CPC A61K 39/39591; A61K 47/02; A61K 47/10; A61K 47/12; A61K 47/183; A61K 47/22; A61K 47/26; C07K 16/244; C07K 2317/24

(57) ABSTRACT

The present disclosure relates to an aqueous pharmaceutical composition, a method of preparing the same, and use thereof, the aqueous pharmaceutical composition including: risankizumab or an antigen-binding fragment thereof; and a stabilizer, wherein the aqueous pharmaceutical composition does not include a polyol.

### TECHNICAL FIELD

The present disclosure relates to a stable pharmaceutical composition including risankizumab or an antigen-binding fragment thereof, a method of preparing the composition, and medical use of the composition for treating various diseases.

### BACKGROUND ART

Risankizumab is a highly efficient and specific inhibitor of interleukin-23 (IL-23), and is a humanized immunoglobulin G1 (IgG1) monoclonal antibody directed against a p19 subunit of IL-23. Risankizumab, due to its binding to IL-23 p19, inhibits the action of IL-23 to induce and maintain T helper (Th) 17 cells, innate lymphoid cells, γδT cells, and natural killer (NK) cells, which are responsible for tissue inflammation, destruction, and abnormal tissue restoration. Risankizumab is known to be effective in the treatment of autoimmune diseases, specifically inflammatory bowel disease (e.g. Crohn's disease and ulcerative colitis), multiple sclerosis, rheumatoid arthritis, ankylosing spondylitis, psoriatic arthritis, or psoriasis (see WO2012/061448).

For effective administration of risankizumab, a pharmaceutical formulation with excellent stability is required. WO2021/048743 discloses a liquid pharmaceutical formulation comprising risankizumab together with a polyol as a stabilizer and a surfactant. However, polyols represented by sugars or sugar alcohols are known to contain monosaccharides as impurities, and it is also known that polyols can decompose into monosaccharides depending on pH or temperature stress conditions (see The effect of sucrose hydrolysis on the stability of protein therapeutics during accelerated formulation studies. Journal of pharmaceutical sciences, 2009 December; 98(12):4501-10). However, it is known that monosaccharides can react with amino acid groups of proteins and cause glycation, which has a potential risk of causing denaturation of proteins (aggregates, etc.) or affecting the efficacy of proteins (see Glycation of polyclonal IgGs: Effect of sugar excipients during stability studies, European Journal of Pharmaceutics and Biopharmaceutics Volume 102, May 2016, Pages 185-190; Quantitative analysis of glycation and its impact on antigen binding, MAbs. 2018, Volume 10, No. 3, 406-415).

### PRIOR ART DOCUMENTS

### Patent Documents

Patent Document 1: WO2012/061448
Patent Document 2: WO2021/048743

### Non-Patent Documents

Non-Patent Document 1: The effect of sucrose hydrolysis on the stability of protein therapeutics during accelerated formulation studies. Journal of pharmaceutical sciences, 2009 December; 98(12):4501-10
Non-Patent Document 2: Quantitative analysis of glycation and its impact on antigen binding, MAbs. 2018, Volume 10, No. 3, 406-415

### DISCLOSURE

### Technical Problem

Therefore, there is a need for the development of a risankizumab formulation capable of further improving stability of risankizumab while including a stabilizer that can replace a polyol.

An aspect of the present disclosure is to provide a stable pharmaceutical composition including risankizumab or an antigen-binding fragment thereof, suitable for use as a medicine for the treatment of a subject.

Another aspect of the present disclosure is to provide a method of treating an autoimmune disease in a subject, including administering the pharmaceutical composition to the subject.

Another aspect of the present disclosure is to provide a method of preparing the pharmaceutical composition.

### Technical Solution

An aspect of the present disclosure provides an aqueous pharmaceutical composition including:
(a) risankizumab or an antigen-binding fragment thereof; and
(b) a stabilizer,
wherein the aqueous pharmaceutical composition does not include a polyol.

Another aspect of the present disclosure provides an aqueous pharmaceutical composition including:
(a) risankizumab or an antigen-binding fragment thereof; and
(b) an amino acid or a pharmaceutically acceptable salt thereof,
wherein (c) the aqueous pharmaceutical composition has a pH of 5.0 to 7.0.

Another aspect of the present disclosure provides a method of treating an autoimmune disease, including administering the aforementioned pharmaceutical composition to a subject.

### Advantageous Effects

An aqueous pharmaceutical composition including risankizumab or an antigen-binding fragment thereof according to an aspect was confirmed to have excellent stability under various conditions including thermal stability, photostability, freezing and/or freeze-thaw stability, and agitation stability by including, as a stabilizer, an amino acid or a salt thereof, or a metal salt, such as sodium chloride, while not including a polyol that has been conventionally used as a stabilizer. Moreover, due to excellent stability even under conditions with or without a surfactant and/or a buffer, there is an advantage of being able to avoid side effects that may occur by including a surfactant and/or a buffer.

### DESCRIPTION OF DRAWINGS

FIG. 1 shows an amino acid sequence of a light chain of risankizumab (SEQ ID NO: 1).

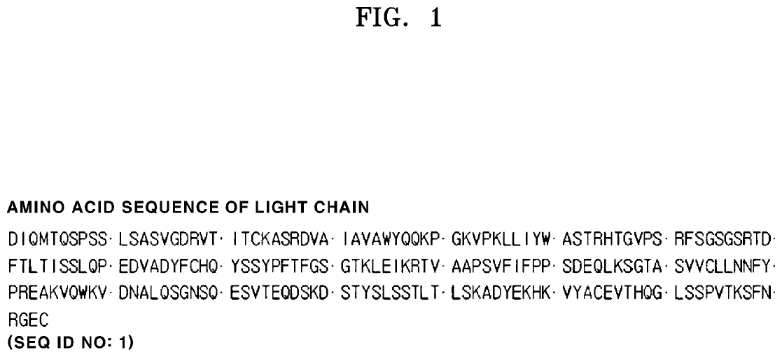

FIG. 2 shows an amino acid sequence of a heavy chain of risankizumab (SEQ ID NO: 2).

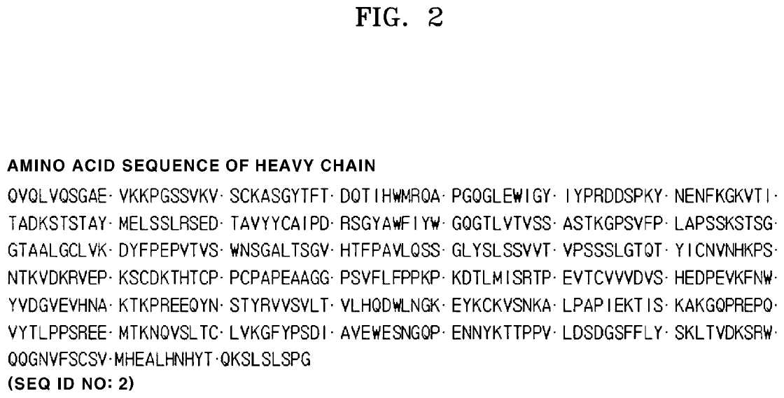

FIG. 3 is a graph of showing changes in HMW % (ΔHMW %) at Weeks 1, 2, and 4 relative to the initial period observed in a thermal stability test for a risankizumab formulation including a stabilizer only without a buffer and a surfactant.

FIG. 4 shows graphs obtained by performing stability modeling through Design of Experiments (DoE) statistical analysis on results (SE-HPLC and WCX results) of a thermal stability test for a risankizumab formulation including proline as a stabilizer by designing 5 factors (protein concentration, pH, histidine buffer concentration, proline stabilizer concentration, and surfactant concentration) based on DoE.

FIG. 5 shows graphs obtained by performing stability modeling through Design of Experiments (DoE) statistical analysis on results (SE-HPLC and WCX results) of an agitation stability test for a risankizumab formulation including proline as a stabilizer by designing 5 factors (protein concentration, pH, histidine buffer concentration, proline stabilizer concentration, and surfactant concentration) based on DoE.

FIG. 6 shows graphs obtained by performing stability modeling through Design of Experiments (DoE) statistical analysis on results (SE-HPLC, WCX, and HIC results) of a photostability test for a risankizumab formulation including proline as a stabilizer by designing 5 factors (protein concentration, pH, histidine buffer concentration, proline stabilizer concentration, and surfactant concentration) based on DoE.

FIG. 7 shows graphs obtained by performing stability modeling through Design of Experiments (DoE) statistical analysis on results (SE-HPLC and WCX results) of a freeze-thaw stability test for a risankizumab formulation including proline as a stabilizer by designing 5 factors (protein concentration, pH, histidine buffer concentration, proline stabilizer concentration, and surfactant concentration) based on DoE.

### MODE FOR THE INVENTION

All technical terms as used in the present specification have the same meaning as commonly understood by those of ordinary skill in the relevant art, unless otherwise defined. In addition, suitable methods or samples are described in the present specification, but similar or equivalent ones are also within the scope of the present specification. Also, although not explicitly stated, numerical values described in the present specification are considered to include the meaning of “about”. The contents of all publications referred in the present specification are incorporated herein by reference in their entirety.

According to an aspect of the present disclosure, an aqueous pharmaceutical composition includes:
(a) risankizumab or an antigen-binding fragment thereof; and
(b) a stabilizer,
wherein the aqueous pharmaceutical composition does not include a polyol.

The stabilizer may include an amino acid or a pharmaceutically acceptable salt thereof, or a metal salt.

The metal salt may be NaCl, KCl, NaF, KBr, NaBr, Na₂SO₄, NaSCN, CaCl₂, MgCl₂, or K₂SO₄. The metal salt may be, for example, NaCl or Na₂SO₄. The metal salt may be present at a concentration of 0.5 wt % to 1 wt % in the pharmaceutical composition. In an embodiment, the pharmaceutical composition may include 0.8 wt % of sodium chloride. The concentration of the metal salt may be freely adjusted within a range in which the stability of a risankizumab or an antigen-binding fragment thereof in the pharmaceutical composition is maintained, and may individually vary depending on each specific type of metal salts.

According to another aspect of the present disclosure, an aqueous pharmaceutical composition includes:
(a) a risankizumab or an antigen-binding fragment thereof; and
(b) an amino acid or a pharmaceutically acceptable salt thereof,
wherein (c) the aqueous pharmaceutical composition has a pH of 5.0 to 7.0. The pharmaceutical composition according to this aspect may not include a polyol.

Hereinafter, the pharmaceutical compositions according to the two aspects will be described in more detail.

The amino acid may serve as a stabilizer, and may include lysine, arginine, glycine, proline, histidine, alanine, valine, leucine, isoleucine, proline, phenylalanine, tyrosine, tryptophan, serine, threonine, cysteine, methionine, asparagine, glutamine, aspartic acid, glutamic acid, or a pharmaceutically acceptable salt of the foregoing, or a mixture of the foregoing, but is not limited thereto In an embodiment, the amino acid may be lysine, arginine, glycine, proline, histidine, a pharmaceutically acceptable salt of the foregoing, or any combination of the foregoing. In an embodiment, the amino acid may be proline. The concentration of the amino acid as the stabilizer may be 0.1 mM to 300.0 mM, 0.5 mM to 300.0 mM, 1.0 mM to 300.0 mM, 5.0 mM to 300.0 mM, 10.0 mM to 300.0 mM, 25.0 mM to 300.0 mM, 30.0 mM to 300.0 mM, 50.0 mM to 300.0 mM, 80.0 mM to 300.0 mM, 100.0 mM to 300.0 mM, 120.0 mM to 300.0 mM, 0.1 mM to 250.0 mM, 0.5 mM to 250.0 mM, 1.0 mM to 250.0 mM, 5.0 mM to 250.0 mM, 10.0 mM to 250.0 mM, 25.0 mM to 250.0 mM, 30.0 mM to 250.0 mM, 50.0 mM to 250.0 mM, 80.0 mM to 250.0 mM, 100.0 mM to 250.0 mM, 120.0 mM to 250.0 mM, 0.1 mM to 200.0 mM, 0.5 mM to 200.0 mM, 1.0 mM to 200.0 mM, 5.0 mM to 200.0 mM, 10.0 mM to 200.0 mM, 25.0 mM to 200.0 mM, 30.0 mM to 200.0 mM, 50.0 mM to 200.0 mM, 80.0 mM to 200.0 mM, 100.0 mM to 200.0 mM, 120.0 mM to 200.0 mM, 0.1 mM to 160.0 mM, 0.5 mM to 160.0 mM, 1.0 mM to 160.0 mM, 5.0 mM to 160.0 mM, 10.0 mM to 160.0 mM, 25.0 mM to 160.0 mM, 30.0 mM to 160.0 mM, 50.0 mM to 160.0 mM, 80.0 mM to 160.0 mM, 100.0 mM to 160.0 mM, 120.0 mM to 160.0 mM, 130.0 mM to 150.0 mM, 0.1 mM to 100.0 mM, 0.5 mM to 100.0 mM, 1.0 mM to 100.0 mM, 5.0 mM to 100.0 mM, 10.0 mM to 100.0 mM, 25.0 mM to 100.0 mM, 30.0 mM to 100.0 mM, 50.0 mM to 100.0 mM, 80.0 mM to 100.0 mM, 0.1 mM to 50.0 mM, 0.5 mM to 50.0 mM, 1.0 mM to 50.0 mM, 5.0 mM to 50.0 mM, 10.0 mM to 50.0 mM, 25.0 mM to 50.0 mM, 30.0 mM to 50.0 mM, 0.1 mM to 40.0 mM, 0.5 mM to 40.0 mM, 1.0 mM to 40.0 mM, 5.0 mM to 40.0 mM, 10.0 mM to 40.0 mM, 25.0 mM to 40.0 mM, 30.0 mM to 40.0 mM, 0.1 mM to 30.0 mM, 0.5 mM to 30.0 mM, 1.0 mM to 30.0 mM, 5.0 mM to 30.0 mM, 10.0 mM to 30.0 mM, 25.0 mM to 30.0 mM, 0.1 mM to 20.0 mM, 0.5 mM to 20.0 mM, 1.0 mM to 20.0 mM, 5.0 mM to 20.0 mM, 10.0 mM to 20.0 mM, 0.1 mM to 10.0 mM, 0.5 mM to 10.0 mM, 1.0 mM to 10.0 mM, or 5.0 mM to 10.0 mM. Alternatively, the concentration of the amino acid may be 1 wt % to 3 wt %. In an embodiment, the amino acid may be 100 mM to 200 mM histidine, specifically 150 mM histidine, a pharmaceutically acceptable salt thereof, or a mixture of the foregoing. In an embodiment, the amino acid may be lysine, arginine, glycine, or proline, at a concentration of 1 wt % to 3 wt %. The concentration of the amino acid may be adjusted within a range in which the stability of the risankizumab or the antigen-binding fragment thereof can be obtained without affecting a desirable pH of the pharmaceutical composition, and may vary depending on specific amino acids. In an embodiment, the amino acid may be 2.5 wt % proline.

A polyol not included in the pharmaceutical composition may be, for example, sorbitol, sucrose, trehalose, mannose, maltose, mannitol, or a mixture of the foregoing. The pharmaceutical composition may have excellent stability by including, as a stabilizer, a metal salt or an amino acid, while not including a polyol (see Test Example 3, Formulations 5 to 9, 14 to 18, 20, and 22; and Test Example 4, Formulations 5 to 8). This is an unexpected effect when considering that risankizumab-containing pharmaceutical formulations in the art generally include a polyol for stability. In addition, a polyol may include monosaccharides as impurities, and monosaccharides are known to have a potential risk of causing denaturation (aggregates, etc.) of risankizumab or affecting efficacy of proteins (see Non-Patent Documents 1 and 2). In this regard, the pharmaceutical composition may avoid the potential risk of destabilization of protein active ingredients by monosaccharides due to the absence of a polyol.

Also, the pharmaceutical composition may or may not include a surfactant. Excellent stability may be achieved with or without a surfactant (see Test Example 3, Formulations 5 to 9 and 20 vs. Formulations 14 to 18 and 22; Test Example 4, Formulations 5 to 8).

Also, the pharmaceutical composition may or may not include a buffer. Excellent stability may be achieved with or without a buffer (see Test Example 3, Formulations 5 to 9 and 20 vs. Formulations 14 to 18 and 22; Test Example 4, Formulations 5 to 8).

In an embodiment, the pharmaceutical composition may include neither a polyol nor a surfactant.

The expression “the pharmaceutical composition does not include an ingredient A” as used in the present specification may refer that the pharmaceutical formulation does not, or substantially not include, the ingredient A. The expression “substantially not include the ingredient A” may be interpreted to encompass a case where the ingredient A is not resent at all, a case where the ingredient A is present in a trace amount, if any, so as not to substantially affect features of the pharmaceutical composition, or a case where the ingredient A is present in an undetectable amount.

The surfactant may be, for example, polysorbate, poloxamer, a sorbitan ester of another fatty acid, or a mixture of the foregoing. The polysorbate may be, for example, polysorbate 20, polysorbate 80, or a mixture of the foregoing. In an embodiment, the surfactant may be polysorbate 20. The concentration of the surfactant in the pharmaceutical composition may be 0.001 wt % to 2 wt % in the pharmaceutical composition, and may vary depending on specific types of surfactants. In an embodiment, the surfactant may be 0.02 wt % polysorbate 20.

The pH of the pharmaceutical composition may be 5.0 to 7.0. In detail, for example, the pH of the pharmaceutical composition may be pH 5 to 6.8, pH 5 to 6.5, pH 5 to 6.3, pH 5.2 to 6.3, pH 5.0 to 6.0, pH 5.2 to 6.0, pH 5.0 to 5.8, pH 5.2 to 5.8, pH 5.0 to 5.6, pH 5.2 to 5.6, pH 5.1, pH 5.2, pH 5.3, pH 5.4, pH 5.5, pH 5.7, or pH 6.0. In an embodiment, the pH of the pharmaceutical composition may be 5.7.

The pharmaceutical composition may include a buffer. The buffer may be, for example, acetate, succinate, citrate, glutamate, glycine, lactate, maleate, phosphate, tartrate, or histidine buffer, or any combination of the foregoing, but is not limited thereto. The concentration of the buffer in the pharmaceutical composition may be included in a suitable amount for maintaining the selected pH during a storage period of the pharmaceutical composition. For example, the buffer may be present at a concentration of 10 mM to 60 mM in the pharmaceutical composition, and the concentration of the buffer may vary depending on specific types of buffers. In an embodiment, the buffer may be 16 mM histidine. The “risankizumab” which is the main ingredient included in the pharmaceutical composition is currently marketed under the trade name SKYRIZI® as an antibody used for the treatment of autoimmune diseases, especially psoriasis or psoriatic arthritis. The risankizumab is known to be used in treating autoimmune diseases, inflammatory diseases, respiratory diseases, metabolic disorders or cancer, more specifically psoriasis, psoriatic arthritis, inflammatory bowel disease, multiple sclerosis, rheumatoid arthritis, Crohn's disease, ulcerative colitis, ankylosing spondylitis, asthma, chronic obstructive pulmonary disease (COPD), or the like. The risankizumab may also include “biosimilars” or “biobetters” of active risankizumab antibodies present in commercially available SKYRIZI®.

The risankizumab may be an antibody including a light chain having the amino acid sequence of SEQ ID NO: 1 and a heavy chain having the amino acid sequence of SEQ ID NO: 2.

The term “antigen-binding fragment” as used in the present specification refers to a fragment capable of binding to the target antigen, IL-23 p19, of the antibody, i.e. the risankizumab, including, for example, Fab fragment, F(ab′)2 fragment, Fc fragment, or scFv fragment, but is not limited thereto.

The risankizumab may be produced by general methods known in the art. For example, WO2012/061448 discloses methods that a person skilled in the art can use to prepare the risankizumab. These methods are incorporated herein by reference.

The concentration of the risankizumab or the antigen-binding fragment thereof in the pharmaceutical composition may be, for example, 9 mg/ml to 170 mg/mL, 9 mg/mL to 45 mg/mL, 10 mg/mL to 40 mg/mL, 15 mg/mL to 35 mg/mL, 20 mg/mL to 30 mg/mL, 130 mg/mL to 160 mg/mL, or about 150 mg/mL.

The pharmaceutical composition may be an aqueous liquid formulation.

The pharmaceutical composition may be administered by parenteral delivery. Parenteral administration may include, for example, not only subcutaneous, intramuscular, intradermal, and intramedullary injections, but also intrathecal, direct intracerebroventricular, intravenous, intraperitoneal, and intravitreal injections. Drugs may be administered in a variety of conventional ways, such as intraperitoneal, parenteral, intraarterial, or intravenous injection.

The pharmaceutical composition may be for subcutaneous injection, intramuscular injection, or intravenous injection. The pharmaceutical composition may further include an aqueous carrier suitable for injection. The aqueous carrier may be a safe, non-toxic, pharmaceutically acceptable carrier when administered to a human, and examples thereof may include water, a saline solution, a Ringer's solution, dextrose, or a mixture of the foregoing. In an embodiment, the aqueous carrier may be water.

The pharmaceutical composition may have an osmotic pressure being in an appropriate range for subcutaneous or intravenous injection. The osmotic pressure may be, for example, 200 mOsm/kg to 400 mOsm/kg, 200 mOsm/kg to 350 mOsm/kg, 250 mOsm/kg to 300 mOsm/kg, 250 mOsm/kg to 290 mOsm/kg, 270 mOsm/kg to 328 mOsm/kg, 250 mOsm/kg to 269 mOsm/kg, or 328 mOsm/kg to 350 mOsm/kg. The osmotic pressure may be appropriately adjusted to minimize a pain that may be caused during administration.

In an embodiment, in the pharmaceutical composition, the concentration of the risankizumab or the antigen-binding fragment thereof may be 9 mg/mL to 170 mg/mL, the stabilizer may be sodium chloride, lysine, arginine, glycine, proline, histidine, or a pharmaceutically acceptable salt of the foregoing, or a mixture of the foregoing, or sodium chloride, the surfactant may be polysorbate 20, the pH may be 5.0 to 7.0, and the polyol may not be included.

In an embodiment, in the pharmaceutical composition, the concentration of the risankizumab or the antigen-binding fragment thereof may be 9 mg/mL to 170 mg/mL, the stabilizer may be sodium chloride, lysine, arginine, glycine, proline, histidine, or a pharmaceutically acceptable salt thereof, or a mixture of the foregoing, or sodium chloride, the pH may be 5.0 to 7.0, and the polyol and the surfactant may not be included.

In an embodiment, the pharmaceutical composition may include 150 mg/mL risankizumab, 16 mM histidine, 2.5 wt % proline, and 0.02 wt % polysorbate 20, have a pH of 5.7, and optionally not include the polyol and/or the surfactant.

In an embodiment, the pharmaceutical composition may include 150 mg/mL risankizumab, 10 mM histidine, 2.5 wt % proline, and 0.02 wt % polysorbate 20, have a pH of 5.7, and optionally not include the polyol and/or the surfactant.

In the pharmaceutical composition of the present disclosure, the risankizumab or the antigen-binding fragment thereof may be stabilized. The term “stabilization” refers that the risankizumab or the antigen-binding fragment thereof substantially retains its physical stability, chemical stability, and/or biological activity before and after administration, during additional manufacturing processes, preservation, or storage. The physical stability, chemical stability, and/or biological activity may be evaluated by commonly known methods. In an embodiment, the stability may be evaluated by testing thermal stability, photostability, freeze-thaw stability, and agitation stability, as shown in Examples below.

The risankizumab may be used for treatments known to be effective for any disease in the art. For example, WO2012/061448 discloses a list of indications that can be treated by administering the risankizumab. These methods are incorporated herein by reference. Therefore, another aspect of the present disclosure provides a method of treating an autoimmune disease, such as psoriasis or psoriatic arthritis, the method including administering the aforementioned pharmaceutical composition to a subject. In addition to the aforementioned diseases, inflammatory diseases, respiratory diseases, metabolic disorders or cancer, more specifically, inflammatory bowel diseases (Crohn's disease, ulcerative colitis), multiple sclerosis, rheumatoid arthritis, ankylosing spondylitis, asthma, chronic obstructive pulmonary disease (COPD), or the like may be treated. The method of treatment may include administering a therapeutically effective amount of a pharmaceutical formulation to a subject. The subject may include a human.

Another aspect of the present invention provides the aforementioned pharmaceutical composition for treatment of autoimmune diseases, cancer, psoriasis, psoriatic arthritis, inflammatory bowel disease, multiple sclerosis, rheumatoid arthritis, ankylosing spondylitis, asthma, chronic obstructive pulmonary disease (COPD), or the like.

Another aspect of the present invention includes a method of preparing the aforementioned pharmaceutical composition, the method including: preparing a mixed solution by adding a stabilizer to an aqueous carrier; and adding risankizumab or an antigen-binding fragment thereof to the mixed solution; or
the method including: preparing a solution by adding risankizumab or an antigen-binding fragment thereof to an aqueous carrier; and adding a stabilizer to the solution,
wherein the method is performed optionally without adding a polyol.

The aqueous carrier may be an aqueous solvent (e.g., water or a saline solution).

In the method, a buffer (or a buffer solution) and/or a surfactant may be optionally added at the step of adding the stabilizer.

Details of the method of preparing the pharmaceutical composition may be directly applied from the description of the pharmaceutical composition according to an aspect of the present disclosure.

Hereinafter, the technical features and effects of the present disclosure will be described in more detail through Examples and Experimental examples. However, the following Examples and Experimental examples are provided only for illustrative purposes to aid understanding of the present disclosure, and the scope and ranges of the present disclosure are not limited thereto.

### Explanation of Abbreviations

| Abbreviation | Explanation |
|---|---|
| SE-HPLC | Size exclusion-high-performance liquid |
| | chromatography |
| WCX | Weak cation exchange chromatography |
| HIC | Hydrophobic interaction |
| | chromatography |
| HMW % | High molecular weight |
| LMW % | Low molecular weight |
| Monomer % | Monomer content |
| Acidic % | Acidic variant percentage |
| Basic % | Basic variant percentage |
| Main % | Main percentage |
| Hydrophobic % | Hydrophobic variant percentage |
| Hydrophilic % | Hydrophilic variant percentage |
| Rpm | Revolutions per minute |

### Analysis Methods

In analysis methods of the following Test Examples, SE-HPLC and WCX were performed on samples to determine thermal stability, freeze-thaw stability, and agitation stability. By SE-HPLC analysis, HMW %, LMW %, and Monomer % were measured. By WCX analysis, Acidic %, Basic %, and Main % were measured.

Also, HIC as well as SE-HPLC and WCX was performed to determine photostability. By HIC, Hydrophobic %, Hydrophilic %, and Main % were measured.

In the SE-HPLC analysis, a high-performance liquid chromatography (HPLC) system, such as the Alliance HPLC system from Waters (Milford, MA, USA), which includes a size exclusion chromatography (SEC) column is used. Proteins separated from the SEC column can be detected by UV absorption at 280 nm, and determination of relative amounts can be performed by calculating area under the curve (AUC) for each separated peak. Peaks can be assigned to various species according to separation times corresponding to the molecular size of species. To determine relative HMW %, relative LMW %, and relative Monomer % of antibodies, especially monomeric antibodies, in the formulation, HMW and LMW species are separated from each other when present in the formulation. In particular, a relative content or a relative amount is expressed as a percentage value, and the sum of Monomer %, HMW % and LMW % is 100%.

In the weak cation exchange chromatography (WCX), a high-performance liquid chromatography (HPLC) system, such as the Alliance HPLC system from Waters (Milford, MA, USA), which includes a WCX column is used. Proteins separated from the WCX column can be detected by UV absorption at 280 nm, and determination of relative amounts can be performed by calculating area under the curve (AUC) for each separated peak or each group of separated peaks. Peaks can be assigned to various species according to separation conditions corresponding to the surface charge of antibody species. In the case of stability analysis, the measurement can be performed after the preparation of formulations (T0) and subsequently after the indicated storage time under the stated storage conditions. Acidic % or Basic % includes all peaks before or after the Main % original antibody variants peak. These peaks represent inclusion of antibody variants with more acidity and/or more basicity compared to the Main % original antibody variants, and/or with more negative or positive charges on their surface under chromatographic conditions. In particular, the relative content or the relative amount is expressed as a percentage value, and the sum of Main %, Acidic %, and Basic % is 100%. In the HIC analysis, a high-performance liquid chromatography (HPLC) system, such as the Alliance HPLC system from Waters (Milford, MA, USA), which includes a HIC column, is used. Proteins separated from the HIC column can be detected by UV absorption at 280 nm, and determination of relative amounts can be performed by calculating area under the curve (AUC) for each separated peak or each group of separated peaks. Peaks can be assigned to various species according to separation times corresponding to the hydrophobicity of species. To determine relative Hydrophobic %, relative Hydrophilic %, and relative Main % of antibodies, hydrophobic and hydrophilic species are separated from each other when present in the formulation. In particular, the relative content or the relative amount is expressed as a percentage value, and the sum of Hydrophobic %, Hydrophilic %, and Main % is 100%.

### Test Example 1: Stability Analysis of Formulation Depending on pH and Buffer Solution

Aqueous risankizumab liquid formulations of compositions shown in Table 1 below were prepared, and then analyzed for thermal stability, photostability, freeze-thaw stability, and agitation stability.

TABLE 1
Formulation compositions depending on pH and buffer solution

| | Protein | | | | |
|---|---|---|---|---|---|
| | concen- | | | | |
| | tration of | | | Stabi- | Surfac- |
| Formulation | risankizumab | Buffer solution | pH | lizer | tant |
| Formulation 1 | 150 mg/mL | 10 mM acetate | 5.7 | 7% | 0.02% |
| Formulation 2 | | N/A | 5.7 | treha- | polysor- |
| Formulation 3 | | 10 mM acetate | 4.0 | lose | bate 20 |
| Formulation 4 | | 10 mM acetate | 5.0 | | |
| Formulation 5 | | 10 mM acetate | 6.0 | | |
| Formulation 6 | | 10 mM citrate | 4.0 | | |
| Formulation 7 | | 10 mM citrate | 5.0 | | |
| Formulation 8 | | 10 mM citrate | 6.0 | | |
| Formulation 9 | | 10 mM succinate | 4.0 | | |
| Formulation 10 | | 10 mM succinate | 5.0 | | |
| Formulation 11 | | 10 mM succinate | 6.0 | | |
| Formulation 12 | | 10 mM histidine | 5.0 | | |
| Formulation 13 | | 10 mM histidine | 6.0 | | |
| Formulation 14 | | 10 mM histidine | 7.0 | | |
| Formulation 15 | | 10 mM phosphate | 6.0 | | |
| Formulation 16 | | 10 mM phosphate | 7.0 | | |
| Formulation 17 | | 10 mM phosphate | 8.0 | | |

Preparation of samples: Formulation samples were collected after storage under various stress conditions to determine stability thereof. Dialysis was performed by using each prepared buffer solution, and formulation samples were prepared in a desired protein concentration. The prepared formulation was sterile filtered, filled with 1 mL in a syringe, and exposed to various stress conditions. The stress conditions are as follows. For the thermal stability, the samples were stored and collected at Weeks 1, 2, 4, and 5 under temperature conditions of 40±2° C. For the photostability, the samples were stored and collected under conditions of an illuminance of not less than 1.2 million lux hours and an integrated near ultraviolet of not less than 200 watt hours/square meter. For the freeze-thaw stability, the samples were collected after repeating a freeze-thaw cycle 5 times under conditions of room temperature and a temperature of −60° C. or lower. For the agitation stability, the samples were collected after stirring under conditions of 0 rpm and 300 rpm.

Results: Results of measuring the thermal stability, photostability, freeze-thaw stability, and agitation stability are shown in Tables 2 to 28 below.

TABLE 2
SE-HPLC HMW (%) measurement results (thermal stability)

| | HMW (%) | | | | |
|---|---|---|---|---|---|
| Formulation | Initial | Week 1 | Week 2 | Week 4 | Week 5 |
| Formulation 1 | 0.90 | 1.73 | 2.05 | 2.59 | 2.76 |
| Formulation 2 | 1.35 | 2.04 | 2.37 | 2.91 | 3.16 |
| Formulation 3 | 1.86 | 3.97 | 3.70 | 4.85 | 5.96 |
| Formulation 4 | 0.65 | 1.45 | 1.77 | 2.29 | 2.66 |
| Formulation 5 | 1.08 | 2.04 | 2.36 | 2.90 | 3.12 |
| Formulation 6 | 2.08 | 15.95 | 7.54 | 5.50 | 6.59 |
| Formulation 7 | 1.24 | 2.29 | 2.49 | 3.08 | 3.51 |
| Formulation 8 | 1.01 | 1.99 | 2.29 | 2.80 | 3.20 |
| Formulation 9 | 2.24 | 5.82 | 4.50 | 5.25 | 6.57 |
| Formulation 10 | 0.67 | 1.43 | 1.74 | 2.38 | 2.87 |
| Formulation 11 | 1.11 | 2.06 | 2.46 | 3.03 | 3.29 |
| Formulation 12 | 0.70 | 1.32 | 1.58 | 2.03 | 2.33 |
| Formulation 13 | 1.05 | 1.56 | 1.93 | 2.30 | 2.56 |
| Formulation 14 | 1.56 | 2.26 | 2.76 | 3.35 | 3.68 |
| Formulation 15 | 1.25 | 2.30 | 2.74 | 3.30 | 3.79 |
| Formulation 16 | 1.77 | 3.11 | 3.67 | 4.76 | 5.33 |
| Formulation 17 | 2.92 | 4.70 | 5.58 | 6.76 | 7.91 |

TABLE 3
SE-HPLC LMW (%) measurement results (thermal stability)

| | LMW (%) | | | | |
|---|---|---|---|---|---|
| Formulation | Initial | Week 1 | Week 2 | Week 4 | Week 5 |
| Formulation 1 | 1.89 | 2.72 | 2.89 | 4.17 | 4.40 |
| Formulation 2 | 2.17 | 2.59 | 2.61 | 3.87 | 4.04 |
| Formulation 3 | 2.11 | 2.49 | 2.57 | 3.58 | 3.87 |
| Formulation 4 | 2.00 | 4.33 | 6.13 | 9.38 | 10.50 |
| Formulation 5 | 2.05 | 2.50 | 2.58 | 3.52 | 3.69 |
| Formulation 6 | 2.05 | 5.41 | 9.03 | 14.20 | 16.30 |
| Formulation 7 | 2.05 | 3.14 | 4.08 | 5.82 | 6.46 |
| Formulation 8 | 2.08 | 2.46 | 2.52 | 3.57 | 3.86 |
| Formulation 9 | 2.34 | 4.47 | 6.74 | 10.38 | 11.84 |
| Formulation 10 | 2.28 | 2.94 | 3.63 | 5.18 | 5.86 |
| Formulation 11 | 2.10 | 2.39 | 2.65 | 3.48 | 3.71 |
| Formulation 12 | 2.07 | 2.59 | 2.97 | 4.40 | 4.89 |
| Formulation 13 | 2.32 | 2.41 | 2.53 | 3.48 | 3.89 |
| Formulation 14 | 2.19 | 2.53 | 2.77 | 4.11 | 4.30 |
| Formulation 15 | 2.10 | 2.52 | 2.66 | 3.66 | 3.83 |
| Formulation 16 | 1.90 | 2.54 | 2.77 | 4.15 | 4.60 |
| Formulation 17 | 2.15 | 2.94 | 3.76 | 5.46 | 6.30 |

TABLE 4
SE-HPLC Monomer (%) measurement results (thermal stability)

| | Monomer (%) | | | | |
|---|---|---|---|---|---|
| Formulation | Initial | Week 1 | Week 2 | Week 4 | Week 5 |
| Formulation 1 | 96.93 | 95.68 | 95.35 | 93.54 | 93.20 |
| Formulation 2 | 96.54 | 95.48 | 95.06 | 93.51 | 92.98 |
| Formulation 3 | 96.14 | 91.70 | 90.16 | 85.77 | 83.55 |
| Formulation 4 | 97.47 | 95.82 | 95.34 | 93.54 | 92.94 |
| Formulation 5 | 96.87 | 95.46 | 95.06 | 93.57 | 93.19 |
| Formulation 6 | 95.88 | 78.64 | 83.43 | 80.30 | 77.11 |
| Formulation 7 | 96.71 | 94.57 | 93.43 | 91.11 | 90.03 |
| Formulation 8 | 96.92 | 95.55 | 95.19 | 93.63 | 92.93 |
| Formulation 9 | 95.43 | 89.72 | 88.76 | 84.37 | 81.58 |
| Formulation 10 | 97.05 | 95.63 | 94.64 | 92.45 | 91.28 |
| Formulation 11 | 96.79 | 95.54 | 94.89 | 93.50 | 93.00 |
| Formulation 12 | 97.22 | 96.10 | 95.45 | 93.57 | 92.78 |
| Formulation 13 | 96.63 | 96.03 | 95.54 | 94.22 | 93.55 |
| Formulation 14 | 96.25 | 95.21 | 94.47 | 92.55 | 92.03 |
| Formulation 15 | 96.64 | 95.18 | 94.59 | 93.05 | 92.37 |
| Formulation 16 | 96.33 | 94.35 | 93.56 | 91.09 | 90.07 |
| Formulation 17 | 94.94 | 92.35 | 90.67 | 87.78 | 85.80 |

The results determined by the SE-HPLC analysis at a storage temperature of 40° C. are shown in Tables 2 to 4.

During the 5-week storage period, the HMW % increased by 2% to 5%. The greatest increase was measured to be +4.10%, +4.51%, +4.33%, and +4.99% for Formulation 3, Formulation 6, Formulation 9, and Formulation 17, respectively, and the increase for the rest of the formulations except for the formulations with the greatest increase was measured to be +2.17% in average.

During the 5-week storage period, the LMW % increased by 2% to 14%. The greatest increase was measured to be +8.50%, +14.25%, +9.50%, and +4.15% for Formulation 4, Formulation 6, Formulation 9, and Formulation 17, respectively, and the increase for the rest of the formulations except for the formulations with the greatest increase was measured to be +2.35% in average.

During the 5-week storage period, the Monomer % decreased by 3% to 19%. The greatest decrease was measured to be −12.59%, −18.77%, −13.85%, and −9.14% for Formulation 3, Formulation 6, Formulation 9, and Formulation 17, respectively, and the decrease for the rest of the formulations except for the formulations with the greatest decrease was measured to be −4.52% in average.

TABLE 5
WCX Acidic (%) measurement results (thermal stability)

| | Acidic (%) | | | | |
|---|---|---|---|---|---|
| Formulation | Initial | Week 1 | Week 2 | Week 4 | Week 5 |
| Formulation 1 | 17.57 | 22.02 | 28.06 | 36.53 | 41.43 |
| Formulation 2 | 17.74 | 20.77 | 27.12 | 35.22 | 39.04 |
| Formulation 3 | 17.81 | 29.55 | 17.10 | 17.16 | 17.95 |
| Formulation 4 | 19.62 | 22.81 | 27.49 | 34.97 | 40.52 |
| Formulation 5 | 18.69 | 26.06 | 27.71 | 35.50 | 40.58 |
| Formulation 6 | 16.90 | 21.30 | 22.98 | 22.95 | 22.65 |
| Formulation 7 | 17.55 | 20.75 | 31.07 | 38.66 | 42.11 |
| Formulation 8 | 17.84 | 21.01 | 28.27 | 37.18 | 42.28 |
| Formulation 9 | 17.21 | 22.78 | 22.57 | 22.55 | 21.97 |
| Formulation 10 | 17.54 | 21.16 | 28.85 | 35.92 | 40.40 |
| Formulation 11 | 17.22 | 21.85 | 28.05 | 35.94 | 39.88 |
| Formulation 12 | 17.13 | 26.98 | 24.86 | 30.56 | 34.28 |
| Formulation 13 | 18.75 | 22.17 | 26.23 | 32.75 | 37.65 |
| Formulation 14 | 18.55 | 21.65 | 31.52 | 42.70 | 48.66 |
| Formulation 15 | 18.35 | 21.66 | 28.09 | 36.10 | 43.40 |
| Formulation 16 | 17.33 | 16.50 | 33.83 | 46.57 | 53.36 |
| Formulation 17 | 18.73 | 21.47 | 57.33 | 45.97 | 54.98 |

TABLE 6
WCX Basic (%) measurement results (thermal stability)

| | Basic (%) | | | | |
|---|---|---|---|---|---|
| Formulation | Initial | Week 1 | Week 2 | Week 4 | Week 5 |
| Formulation 1 | 7.48 | 9.89 | 12.57 | 13.41 | 13.47 |
| Formulation 2 | 7.61 | 6.45 | 12.36 | 12.38 | 12.93 |
| Formulation 3 | 9.26 | 8.23 | 41.96 | 54.77 | 57.76 |
| Formulation 4 | 8.65 | 9.23 | 17.23 | 19.95 | 20.15 |
| Formulation 5 | 7.87 | 8.68 | 11.13 | 11.59 | 10.87 |
| Formulation 6 | 8.98 | 8.80 | 47.68 | 61.75 | 64.70 |
| Formulation 7 | 9.41 | 14.07 | 25.17 | 29.55 | 32.07 |
| Formulation 8 | 8.26 | 9.80 | 12.00 | 11.94 | 11.87 |
| Formulation 9 | 8.31 | 17.53 | 43.24 | 55.89 | 62.89 |
| Formulation 10 | 8.02 | 28.52 | 23.33 | 27.92 | 29.29 |
| Formulation 11 | 7.22 | 9.09 | 11.42 | 11.88 | 12.36 |
| Formulation 12 | 7.82 | 19.15 | 18.79 | 22.14 | 23.64 |
| Formulation 13 | 7.02 | 31.06 | 11.39 | 12.18 | 11.98 |
| Formulation 14 | 7.46 | 9.33 | 9.87 | 9.17 | 8.71 |
| Formulation 15 | 7.84 | 13.41 | 11.77 | 12.53 | 11.17 |
| Formulation 16 | 9.04 | 28.26 | 11.13 | 10.74 | 10.31 |
| Formulation 17 | 8.05 | 9.95 | 10.90 | 29.22 | 23.50 |

TABLE 7
WCX Main (%) measurement results (thermal stability)

| | Main (%) | | | | |
|---|---|---|---|---|---|
| Formulation | Initial | Week 1 | Week 2 | Week 4 | Week 5 |
| Formulation 1 | 74.95 | 68.09 | 59.36 | 50.05 | 45.10 |
| Formulation 2 | 74.65 | 50.77 | 60.52 | 52.40 | 48.03 |
| Formulation 3 | 72.94 | 62.21 | 40.94 | 28.07 | 24.29 |
| Formulation 4 | 71.73 | 67.96 | 55.27 | 45.08 | 39.34 |
| Formulation 5 | 73.44 | 65.26 | 61.17 | 52.91 | 48.55 |
| Formulation 6 | 74.12 | 69.90 | 29.34 | 15.30 | 12.65 |
| Formulation 7 | 73.04 | 65.18 | 43.75 | 31.79 | 25.82 |
| Formulation 8 | 73.90 | 69.20 | 59.73 | 50.88 | 45.85 |
| Formulation 9 | 74.48 | 59.68 | 34.20 | 21.56 | 15.13 |
| Formulation 10 | 74.44 | 50.31 | 47.82 | 36.16 | 30.31 |
| Formulation 11 | 75.56 | 69.06 | 60.53 | 52.17 | 47.77 |
| Formulation 12 | 75.06 | 53.87 | 56.35 | 47.30 | 42.09 |
| Formulation 13 | 74.23 | 46.77 | 62.38 | 55.07 | 50.37 |
| Formulation 14 | 73.99 | 69.02 | 58.61 | 48.13 | 42.63 |
| Formulation 15 | 73.81 | 64.94 | 60.14 | 51.36 | 45.43 |
| Formulation 16 | 73.63 | 55.24 | 55.04 | 42.69 | 36.33 |
| Formulation 17 | 73.22 | 68.58 | 31.77 | 24.81 | 21.51 |

The results determined by the WCX analysis at a storage temperature of 40° C. are shown in Tables 5 to 7.

During the 5-week storage period, the Acidic % increased by 0% to 36%. The smallest increase was measured to be +0.14%, +5.75%, and +4.76% for Formulation 3, Formulation 6, and Formulation 9, respectively, and the increase for the rest of the formulations except for the formulations with the smallest increase was measured to be +24.78% in average.

During the 5-week storage period, the Basic % increased by 1% to 56%. The greatest increase was measured to be +48.50%, +55.72%, +54.58%, and +15.45% for Formulation 3, Formulation 6, Formulation 9, and Formulation 17, respectively, and the increase for the rest of the formulations except for the formulations with the greatest increase was measured to be +8.26% in average.

During the 5-week storage period, the Main % decreased by 24% to 62%. The greatest decrease was measured to be −48.65%, −61.47%, −59.35%, and −51.71% for Formulation 3, Formulation 6, Formulation 9, and Formulation 17, respectively, and the decrease for the rest of the formulations except for the formulations with the greatest decrease was measured to be −36.96% in average.

TABLE 8
SE-HPLC HMW (%) measurement results (photostability)

HMW (%)

| Formulation | Initial | Dark condition | Light exposure |
|---|---|---|---|
| Formulation 1 | 0.90 | 0.93 | 5.46 |
| Formulation 2 | 1.35 | 1.26 | 8.03 |
| Formulation 3 | 1.86 | 1.64 | 2.99 |
| Formulation 4 | 0.65 | 0.79 | 2.98 |
| Formulation 5 | 1.08 | 1.22 | 9.61 |
| Formulation 6 | 2.08 | 1.89 | 3.88 |
| Formulation 7 | 1.24 | 1.36 | 5.03 |
| Formulation 8 | 1.01 | 1.28 | 6.90 |
| Formulation 9 | 2.24 | 1.93 | 3.37 |
| Formulation 10 | 0.67 | 0.72 | 2.64 |
| Formulation 11 | 1.11 | 1.24 | 8.98 |
| Formulation 12 | 0.70 | 0.73 | 3.08 |
| Formulation 13 | 1.05 | 0.94 | 3.97 |
| Formulation 14 | 1.56 | 1.53 | 5.95 |
| Formulation 15 | 1.25 | 1.34 | 7.50 |
| Formulation 16 | 1.77 | 2.03 | 13.41 |
| Formulation 17 | 2.92 | 2.90 | 16.57 |

TABLE 9
SE-HPLC LMW (%) measurement results (photostability)

LMW (%)

| Formulation | Initial | Dark condition | Light exposure |
|---|---|---|---|
| Formulation 1 | 1.89 | 1.75 | 1.99 |
| Formulation 2 | 2.17 | 1.72 | 2.00 |
| Formulation 3 | 2.11 | 2.03 | 2.25 |
| Formulation 4 | 2.00 | 1.82 | 2.14 |
| Formulation 5 | 2.05 | 1.69 | 2.09 |
| Formulation 6 | 2.05 | 2.13 | 2.71 |
| Formulation 7 | 2.05 | 1.85 | 2.57 |
| Formulation 8 | 2.08 | 1.74 | 2.06 |
| Formulation 9 | 2.34 | 2.05 | 2.26 |
| Formulation 10 | 2.28 | 1.86 | 2.21 |
| Formulation 11 | 2.10 | 2.05 | 2.07 |
| Formulation 12 | 2.07 | 1.80 | 2.12 |
| Formulation 13 | 2.32 | 1.78 | 1.98 |
| Formulation 14 | 2.19 | 1.77 | 1.87 |
| Formulation 15 | 2.10 | 1.75 | 2.01 |
| Formulation 16 | 1.90 | 1.73 | 1.95 |
| Formulation 17 | 2.15 | 1.71 | 1.87 |

TABLE 10
SE-HPLC Monomer (%) measurement results (photostability)

Monomer (%)

| Formulation | Initial | Dark condition | Light exposure |
|---|---|---|---|
| Formulation 1 | 96.93 | 97.32 | 92.55 |
| Formulation 2 | 96.54 | 97.01 | 89.98 |
| Formulation 3 | 96.14 | 96.33 | 94.75 |
| Formulation 4 | 97.47 | 97.40 | 94.88 |
| Formulation 5 | 96.87 | 97.09 | 88.31 |
| Formulation 6 | 95.88 | 95.98 | 93.41 |
| Formulation 7 | 96.71 | 96.79 | 92.40 |
| Formulation 8 | 96.92 | 96.98 | 91.03 |
| Formulation 9 | 95.43 | 96.02 | 94.37 |
| Formulation 10 | 97.05 | 97.42 | 95.14 |
| Formulation 11 | 96.79 | 96.71 | 88.95 |
| Formulation 12 | 97.22 | 97.48 | 94.80 |
| Formulation 13 | 96.63 | 97.28 | 94.05 |
| Formulation 14 | 96.25 | 96.70 | 92.17 |
| Formulation 15 | 96.64 | 96.91 | 90.49 |
| Formulation 16 | 96.33 | 96.24 | 84.64 |
| Formulation 17 | 94.94 | 95.39 | 81.57 |

The results determined by the SE-HPLC analysis under conditions of an illuminance of not less than 1.2 million lux hours and an integrated near ultraviolet of not less than 200 watt hours/square meter are shown in Tables 8 to 10.

After light exposure, the HMW % increased by 1% to 14%. The greatest increase was measured to be +11.38% and +13.67% for Formulation 16 and Formulation 17, respectively, and the increase for the rest of the formulations except for the formulations with the greatest increase was measured to be +4.08% in average.

After light exposure, the LMW % showed no significant increase, and was measured to be +0.29% in average.

After light exposure, the Monomer % decreased by 2% to 14%. The greatest decrease was measured to be −11.60% and −13.82% for Formulation 16 and Formulation 17, respectively, and the decrease for the rest of the formulations except for the formulations with the greatest decrease was measured to be −4.38% in average.

TABLE 11
WCX Acidic (%) measurement results (photostability)

Acidic (%)

| Formulation | Initial | Dark condition | Light exposure |
|---|---|---|---|
| Formulation 1 | 17.57 | 19.16 | 23.96 |
| Formulation 2 | 17.74 | 19.05 | 25.61 |
| Formulation 3 | 17.81 | 17.99 | 16.04 |
| Formulation 4 | 19.62 | 18.88 | 20.62 |
| Formulation 5 | 18.69 | 19.12 | 27.08 |
| Formulation 6 | 16.90 | 18.44 | 18.22 |
| Formulation 7 | 17.55 | 18.74 | 21.90 |
| Formulation 8 | 17.84 | 19.17 | 26.01 |
| Formulation 9 | 17.21 | 18.28 | 16.65 |
| Formulation 10 | 17.54 | 18.84 | 18.92 |
| Formulation 11 | 17.22 | 18.86 | 27.72 |
| Formulation 12 | 17.13 | 18.36 | 19.87 |
| Formulation 13 | 18.75 | 19.03 | 25.21 |
| Formulation 14 | 18.55 | 19.44 | 30.83 |
| Formulation 15 | 18.35 | 19.52 | 25.11 |
| Formulation 16 | 17.33 | 19.90 | 30.64 |
| Formulation 17 | 18.73 | 21.82 | 34.42 |

TABLE 12
WCX Basic (%) measurement results (photostability)

Basic (%)

| Formulation | Initial | Dark condition | Light exposure |
|---|---|---|---|
| Formulation 1 | 7.48 | 8.84 | 27.05 |
| Formulation 2 | 7.61 | 8.74 | 27.44 |
| Formulation 3 | 9.26 | 11.96 | 34.69 |
| Formulation 4 | 8.65 | 9.44 | 28.22 |
| Formulation 5 | 7.87 | 8.55 | 27.00 |
| Formulation 6 | 8.98 | 12.69 | 31.90 |
| Formulation 7 | 9.41 | 10.52 | 31.03 |
| Formulation 8 | 8.26 | 9.16 | 23.62 |
| Formulation 9 | 8.31 | 12.26 | 36.30 |
| Formulation 10 | 8.02 | 10.30 | 32.21 |
| Formulation 11 | 7.22 | 8.96 | 27.33 |
| Formulation 12 | 7.82 | 9.47 | 27.56 |
| Formulation 13 | 7.02 | 8.53 | 19.34 |
| Formulation 14 | 7.46 | 8.40 | 13.90 |
| Formulation 15 | 7.84 | 8.57 | 22.24 |
| Formulation 16 | 9.04 | 8.49 | 21.72 |
| Formulation 17 | 8.05 | 9.04 | 18.16 |

TABLE 13
WCX Main (%) measurement results (photostability)

Main (%)

| Formulation | Initial | Dark condition | Light exposure |
|---|---|---|---|
| Formulation 1 | 74.95 | 71.99 | 49.00 |
| Formulation 2 | 74.65 | 72.21 | 46.95 |
| Formulation 3 | 72.94 | 70.05 | 49.27 |
| Formulation 4 | 71.73 | 71.68 | 51.16 |
| Formulation 5 | 73.44 | 72.33 | 45.91 |
| Formulation 6 | 74.12 | 68.87 | 49.88 |
| Formulation 7 | 73.04 | 70.74 | 47.07 |
| Formulation 8 | 73.90 | 71.67 | 50.37 |
| Formulation 9 | 74.48 | 69.46 | 47.05 |
| Formulation 10 | 74.44 | 70.86 | 48.86 |
| Formulation 11 | 75.56 | 72.17 | 44.95 |
| Formulation 12 | 75.06 | 72.17 | 52.56 |
| Formulation 13 | 74.23 | 72.44 | 55.44 |
| Formulation 14 | 73.99 | 72.16 | 55.27 |
| Formulation 15 | 73.81 | 71.91 | 52.65 |
| Formulation 16 | 73.63 | 71.61 | 47.64 |
| Formulation 17 | 73.22 | 69.15 | 47.41 |

The results determined by the WCX analysis under conditions of an illuminance of not less than 1.2 million lux hours and an integrated near ultraviolet of not less than 200 watt hours/square meter are shown in Tables 11 to 13.

After light exposure, the Acidic % showed no significant increase, and was measured to be +4.96% in average.

After light exposure, the Basic % showed no significant increase, and was measured to be +16.81% in average.

After light exposure, the Main % showed no significant decrease, and was measured to be −21.77% in average.

TABLE 14
HIC Hydrophobic (%) measurement results (photostability)

Hydrophobic (%)

| Formulation | Initial | Dark condition | Light exposure |
|---|---|---|---|
| Formulation 1 | 4.11 | 4.33 | 24.61 |
| Formulation 2 | 4.15 | 4.29 | 25.31 |
| Formulation 3 | 4.35 | 4.52 | 29.38 |
| Formulation 4 | 3.97 | 4.87 | 25.16 |
| Formulation 5 | 4.21 | 4.13 | 24.36 |
| Formulation 6 | 4.94 | 4.62 | 24.92 |
| Formulation 7 | 4.82 | 4.74 | 29.43 |
| Formulation 8 | 4.36 | 4.33 | 20.77 |
| Formulation 9 | 4.63 | 4.50 | 31.91 |
| Formulation 10 | 4.55 | 4.36 | 28.58 |
| Formulation 11 | 4.55 | 4.26 | 24.90 |
| Formulation 12 | 4.36 | 4.11 | 28.35 |
| Formulation 13 | 3.76 | 4.57 | 17.26 |
| Formulation 14 | 4.17 | 4.68 | 14.31 |
| Formulation 15 | 4.23 | 4.27 | 17.06 |
| Formulation 16 | 3.99 | 4.50 | 15.86 |
| Formulation 17 | 4.79 | 5.28 | 13.53 |

TABLE 15
HIC Hydrophilic (%) measurement results (photostability)

Hydrophilic (%)

| Formulation | Initial | Dark condition | Light exposure |
|---|---|---|---|
| Formulation 1 | 2.99 | 3.05 | 5.79 |
| Formulation 2 | 4.32 | 3.88 | 7.45 |
| Formulation 3 | 4.22 | 3.91 | 3.52 |
| Formulation 4 | 2.85 | 3.00 | 4.04 |
| Formulation 5 | 3.19 | 3.12 | 8.96 |
| Formulation 6 | 3.83 | 3.65 | 4.46 |
| Formulation 7 | 3.91 | 3.34 | 4.78 |
| Formulation 8 | 3.14 | 3.31 | 6.89 |
| Formulation 9 | 3.56 | 3.71 | 4.68 |
| Formulation 10 | 2.91 | 2.76 | 4.11 |
| Formulation 11 | 3.17 | 3.21 | 8.48 |
| Formulation 12 | 2.90 | 2.95 | 3.87 |
| Formulation 13 | 3.00 | 3.07 | 5.10 |
| Formulation 14 | 3.26 | 3.43 | 6.32 |
| Formulation 15 | 3.22 | 3.28 | 7.67 |
| Formulation 16 | 3.21 | 3.43 | 10.54 |
| Formulation 17 | 4.65 | 4.31 | 10.89 |

TABLE 16
HIC Main (%) measurement results (photostability)

Main (%)

| Formulation | Initial | Dark condition | Light exposure |
|---|---|---|---|
| Formulation 1 | 92.90 | 92.62 | 69.59 |
| Formulation 2 | 91.54 | 91.83 | 67.25 |
| Formulation 3 | 91.43 | 91.57 | 67.10 |
| Formulation 4 | 92.89 | 92.13 | 70.80 |
| Formulation 5 | 92.60 | 92.75 | 66.68 |
| Formulation 6 | 91.23 | 91.73 | 70.62 |
| Formulation 7 | 91.27 | 91.92 | 65.78 |
| Formulation 8 | 92.50 | 92.36 | 72.34 |
| Formulation 9 | 91.81 | 91.79 | 63.41 |
| Formulation 10 | 92.54 | 92.89 | 67.31 |
| Formulation 11 | 92.28 | 92.53 | 66.62 |
| Formulation 12 | 92.74 | 92.94 | 67.78 |
| Formulation 13 | 93.24 | 92.37 | 77.64 |
| Formulation 14 | 92.57 | 91.89 | 79.37 |
| Formulation 15 | 92.54 | 92.45 | 75.27 |
| Formulation 16 | 92.79 | 92.08 | 73.60 |
| Formulation 17 | 90.55 | 90.41 | 75.58 |

The results determined by the HIC analysis under conditions of an illuminance of not less than 1.2 million lux hours and an integrated near ultraviolet of not less than 200 watt hours/square meter are shown in Tables 14 to 16.

After light exposure, the Hydrophobic % increased by 8% to 27%. The smallest increase was measured to be +12.69%, +9.63%, +12.79%, +11.36%, and +8.25% for Formulation 13, Formulation 14, Formulation 15, Formulation 16, and Formulation 17, respectively, and the increase for the rest of the formulations except for the formulations with the smallest increase was measured to be +22.21% in average.

After light exposure, the Hydrophilic % showed no significant increase, and was measured to be +2.96% in average.

After light exposure, the Main % decreased by 15% to 28%. The smallest decrease was measured to be −14.73%, −12.52%, −17.18%, −18.48%, and −14.83% for Formulation 13, Formulation 14, Formulation 15, Formulation 16, and Formulation 17, respectively, and the decrease for the rest of the formulations except for the formulations with the smallest decrease was measured to be −24.43% in average.

TABLE 17
SE-HPLC HMW (%) measurement results (freeze-thaw stability)

HMW (%)

| Formulation | Initial | 5 freeze-thaw cycles |
|---|---|---|
| Formulation 1 | 0.90 | 0.72 |
| Formulation 2 | 1.35 | 1.07 |
| Formulation 3 | 1.86 | 1.59 |
| Formulation 4 | 0.65 | 0.61 |
| Formulation 5 | 1.08 | 0.92 |
| Formulation 6 | 2.08 | 1.61 |
| Formulation 7 | 1.24 | 1.12 |
| Formulation 8 | 1.01 | 0.95 |
| Formulation 9 | 2.24 | 1.76 |
| Formulation 10 | 0.67 | 0.56 |
| Formulation 11 | 1.11 | 0.95 |
| Formulation 12 | 0.70 | 0.57 |
| Formulation 13 | 1.05 | 0.80 |
| Formulation 14 | 1.56 | 1.30 |
| Formulation 15 | 1.25 | 1.03 |
| Formulation 16 | 1.77 | 1.59 |
| Formulation 17 | 2.92 | 2.41 |

TABLE 18
SE-HPLC LMW (%) measurement results (freeze-thaw stability)

LMW (%)

| Formulation | Initial | 5 freeze-thaw cycles |
|---|---|---|
| Formulation 1 | 1.89 | 1.67 |
| Formulation 2 | 2.17 | 1.79 |
| Formulation 3 | 2.11 | 1.87 |
| Formulation 4 | 2.00 | 1.85 |
| Formulation 5 | 2.05 | 1.70 |
| Formulation 6 | 2.05 | 1.86 |
| Formulation 7 | 2.05 | 1.79 |
| Formulation 8 | 2.08 | 1.75 |
| Formulation 9 | 2.34 | 1.72 |
| Formulation 10 | 2.28 | 1.77 |
| Formulation 11 | 2.10 | 1.67 |
| Formulation 12 | 2.07 | 1.67 |
| Formulation 13 | 2.32 | 1.66 |
| Formulation 14 | 2.19 | 1.70 |
| Formulation 15 | 2.10 | 1.71 |
| Formulation 16 | 1.90 | 1.71 |
| Formulation 17 | 2.15 | 1.63 |

TABLE 19
SE-HPLC Monomer (%) measurement
results (freeze-thaw stability)

Monomer (%)

| Formulation | Initial | 5 freeze-thaw cycles |
|---|---|---|
| Formulation 1 | 96.93 | 97.62 |
| Formulation 2 | 96.54 | 97.14 |
| Formulation 3 | 96.14 | 96.55 |
| Formulation 4 | 97.47 | 97.53 |
| Formulation 5 | 96.87 | 97.38 |
| Formulation 6 | 95.88 | 96.54 |
| Formulation 7 | 96.71 | 97.09 |
| Formulation 8 | 96.92 | 97.30 |
| Formulation 9 | 95.43 | 96.53 |
| Formulation 10 | 97.05 | 97.67 |
| Formulation 11 | 96.79 | 97.39 |
| Formulation 12 | 97.22 | 97.73 |
| Formulation 13 | 96.63 | 97.54 |
| Formulation 14 | 96.25 | 97.00 |
| Formulation 15 | 96.64 | 97.25 |
| Formulation 16 | 96.33 | 96.71 |
| Formulation 17 | 94.94 | 95.96 |

The results determined by the SE-HPLC analysis after repeating a freeze-thaw cycle 5 times under conditions of room temperature and a temperature of −60° C. or lower are shown in Tables 17 to 19.

After 5 freeze-thaw cycles, the HMW % showed no significant increase, and was measured to be −0.23% in average.

After 5 freeze-thaw cycles, the LMW % showed no significant increase, and was measured to be −0.36% in average.

After 5 freeze-thaw cycles, the Monomer % showed no significant decrease, and was measured to be +0.59% in average.

TABLE 20
WCX Acidic (%) measurement results (freeze-thaw stability)

Acidic (%)

| Formulation | Initial | 5 freeze-thaw cycles |
|---|---|---|
| Formulation 1 | 17.57 | 19.03 |
| Formulation 2 | 17.74 | 18.53 |
| Formulation 3 | 17.81 | 18.39 |
| Formulation 4 | 19.62 | 18.98 |
| Formulation 5 | 18.69 | 19.22 |
| Formulation 6 | 16.90 | 19.79 |
| Formulation 7 | 17.55 | 19.64 |
| Formulation 8 | 17.84 | 18.69 |
| Formulation 9 | 17.21 | 17.83 |
| Formulation 10 | 17.54 | 19.04 |
| Formulation 11 | 17.22 | 17.79 |
| Formulation 12 | 17.13 | 18.88 |
| Formulation 13 | 18.75 | 17.59 |
| Formulation 14 | 18.55 | 18.68 |
| Formulation 15 | 18.35 | 17.61 |
| Formulation 16 | 17.33 | 18.01 |
| Formulation 17 | 18.73 | 18.84 |

TABLE 21
WCX Basic (%) measurement results (freeze-thaw stability)

Basic (%)

| Formulation | Initial | 5 freeze-thaw cycles |
|---|---|---|
| Formulation 1 | 7.48 | 6.88 |
| Formulation 2 | 7.61 | 6.93 |
| Formulation 3 | 9.26 | 7.32 |
| Formulation 4 | 8.65 | 7.54 |
| Formulation 5 | 7.87 | 7.57 |
| Formulation 6 | 8.98 | 8.53 |
| Formulation 7 | 9.41 | 7.60 |
| Formulation 8 | 8.26 | 7.25 |
| Formulation 9 | 8.31 | 8.14 |
| Formulation 10 | 8.02 | 7.97 |
| Formulation 11 | 7.22 | 7.96 |
| Formulation 12 | 7.82 | 8.15 |
| Formulation 13 | 7.02 | 7.64 |
| Formulation 14 | 7.46 | 6.75 |
| Formulation 15 | 7.84 | 6.70 |
| Formulation 16 | 9.04 | 6.32 |
| Formulation 17 | 8.05 | 6.90 |

TABLE 22
WCX Main (%) measurement results (freeze-thaw stability)

Main (%)

| Formulation | Initial | 5 freeze-thaw cycles |
|---|---|---|
| Formulation 1 | 74.95 | 74.09 |
| Formulation 2 | 74.65 | 74.53 |
| Formulation 3 | 72.94 | 74.28 |
| Formulation 4 | 71.73 | 73.48 |
| Formulation 5 | 73.44 | 73.21 |
| Formulation 6 | 74.12 | 71.68 |
| Formulation 7 | 73.04 | 72.76 |
| Formulation 8 | 73.90 | 74.06 |
| Formulation 9 | 74.48 | 74.03 |
| Formulation 10 | 74.44 | 72.99 |
| Formulation 11 | 75.56 | 74.25 |
| Formulation 12 | 75.06 | 72.97 |
| Formulation 13 | 74.23 | 74.78 |
| Formulation 14 | 73.99 | 74.57 |
| Formulation 15 | 73.81 | 75.69 |
| Formulation 16 | 73.63 | 75.66 |
| Formulation 17 | 73.22 | 74.26 |

The results determined by the WCX analysis after repeating a freeze-thaw cycle 5 times under conditions of room temperature and a temperature of −60° C. or lower are shown in Tables 20 to 22.

After 5 freeze-thaw cycles, the Acidic % showed no significant increase, and was measured to be +0.71% in average.

After 5 freeze-thaw cycles, the Basic % showed no significant increase, and was measured to be −0.72% in average.

After 5 freeze-thaw cycles, the Main % showed no significant decrease, and was measured to be +0.01% in average.

TABLE 23
SE-HPLC HMW (%) measurement results (agitation stability)

HMW (%)

| Formulation | 0 rpm | 300 rpm |
|---|---|---|
| Formulation 1 | 0.75 | 0.88 |
| Formulation 2 | 1.11 | 1.19 |
| Formulation 3 | 1.64 | 1.69 |
| Formulation 4 | 0.65 | 0.71 |
| Formulation 5 | 0.98 | 1.09 |
| Formulation 6 | 1.80 | 1.87 |
| Formulation 7 | 1.17 | 1.29 |
| Formulation 8 | 1.03 | 1.08 |
| Formulation 9 | 1.96 | 1.94 |
| Formulation 10 | 0.59 | 0.67 |
| Formulation 11 | 1.09 | 1.17 |
| Formulation 12 | 0.61 | 0.66 |
| Formulation 13 | 0.80 | 1.35 |
| Formulation 14 | 1.34 | 0.92 |
| Formulation 15 | 1.08 | 1.29 |
| Formulation 16 | 1.69 | 1.71 |
| Formulation 17 | 2.50 | 2.81 |

TABLE 24
SE-HPLC LMW (%) measurement results (agitation stability)

LMW (%)

| Formulation | 0 rpm | 300 rpm |
|---|---|---|
| Formulation 1 | 3.34 | 2.31 |
| Formulation 2 | 2.88 | 2.00 |
| Formulation 3 | 3.06 | 2.20 |
| Formulation 4 | 3.25 | 2.54 |
| Formulation 5 | 3.18 | 2.30 |
| Formulation 6 | 2.88 | 2.24 |
| Formulation 7 | 3.78 | 2.38 |
| Formulation 8 | 3.43 | 2.67 |
| Formulation 9 | 2.58 | 2.25 |
| Formulation 10 | 3.61 | 2.26 |
| Formulation 11 | 3.00 | 2.26 |
| Formulation 12 | 3.06 | 2.23 |
| Formulation 13 | 2.95 | 2.23 |
| Formulation 14 | 2.43 | 2.35 |
| Formulation 15 | 3.27 | 1.99 |
| Formulation 16 | 2.67 | 2.13 |
| Formulation 17 | 2.47 | 1.85 |

TABLE 25
SE-HPLC Monomer (%) measurement results (agitation stability)

Monomer (%)

| Formulation | 0 rpm | 300 rpm |
|---|---|---|
| Formulation 1 | 95.91 | 96.80 |
| Formulation 2 | 96.01 | 96.81 |
| Formulation 3 | 95.30 | 96.11 |
| Formulation 4 | 96.09 | 96.76 |
| Formulation 5 | 95.85 | 96.61 |
| Formulation 6 | 95.32 | 95.89 |
| Formulation 7 | 95.05 | 96.32 |
| Formulation 8 | 95.54 | 96.24 |
| Formulation 9 | 95.46 | 95.80 |
| Formulation 10 | 95.79 | 97.07 |
| Formulation 11 | 95.90 | 96.57 |
| Formulation 12 | 96.33 | 97.10 |
| Formulation 13 | 96.26 | 96.42 |
| Formulation 14 | 96.23 | 96.73 |
| Formulation 15 | 95.65 | 96.72 |
| Formulation 16 | 95.64 | 96.16 |
| Formulation 17 | 95.02 | 95.33 |

The results determined by the SE-HPLC analysis after stirring under conditions of 300 rpm are shown in Tables 23 to 25.

After stirring, the HMW % showed no significant increase, and was measured to be +0.09% in average.

After stirring, the LMW % showed no significant increase, and was measured to be −0.79% in average.

After stirring, the Monomer % showed no significant decrease, and was measured to be +0.70% in average.

TABLE 26
WCX Acidic (%) measurement results (agitation stability)

Acidic (%)

| Formulation | 0 rpm | 300 rpm |
|---|---|---|
| Formulation 1 | 18.97 | 18.97 |
| Formulation 2 | 18.82 | 18.78 |
| Formulation 3 | 18.08 | 18.09 |
| Formulation 4 | 18.63 | 18.86 |
| Formulation 5 | 18.90 | 18.98 |
| Formulation 6 | 18.52 | 18.34 |
| Formulation 7 | 18.57 | 18.72 |
| Formulation 8 | 18.79 | 18.81 |
| Formulation 9 | 18.27 | 18.44 |
| Formulation 10 | 18.63 | 18.85 |
| Formulation 11 | 18.89 | 19.04 |
| Formulation 12 | 18.26 | 18.54 |
| Formulation 13 | 18.96 | 18.98 |
| Formulation 14 | 19.65 | 19.83 |
| Formulation 15 | 19.16 | 19.05 |
| Formulation 16 | 19.40 | 19.32 |
| Formulation 17 | 21.11 | 20.64 |

TABLE 27
WCX Basic (%) measurement results (agitation stability)

Basic (%)

| Formulation | 0 rpm | 300 rpm |
|---|---|---|
| Formulation 1 | 8.25 | 8.12 |
| Formulation 2 | 8.11 | 7.99 |
| Formulation 3 | 10.45 | 9.89 |
| Formulation 4 | 8.83 | 8.35 |
| Formulation 5 | 8.18 | 7.91 |
| Formulation 6 | 11.01 | 10.34 |
| Formulation 7 | 9.39 | 9.26 |
| Formulation 8 | 8.38 | 8.18 |
| Formulation 9 | 10.39 | 9.77 |
| Formulation 10 | 9.27 | 9.09 |
| Formulation 11 | 8.35 | 7.70 |
| Formulation 12 | 8.83 | 8.58 |
| Formulation 13 | 8.11 | 7.48 |
| Formulation 14 | 7.86 | 7.49 |
| Formulation 15 | 8.15 | 7.93 |
| Formulation 16 | 8.18 | 7.94 |
| Formulation 17 | 8.27 | 8.49 |

TABLE 28
WCX Main (%) measurement results (agitation stability)

Main (%)

| Formulation | 0 rpm | 300 rpm |
|---|---|---|
| Formulation 1 | 72.78 | 72.92 |
| Formulation 2 | 73.07 | 73.23 |
| Formulation 3 | 71.48 | 72.03 |
| Formulation 4 | 72.53 | 72.79 |
| Formulation 5 | 72.92 | 73.11 |
| Formulation 6 | 70.47 | 71.32 |
| Formulation 7 | 72.04 | 72.03 |
| Formulation 8 | 72.83 | 73.01 |
| Formulation 9 | 71.34 | 71.79 |
| Formulation 10 | 72.09 | 72.06 |
| Formulation 11 | 72.76 | 73.26 |
| Formulation 12 | 72.91 | 72.88 |
| Formulation 13 | 72.94 | 73.54 |
| Formulation 14 | 72.49 | 72.67 |
| Formulation 15 | 72.69 | 73.02 |
| Formulation 16 | 72.42 | 72.74 |
| Formulation 17 | 70.62 | 70.87 |

The results determined by the WCX analysis after stirring under conditions of 300 rpm are shown in Tables 26 to 28.

After stirring, the Acidic % showed no significant increase, and was measured to be +0.04% in average.

After stirring, the Basic % showed no significant increase, and was measured to be −0.32% in average.

After stirring, the Main % showed no significant decrease, and was measured to be +0.30% in average.

Discussion of results: According to the results in Tables 2 to 28, the formulations including various types of buffers at a pH of 5.0 to 7.0 were determined to be stable under various stress conditions (thermal, light, freeze-thaw, and stirring stress).

### Test Example 2-1: Stability Analysis of Formulation Depending on Surfactant

Aqueous risankizumab liquid formulations of compositions shown in Table 29 below were prepared, and then analyzed for thermal stability, photostability, freeze-thaw stability, and agitation stability.

TABLE 29
Formulation compositions depending on surfactant

| | Protein | | | | |
|---|---|---|---|---|---|
| | concentration of | | | | |
| Formulation | risankizumab | Buffer solution | pH | Stabilizer | Surfactant |
| Formulation 1 | 150 mg/mL | 10 mM acetate | 6.1 | 7% | 0.02% |
| | | | | trehalose | polysorbate 20 |
| Formulation 2 | | 10 mM acetate | 6.1 | | 0.02% |
| | | | | | polysorbate 80 |
| Formulation 3 | | 10 mM acetate | 6.1 | | 0.1% |
| | | | | | poloxamer 188 |
| Formulation 4 | | 10 mM acetate | 6.1 | | N/A |

Preparation of samples: Formulation samples were collected after storage under various stress conditions to determine stability thereof. Dialysis was performed by using each prepared buffer solution, and formulation samples were prepared in a desired protein concentration. The prepared formulation was sterile filtered, filled with 1 mL in a syringe, and exposed to various stress conditions. The stress conditions are as follows. For the thermal stability, the samples were stored and collected at Weeks 1, 2, 4, and 6 under temperature conditions of 40±2° C. For the photostability, the samples were stored and collected under conditions of an illuminance of not less than 1.2 million lux hours and an integrated near ultraviolet of not less than 200 watt hours/square meter. For the freeze-thaw stability, the samples were collected after repeating a freeze-thaw cycle 5 times under conditions of room temperature and a temperature of −60° C. or lower. For the agitation stability, the samples were collected after stirring under conditions of 0 rpm and 300 rpm.

Results: Results of measuring the thermal stability, photostability, agitation stability, and freeze-thaw stability are shown in Tables 30 to 56 below.

TABLE 30
SE-HPLC HMW (%) measurement results (thermal stability)

| | HMW (%) | | | | |
|---|---|---|---|---|---|
| Formulation | Initial | Week 1 | Week 2 | Week 4 | Week 6 |
| Formulation 1 | 0.94 | 1.89 | 2.26 | 2.82 | 3.12 |
| Formulation 2 | 0.90 | 1.93 | 2.28 | 2.83 | 3.20 |
| Formulation 3 | 0.92 | 1.92 | 2.29 | 2.84 | 3.18 |
| Formulation 4 | 0.94 | 1.91 | 2.24 | 2.80 | 3.24 |

TABLE 31
SE-HPLC LMW (%) measurement results (thermal stability)

| | LMW (%) | | | | |
|---|---|---|---|---|---|
| Formulation | Initial | Week 1 | Week 2 | Week 4 | Week 6 |
| Formulation 1 | 1.66 | 2.24 | 2.73 | 3.37 | 3.63 |
| Formulation 2 | 1.65 | 2.28 | 2.75 | 3.43 | 3.69 |
| Formulation 3 | 1.70 | 2.28 | 2.78 | 3.28 | 3.82 |
| Formulation 4 | 1.74 | 2.17 | 2.71 | 3.30 | 3.76 |

TABLE 32
SE-HPLC Monomer (%) measurement results (thermal stability)

| | Monomer (%) | | | | |
|---|---|---|---|---|---|
| Formulation | Initial | Week 1 | Week 2 | Week 4 | Week 6 |
| Formulation 1 | 97.40 | 95.87 | 95.01 | 93.81 | 93.24 |
| Formulation 2 | 97.44 | 95.79 | 94.97 | 93.74 | 93.11 |
| Formulation 3 | 97.39 | 95.81 | 94.94 | 93.88 | 93.00 |
| Formulation 4 | 97.32 | 95.91 | 95.06 | 93.91 | 93.00 |

The results determined by the SE-HPLC analysis at a storage temperature of 40° C. are shown in Tables 30 to 32.

During the 6-week storage period, the HMW % showed no significant increase, and was measured to be +2.26% in average.

During the 6-week storage period, the LMW % showed no significant increase, and was measured to be +2.04% in average.

During the 6-week storage period, the Monomer % showed no significant decrease, and was measured to be −4.30% in average.

TABLE 33
WCX Acidic (%) measurement results (thermal stability)

| | Acidic (%) | | | | |
|---|---|---|---|---|---|
| Formulation | Initial | Week 1 | Week 2 | Week 4 | Week 6 |
| Formulation 1 | 18.37 | 22.51 | 27.39 | 37.52 | 43.79 |
| Formulation 2 | 18.37 | 22.97 | 27.70 | 37.54 | 43.77 |
| Formulation 3 | 18.26 | 22.69 | 28.34 | 37.51 | 44.30 |
| Formulation 4 | 18.26 | 22.70 | 27.32 | 37.36 | 43.75 |

TABLE 34
WCX Basic (%) measurement results (thermal stability)

| | Basic (%) | | | | |
|---|---|---|---|---|---|
| Formulation | Initial | Week 1 | Week 2 | Week 4 | Week 6 |
| Formulation 1 | 8.23 | 10.94 | 11.04 | 10.45 | 10.34 |
| Formulation 2 | 8.76 | 10.74 | 10.98 | 10.60 | 10.56 |
| Formulation 3 | 8.94 | 10.57 | 10.64 | 10.36 | 10.30 |
| Formulation 4 | 8.78 | 10.76 | 10.67 | 10.65 | 10.55 |

TABLE 35
WCX Main (%) measurement results (thermal stability)

| | Main (%) | | | | |
|---|---|---|---|---|---|
| Formulation | Initial | Week 1 | Week 2 | Week 4 | Week 6 |
| Formulation 1 | 73.40 | 66.55 | 61.57 | 52.04 | 45.87 |
| Formulation 2 | 72.87 | 66.29 | 61.32 | 51.86 | 45.67 |
| Formulation 3 | 72.80 | 66.74 | 61.02 | 52.12 | 45.40 |
| Formulation 4 | 72.96 | 66.54 | 62.01 | 51.99 | 45.71 |

The results determined by the WCX analysis at a storage temperature of 40° C. are shown in Tables 33 to 35. During the 6-week storage period, the Acidic % showed no significant increase, and was measured to be +25.59% in average. During the 6-week storage period, the Basic % showed no significant increase, and was measured to be +1.76% in average. During the 6-week storage period, the Main % showed no significant decrease, and was measured to be −27.35% in average.

TABLE 36
SE-HPLC HMW (%) measurement results (photostability)

HMW (%)

| Formulation | Initial | Dark condition | Light exposure |
|---|---|---|---|
| Formulation 1 | 0.94 | 1.15 | 7.50 |
| Formulation 2 | 0.90 | 1.14 | 6.80 |
| Formulation 3 | 0.92 | 1.16 | 6.14 |
| Formulation 4 | 0.94 | 1.22 | 10.83 |

TABLE 37
SE-HPLC LMW (%) measurement results (photostability)

LMW (%)

| Formulation | Initial | Dark condition | Light exposure |
|---|---|---|---|
| Formulation 1 | 1.66 | 2.00 | 2.00 |
| Formulation 2 | 1.65 | 1.97 | 1.98 |
| Formulation 3 | 1.70 | 1.95 | 1.92 |
| Formulation 4 | 1.74 | 2.01 | 2.02 |

TABLE 38
SE-HPLC Monomer (%) measurement results (photostability)

Monomer (%)

| Formulation | Initial | Dark condition | Light exposure |
|---|---|---|---|
| Formulation 1 | 97.40 | 96.85 | 90.50 |
| Formulation 2 | 97.44 | 96.89 | 91.22 |
| Formulation 3 | 97.39 | 96.89 | 91.94 |
| Formulation 4 | 97.32 | 96.77 | 87.14 |

The results determined by the SE-HPLC analysis under conditions of an illuminance of not less than 1.2 million lux hours and an integrated near ultraviolet of not less than 200 watt hours/square meter are shown in Tables 36 to 38.

After light exposure, the HMW % showed no significant increase, and was measured to be +6.65% in average.

After light exposure, the LMW % showed no significant increase, and was measured to be +0.00% in average.

After stirring, the Monomer % showed no significant decrease, and was measured to be −6.65% in average.

TABLE 39
WCX Acidic (%) measurement results (photostability)

Acidic (%)

| Formulation | Initial | Dark condition | Light exposure |
|---|---|---|---|
| Formulation 1 | 18.37 | 19.08 | 25.02 |
| Formulation 2 | 18.37 | 19.16 | 23.92 |
| Formulation 3 | 18.26 | 19.17 | 23.73 |
| Formulation 4 | 18.26 | 19.29 | 27.49 |

TABLE 40
WCX Basic (%) measurement results (photostability)

Basic (%)

| Formulation | Initial | Dark condition | Light exposure |
|---|---|---|---|
| Formulation 1 | 8.23 | 8.39 | 20.71 |
| Formulation 2 | 8.76 | 8.35 | 22.10 |
| Formulation 3 | 8.94 | 8.41 | 20.80 |
| Formulation 4 | 8.78 | 8.65 | 27.86 |

TABLE 41
WCX Main(%) measurement results (photostability)

Main (%)

| Formulation | Initial | Dark condition | Light exposure |
|---|---|---|---|
| Formulation 1 | 73.40 | 72.53 | 54.27 |
| Formulation 2 | 72.87 | 72.49 | 53.97 |
| Formulation 3 | 72.80 | 72.42 | 55.47 |
| Formulation 4 | 72.96 | 72.05 | 44.65 |

The results determined by the WCX analysis under conditions of an illuminance of not less than 1.2 million lux hours and an integrated near ultraviolet of not less than 200 watt hours/square meter are shown in Tables 39 to 41.

After light exposure, the Acidic % showed no significant increase, and was measured to be +6.73% in average.

After light exposure, the Basic % showed no significant increase, and was measured to be +14.19% in average.

After light exposure, the Main % showed no significant decrease, and was measured to be −20.92% in average.

TABLE 42
HIC Hydrophobic (%) measurement results (photostability)

Hydrophobic (%)

| Formulation | Initial | Dark condition | Light exposure |
|---|---|---|---|
| Formulation 1 | 4.35 | 4.26 | 18.58 |
| Formulation 2 | 4.50 | 4.28 | 20.05 |
| Formulation 3 | 4.32 | 1.46 | 15.41 |
| Formulation 4 | 4.40 | 4.82 | 25.38 |

TABLE 43
HIC Hydrophilic (%) measurement results (photostability)

Hydrophilic (%)

| Formulation | Initial | Dark condition | Light exposure |
|---|---|---|---|
| Formulation 1 | 3.14 | 3.24 | 7.60 |
| Formulation 2 | 3.06 | 3.26 | 7.97 |
| Formulation 3 | 3.19 | 3.37 | 7.10 |
| Formulation 4 | 3.01 | 3.15 | 9.10 |

TABLE 44
HIC Main (%) measurement results (photostability)

Main (%)

| Formulation | Initial | Dark condition | Light exposure |
|---|---|---|---|
| Formulation 1 | 92.51 | 92.50 | 73.82 |
| Formulation 2 | 92.44 | 92.47 | 71.98 |
| Formulation 3 | 92.49 | 95.18 | 77.49 |
| Formulation 4 | 92.59 | 92.04 | 65.52 |

The results determined by the HIC analysis under conditions of an illuminance of not less than 1.2 million lux hours and an integrated near ultraviolet of not less than 200 watt hours/square meter are shown in Tables 42 to 44.

After light exposure, the Hydrophobic % showed no significant increase, and was measured to be +15.46% in average.

After light exposure, the Hydrophilic % showed no significant increase, and was measured to be +4.84% in average.

After light exposure, the Main % showed no significant decrease, and was measured to be −20.31% in average.

TABLE 45
SE-HPLC HMW (%) measurement results (freeze-thaw stability)

HMW (%)

| Formulation | Initial | 5 freeze-thaw cycles |
|---|---|---|
| Formulation 1 | 0.94 | 0.95 |
| Formulation 2 | 0.90 | 0.96 |
| Formulation 3 | 0.92 | 0.92 |
| Formulation 4 | 0.94 | 0.97 |

TABLE 46
SE-HPLC LMW (%) measurement results (freeze-thaw stability)

LMW (%)

| Formulation | Initial | 5 freeze-thaw cycles |
|---|---|---|
| Formulation 1 | 1.66 | 1.87 |
| Formulation 2 | 1.65 | 1.74 |
| Formulation 3 | 1.70 | 1.47 |
| Formulation 4 | 1.74 | 1.80 |

TABLE 47
SE-HPLC Monomer (%) measurement
results (freeze-thaw stability)

Monomer (%)

| Formulation | Initial | 5 freeze-thaw cycles |
|---|---|---|
| Formulation 1 | 97.40 | 97.18 |
| Formulation 2 | 97.44 | 97.30 |
| Formulation 3 | 97.39 | 97.61 |
| Formulation 4 | 97.32 | 97.23 |

The results determined by the SE-HPLC analysis after repeating a freeze-thaw cycle 5 times under conditions of room temperature and a temperature of −60° C. or lower are shown in Tables 45 to 47.

After 5 freeze-thaw cycles, the HMW % showed no significant increase, and was measured to be +0.03% in average.

After 5 freeze-thaw cycles, the LMW % showed no significant increase, and was measured to be +0.03% in average.

After 5 freeze-thaw cycles, the Monomer % showed no significant decrease, and was measured to be −0.06% in average.

TABLE 48
WCX Acidic (%) measurement results (freeze-thaw stability)

Acidic (%)

| Formulation | Initial | 5 freeze-thaw cycles |
|---|---|---|
| Formulation 1 | 18.37 | 18.90 |
| Formulation 2 | 18.37 | 19.06 |
| Formulation 3 | 18.26 | 19.27 |
| Formulation 4 | 18.26 | 18.97 |

TABLE 49
WCX Basic (%) measurement results (freeze-thaw stability)

Basic (%)

| Formulation | Initial | 5 freeze-thaw cycles |
|---|---|---|
| Formulation 1 | 8.23 | 9.19 |
| Formulation 2 | 8.76 | 8.45 |
| Formulation 3 | 8.94 | 8.24 |
| Formulation 4 | 8.78 | 8.48 |

TABLE 50
WCX Main (%) measurement results (freeze-thaw stability)

Main (%)

| Formulation | Initial | 5 freeze-thaw cycles |
|---|---|---|
| Formulation 1 | 73.40 | 71.91 |
| Formulation 2 | 72.87 | 72.49 |
| Formulation 3 | 72.80 | 72.50 |
| Formulation 4 | 72.96 | 72.56 |

The results determined by the WCX analysis after repeating a freeze-thaw cycle 5 times under conditions of room temperature and a temperature of −60° C. or lower are shown in Tables 48 to 50.

After 5 freeze-thaw cycles, the Acidic % showed no significant increase, and was measured to be +0.73% in average.

After 5 freeze-thaw cycles, the Basic % showed no significant increase, and was measured to be −0.09% in average.

After 5 freeze-thaw cycles, the Main % showed no significant decrease, and was measured to be −0.64% in average.

TABLE 51
SE-HPLC HMW (%) measurement results (agitation stability)

HMW (%)

| Formulation | 0 rpm | 300 rpm |
|---|---|---|
| Formulation 1 | 1.05 | 1.04 |
| Formulation 2 | 1.07 | 1.02 |
| Formulation 3 | 1.05 | 0.99 |
| Formulation 4 | 1.05 | 1.06 |

TABLE 52
SE-HPLC LMW (%) measurement results (agitation stability)

LMW (%)

| Formulation | 0 rpm | 300 rpm |
|---|---|---|
| Formulation 1 | 1.90 | 1.65 |
| Formulation 2 | 1.66 | 1.71 |
| Formulation 3 | 1.72 | 1.69 |
| Formulation 4 | 1.97 | 1.63 |

TABLE 53
SE-HPLC Monomer (%) measurement results (agitation stability)

Monomer (%)

| Formulation | 0 rpm | 300 rpm |
|---|---|---|
| Formulation 1 | 97.05 | 97.31 |
| Formulation 2 | 97.27 | 97.27 |
| Formulation 3 | 97.23 | 97.32 |
| Formulation 4 | 96.99 | 97.31 |

The results determined by the SE-HPLC analysis after stirring under conditions of 300 rpm are shown in Tables 51 to 53.

After stirring, the HMW % showed no significant increase, and was measured to be −0.02% in average.

After stirring, the LMW % showed no significant increase, and was measured to be −0.14% in average.

After stirring, the Monomer % showed no significant decrease, and was measured to be +0.17% in average.

TABLE 54
WCX Acidic (%) measurement results (agitation stability)

Acidic (%)

| Formulation | 0 rpm | 300 rpm |
|---|---|---|
| Formulation 1 | 19.10 | 18.91 |
| Formulation 2 | 18.94 | 19.01 |
| Formulation 3 | 18.93 | 18.97 |
| Formulation 4 | 18.93 | 18.87 |

TABLE 55
WCX Basic (%) measurement results (agitation stability)

Basic (%)

| Formulation | 0 rpm | 300 rpm |
|---|---|---|
| Formulation 1 | 8.53 | 8.70 |
| Formulation 2 | 8.63 | 8.65 |
| Formulation 3 | 8.54 | 8.58 |
| Formulation 4 | 8.76 | 8.60 |

TABLE 56
WCX Main (%) measurement results (agitation stability)

Main (%)

| Formulation | 0 rpm | 300 rpm |
|---|---|---|
| Formulation 1 | 72.37 | 72.39 |
| Formulation 2 | 72.42 | 72.34 |
| Formulation 3 | 72.54 | 72.45 |
| Formulation 4 | 72.31 | 72.54 |

The results determined by the WCX analysis after stirring under conditions of 300 rpm are shown in Tables 54 to 56.

After stirring, the Acidic % showed no significant increase, and was measured to be −0.04% in average.

After stirring, the Basic % showed no significant increase, and was measured to be +0.02% in average.

After stirring, the Main % showed no significant decrease, and was measured to be +0.02% in average.

Discussion of results: According to the results in Tables 30 to 56, the formulations with or without a surfactant all showed stability under various stress conditions (thermal, light, freeze-thaw, and stirring stress).

### Test Example 2-2: Stability Analysis of Formulation Depending on Surfactant

Aqueous risankizumab liquid formulations of compositions shown in Table 57 below were prepared, and then analyzed for thermal stability.

TABLE 57
Formulation compositions depending on surfactant

| | Protein | | | | |
|---|---|---|---|---|---|
| | concentration of | Buffer | | | |
| Formulation | risankizumab | solution | pH | Stabilizer | Surfactant |
| Formulation 1 | 150 mg/mL | 16 mM | 5.7 | 2.5% | 0.02% polysorbate 20 |
| Formulation 2 | | histidine | | proline | 0.02% polysorbate 80 |
| Formulation 3 | | | | | 0.1% poloxamer 188 |
| Formulation 4 | | | | | N/A |

Preparation of samples: Formulation samples were collected after storage under various stress conditions to determine stability thereof. Dialysis was performed by using each prepared buffer solution, and formulation samples were prepared in a desired protein concentration. The prepared formulation was sterile filtered and 1 mL was filled into a syringe to confirm its thermal stability. For the thermal stability, the samples were stored and collected at Weeks 1, 2, and 4 under temperature conditions of 40±2° C.

Results: Results of measuring the thermal stability are shown in Tables 58 to 63 below.

TABLE 58
SE-HPLC HMW (%) measurement results (thermal stability)

HMW (%)

| Formulation | Initial | Week 1 | Week 2 | Week 4 |
|---|---|---|---|---|
| Formulation 1 | 0.78 | 1.82 | 1.41 | 1.89 |
| Formulation 2 | 0.79 | 1.17 | 1.42 | 1.88 |
| Formulation 3 | 0.83 | 1.21 | 1.42 | 2.08 |
| Formulation 4 | 0.77 | 1.16 | 1.40 | 1.87 |

TABLE 59
SE-HPLC LMW (%) measurement results (thermal stability)

LMW (%)

| Formulation | Initial | Week 1 | Week 2 | Week 4 |
|---|---|---|---|---|
| Formulation 1 | 0.19 | 0.69 | 1.19 | 2.05 |
| Formulation 2 | 0.20 | 0.70 | 1.16 | 2.04 |
| Formulation 3 | 0.21 | 0.68 | 1.19 | 2.10 |
| Formulation 4 | 0.16 | 0.69 | 1.16 | 2.00 |

TABLE 60
SE-HPLC Monomer (%) measurement results (thermal stability)

Monomer (%)

| Formulation | Initial | Week 1 | Week 2 | Week 4 |
|---|---|---|---|---|
| Formulation 1 | 99.04 | 97.48 | 97.41 | 96.06 |
| Formulation 2 | 99.01 | 98.13 | 97.42 | 96.08 |
| Formulation 3 | 98.97 | 98.11 | 97.39 | 95.83 |
| Formulation 4 | 99.08 | 98.15 | 97.45 | 96.12 |

The results determined by the SE-HPLC analysis at a storage temperature of 40° C. are shown in Tables 58 to 60.

During the 4-week storage period, the HMW % showed no significant increase, and was measured to be +1.14% in average.

During the 4-week storage period, the LMW % showed no significant increase, and was measured to be +1.86% in average.

During the 4-week storage period, the Monomer % showed no significant decrease, and was measured to be −3.00% in average.

TABLE 61
WCX Acidic (%) measurement results (thermal stability)

Acidic (%)

| Formulation | Initial | Week 1 | Week 2 | Week 4 |
|---|---|---|---|---|
| Formulation 1 | 17.38 | 20.73 | 24.61 | 31.31 |
| Formulation 2 | 17.30 | 20.27 | 24.98 | 31.46 |
| Formulation 3 | 17.31 | 21.44 | 24.93 | 31.08 |
| Formulation 4 | 17.12 | 20.75 | 24.46 | 30.36 |

TABLE 62
WCX Basic (%) measurement results (thermal stability)

Basic (%)

| Formulation | Initial | Week 1 | Week 2 | Week 4 |
|---|---|---|---|---|
| Formulation 1 | 7.74 | 12.09 | 13.62 | 15.34 |
| Formulation 2 | 7.59 | 11.69 | 13.32 | 15.60 |
| Formulation 3 | 7.51 | 10.92 | 13.26 | 15.51 |
| Formulation 4 | 7.69 | 11.68 | 13.70 | 16.14 |

TABLE 63
WCX Main (%) measurement results (thermal stability)

Main (%)

| Formulation | Initial | Week 1 | Week 2 | Week 4 |
|---|---|---|---|---|
| Formulation 1 | 74.88 | 67.18 | 61.76 | 53.35 |
| Formulation 2 | 75.11 | 68.04 | 61.70 | 52.94 |
| Formulation 3 | 75.18 | 67.63 | 61.81 | 53.40 |
| Formulation 4 | 75.19 | 67.57 | 61.84 | 53.50 |

The results determined by the WCX analysis at a storage temperature of 40° C. are shown in Tables 61 to 63. During the 4-week storage period, the Acidic % showed no significant increase, and was measured to be +13.78% in average. During the 4-week storage period, the Basic % showed no significant increase, and was measured to be +8.02% in average. During the 4-week storage period, the Main % showed no significant decrease, and was measured to be −21.79% in average.

Discussion of Results: According to the results in Tables 58 to 63, the formulations with or without a surfactant all showed stability under thermal temperature conditions.

### Test Example 3: Stability Analysis of Formulation Depending on Stabilizer

Aqueous risankizumab liquid formulations of compositions shown in Table 64 below were prepared, and then analyzed for thermal stability, photostability, freeze-thaw stability, and agitation stability.

TABLE 64
Formulation compositions depending on stabilizer

| | Protein | | | | |
|---|---|---|---|---|---|
| | concentration of | Buffer | | | |
| Formulation | risankizumab | solution | pH | Stabilizer | Surfactant |
| Formulation 1 | 150 mg/mL | 10 mM | 6.0 | 4% sorbitol | 0.02% |
| Formulation 2 | | histidine | | 4% mannitol | polysorbate 20 |
| Formulation 3 | | | | 7% sucrose | |
| Formulation 4 | | | | 7% trehalose | |
| Formulation 5 | | | | 0.8% sodium chloride | |
| Formulation 6 | | | | 2.3% lysine | |
| Formulation 7 | | | | 2.6% arginine | |
| Formulation 8 | | | | 1.5% glycine | |
| Formulation 9 | | | | 2.5% proline | |
| Formulation 10 | | | | 4% sorbitol | N/A |
| Formulation 11 | | | | 4% mannitol | |
| Formulation 12 | | | | 7% sucrose | |
| Formulation 13 | | | | 7% trehalose | |
| Formulation 14 | | | | 0.8% sodium chloride | |
| Formulation 15 | | | | 2.3% lysine | |
| Formulation 16 | | | | 2.6% arginine | |
| Formulation 17 | | | | 1.5% glycine | |
| Formulation 18 | | | | 2.5% proline | |
| Formulation 19 | | | | N/A | 0.02% |
| Formulation 20 | | | | 150 mM histidine | polysorbate 20 |
| Formulation 21 | | | | N/A | N/A |
| Formulation 22 | | | | 150 mM histidine | |

Preparation of samples: Formulation samples were collected after storage under various stress conditions to determine stability thereof. Dialysis was performed by using each prepared buffer solution, and formulation samples were prepared in a desired protein concentration. The prepared formulation was sterile filtered, filled with 1 mL in a syringe, and exposed to various stress conditions. The stress conditions are as follows. For the thermal stability, the samples were stored and collected at Weeks 2, 4, and 6 under temperature conditions of 40±2° C. For the photostability, the samples were stored and collected under conditions of an illuminance of not less than 1.2 million lux hours and an integrated near ultraviolet of not less than 200 watt hours/square meter. For the freeze-thaw stability, the samples were collected after repeating a freeze-thaw cycle 5 times under conditions of room temperature and a temperature of −60° C. or lower. For the agitation stability, the samples were collected after stirring under conditions of 0 rpm and 300 rpm.

Results: Results of measuring the stability are shown in Tables 65 to 91 below.

TABLE 65
SE-HPLC HMW (%) measurement results (thermal stability)

HMW (%)

| Formulation | Initial | Week 2 | Week 4 | Week 6 |
|---|---|---|---|---|
| Formulation 1 | 1.06 | 2.00 | 2.35 | 2.86 |
| Formulation 2 | 1.08 | 1.93 | 2.38 | 2.77 |
| Formulation 3 | 1.10 | 1.75 | 2.26 | 2.78 |
| Formulation 4 | 1.08 | 1.85 | 2.27 | 2.77 |
| Formulation 5 | 1.18 | 2.00 | 2.60 | 3.07 |
| Formulation 6 | 0.90 | 1.41 | 1.86 | 2.22 |
| Formulation 7 | 0.92 | 1.33 | 1.67 | 1.96 |
| Formulation 8 | 0.95 | 1.53 | 2.11 | 2.80 |
| Formulation 9 | 1.02 | 1.50 | 1.94 | 2.24 |
| Formulation 10 | 1.09 | 1.89 | 2.36 | 2.86 |
| Formulation 11 | 1.06 | 1.86 | 2.43 | 2.80 |
| Formulation 12 | 1.02 | 1.85 | 2.40 | 2.84 |
| Formulation 13 | 1.09 | 1.85 | 2.29 | 2.74 |
| Formulation 14 | 0.97 | 1.92 | 2.32 | 2.87 |
| Formulation 15 | 0.97 | 1.54 | 1.90 | 2.33 |
| Formulation 16 | 0.97 | 1.33 | 1.75 | 2.01 |
| Formulation 17 | 1.03 | 1.78 | 2.30 | 2.93 |
| Formulation 18 | 1.08 | 1.65 | 2.01 | 2.31 |
| Formulation 19 | 1.08 | 1.97 | 2.47 | 3.05 |
| Formulation 20 | 0.80 | 1.07 | 1.44 | 1.71 |
| Formulation 21 | 1.15 | 1.89 | 2.52 | 3.06 |
| Formulation 22 | 0.87 | 1.04 | 1.42 | 1.63 |

TABLE 66
SE-HPLC LMW (%) measurement results (thermal stability)

LMW (%)

| Formulation | Initial | Week 2 | Week 4 | Week 6 |
|---|---|---|---|---|
| Formulation 1 | 1.24 | 2.46 | 3.11 | 3.82 |
| Formulation 2 | 1.29 | 2.31 | 3.02 | 3.81 |
| Formulation 3 | 1.27 | 2.26 | 3.13 | 3.97 |
| Formulation 4 | 1.20 | 2.31 | 3.19 | 3.91 |
| Formulation 5 | 1.30 | 2.37 | 3.28 | 4.08 |
| Formulation 6 | 1.24 | 2.49 | 3.41 | 4.03 |
| Formulation 7 | 1.27 | 2.42 | 3.28 | 4.24 |
| Formulation 8 | 1.33 | 2.48 | 3.28 | 4.36 |
| Formulation 9 | 1.26 | 2.34 | 3.01 | 3.92 |
| Formulation 10 | 1.30 | 2.30 | 3.21 | 3.90 |
| Formulation 11 | 1.25 | 2.37 | 3.20 | 3.83 |
| Formulation 12 | 1.33 | 2.29 | 3.14 | 3.84 |
| Formulation 13 | 1.23 | 2.27 | 3.06 | 3.75 |
| Formulation 14 | 1.25 | 2.46 | 3.50 | 4.14 |
| Formulation 15 | 1.29 | 2.36 | 3.21 | 4.09 |
| Formulation 16 | 1.38 | 2.50 | 3.19 | 4.08 |
| Formulation 17 | 1.23 | 2.48 | 3.49 | 4.31 |
| Formulation 18 | 1.17 | 2.33 | 3.01 | 3.90 |
| Formulation 19 | 1.25 | 2.35 | 3.08 | 3.86 |
| Formulation 20 | 1.32 | 2.56 | 3.85 | 4.55 |
| Formulation 21 | 1.18 | 2.25 | 3.08 | 4.04 |
| Formulation 22 | 1.34 | 2.56 | 3.50 | 4.54 |

TABLE 67
SE-HPLC Monomer (%) measurement results (thermal stability)

Monomer (%)

| Formulation | Initial | Week 2 | Week 4 | Week 6 |
|---|---|---|---|---|
| Formulation 1 | 97.70 | 95.54 | 94.54 | 93.32 |
| Formulation 2 | 97.64 | 95.75 | 94.61 | 93.41 |
| Formulation 3 | 97.64 | 95.99 | 94.61 | 93.26 |
| Formulation 4 | 97.72 | 95.84 | 94.55 | 93.32 |
| Formulation 5 | 97.51 | 95.63 | 94.12 | 92.86 |
| Formulation 6 | 97.85 | 96.10 | 94.74 | 93.76 |
| Formulation 7 | 97.81 | 96.25 | 95.05 | 93.81 |
| Formulation 8 | 97.71 | 95.99 | 94.61 | 92.85 |
| Formulation 9 | 97.71 | 96.16 | 95.05 | 93.84 |
| Formulation 10 | 97.62 | 95.81 | 94.43 | 93.24 |
| Formulation 11 | 97.69 | 95.77 | 94.36 | 93.37 |
| Formulation 12 | 97.64 | 95.86 | 94.45 | 93.32 |
| Formulation 13 | 97.68 | 95.88 | 94.65 | 93.50 |
| Formulation 14 | 97.78 | 95.62 | 94.18 | 92.99 |
| Formulation 15 | 97.74 | 96.10 | 94.89 | 93.58 |
| Formulation 16 | 97.65 | 96.17 | 95.07 | 93.91 |
| Formulation 17 | 97.74 | 95.75 | 94.21 | 92.77 |
| Formulation 18 | 97.75 | 96.02 | 94.98 | 93.79 |
| Formulation 19 | 97.68 | 95.68 | 94.45 | 93.09 |
| Formulation 20 | 97.88 | 96.37 | 94.71 | 93.73 |
| Formulation 21 | 97.66 | 95.87 | 94.40 | 92.90 |
| Formulation 22 | 97.79 | 96.39 | 95.08 | 93.83 |

The results determined by the SE-HPLC analysis at a storage temperature of 40° C. are shown in Tables 65 to 67. During the 6-week storage period, the HMW % increased by 0.8% to 2.0%, wherein an average increase of 1.75% was measured for the formulations including a surfactant and either a polyol or a salt (Formulations 1 to 5), an average increase of 1.27% was measured for the formulations including an amino acid and a surfactant (Formulations 6 to 9 and 20), an average increase of 1.78% was measured for the formulations including a polyol or a salt (Formulations 10 to 14), and an average increase of 1.26% was measured for the formulations including an amino acid (Formulations 15 to 18 and 22).

During the 6-week storage period, the LMW % showed no significant increase, and was measured to be +2.8% in average.

During the 6-week storage period, the Monomer % decreased by 3.74% to 4.97%, wherein an average decrease of 4.41% was measured for the formulations including a surfactant and either a polyol or a salt (Formulations 1 to 5), an average decrease of 4.19% was measured for the formulations including an amino acid and a surfactant (Formulations 6 to 9 and 20), an average decrease of 4.40% was measured for the formulations including a polyol or a salt (Formulations 10 to 14), and an average decrease of 4.16% was measured for the formulations including an amino acid (Formulations 15 to 18 and 22).

TABLE 68
WCX Acidic (%) measurement results (thermal stability)

Acidic (%)

| Formulation | Initial | Week 2 | Week 4 | Week 6 |
|---|---|---|---|---|
| Formulation 1 | 20.08 | 27.43 | 31.71 | 37.87 |
| Formulation 2 | 19.69 | 28.55 | 32.86 | 38.20 |
| Formulation 3 | 21.16 | 28.75 | 32.92 | 39.05 |
| Formulation 4 | 20.16 | 27.91 | 32.86 | 39.58 |
| Formulation 5 | 19.81 | 25.74 | 29.00 | 34.42 |
| Formulation 6 | 19.85 | 26.42 | 29.21 | 35.59 |
| Formulation 7 | 19.81 | 26.26 | 29.42 | 34.60 |
| Formulation 8 | 20.58 | 29.65 | 33.50 | 41.09 |
| Formulation 9 | 20.29 | 27.63 | 31.06 | 38.90 |
| Formulation 10 | 20.30 | 27.32 | 31.31 | 38.10 |
| Formulation 11 | 20.17 | 27.75 | 32.39 | 37.65 |
| Formulation 12 | 20.27 | 27.93 | 32.61 | 38.27 |
| Formulation 13 | 20.14 | 27.79 | 32.99 | 38.78 |
| Formulation 14 | 19.99 | 25.58 | 28.22 | 34.02 |
| Formulation 15 | 20.35 | 26.23 | 29.31 | 35.63 |
| Formulation 16 | 19.55 | 25.71 | 29.34 | 34.77 |
| Formulation 17 | 21.31 | 29.29 | 34.54 | 40.58 |
| Formulation 18 | 19.92 | 27.77 | 32.67 | 38.96 |
| Formulation 19 | 19.80 | 27.82 | 31.51 | 38.52 |
| Formulation 20 | 20.04 | 28.81 | 33.77 | 40.10 |
| Formulation 21 | 20.16 | 27.40 | 31.71 | 38.28 |
| Formulation 22 | 20.03 | 28.87 | 33.62 | 40.56 |

TABLE 69
WCX Basic (%) measurement results (thermal stability)

Basic (%)

| Formulation | Initial | Week 2 | Week 4 | Week 6 |
|---|---|---|---|---|
| Formulation 1 | 7.50 | 10.74 | 12.42 | 13.31 |
| Formulation 2 | 7.58 | 10.06 | 11.64 | 12.66 |
| Formulation 3 | 7.40 | 10.14 | 11.46 | 12.83 |
| Formulation 4 | 7.43 | 10.63 | 11.99 | 12.63 |
| Formulation 5 | 7.34 | 12.19 | 14.08 | 16.01 |
| Formulation 6 | 7.31 | 12.26 | 14.64 | 15.77 |
| Formulation 7 | 7.67 | 11.36 | 13.14 | 15.36 |
| Formulation 8 | 7.13 | 10.30 | 11.24 | 12.83 |
| Formulation 9 | 7.42 | 10.29 | 11.60 | 12.58 |
| Formulation 10 | 7.35 | 10.39 | 12.17 | 12.69 |
| Formulation 11 | 7.28 | 10.55 | 11.92 | 13.53 |
| Formulation 12 | 7.36 | 10.53 | 11.64 | 13.14 |
| Formulation 13 | 7.48 | 10.58 | 11.62 | 12.53 |
| Formulation 14 | 7.51 | 13.06 | 16.00 | 18.36 |
| Formulation 15 | 7.48 | 11.85 | 14.33 | 14.59 |
| Formulation 16 | 7.44 | 11.59 | 13.32 | 14.01 |
| Formulation 17 | 7.01 | 10.25 | 11.95 | 13.06 |
| Formulation 18 | 7.52 | 10.41 | 11.72 | 12.85 |
| Formulation 19 | 7.40 | 10.69 | 12.37 | 12.60 |
| Formulation 20 | 7.17 | 10.92 | 12.49 | 14.29 |
| Formulation 21 | 7.52 | 10.83 | 12.24 | 13.05 |
| Formulation 22 | 7.25 | 11.17 | 12.61 | 14.92 |

TABLE 70
WCX Main (%) measurement results (thermal stability)

Main (%)

| Formulation | Initial | Week 2 | Week 4 | Week 6 |
|---|---|---|---|---|
| Formulation 1 | 72.42 | 61.83 | 55.87 | 48.82 |
| Formulation 2 | 72.72 | 61.39 | 55.49 | 49.13 |
| Formulation 3 | 71.44 | 61.12 | 55.63 | 48.12 |
| Formulation 4 | 72.41 | 61.46 | 55.14 | 47.79 |
| Formulation 5 | 72.85 | 62.07 | 56.91 | 49.57 |
| Formulation 6 | 72.84 | 61.32 | 56.15 | 48.64 |
| Formulation 7 | 72.52 | 62.38 | 57.44 | 50.04 |
| Formulation 8 | 72.29 | 60.05 | 55.26 | 46.08 |
| Formulation 9 | 72.28 | 62.08 | 57.34 | 48.51 |
| Formulation 10 | 72.35 | 62.29 | 56.52 | 49.21 |
| Formulation 11 | 72.55 | 61.70 | 55.69 | 48.81 |
| Formulation 12 | 72.37 | 61.55 | 55.76 | 48.60 |
| Formulation 13 | 72.37 | 61.63 | 55.39 | 48.69 |
| Formulation 14 | 72.50 | 61.35 | 55.78 | 47.62 |
| Formulation 15 | 72.17 | 61.92 | 56.36 | 49.79 |
| Formulation 16 | 73.01 | 62.70 | 57.34 | 51.21 |
| Formulation 17 | 71.68 | 60.46 | 53.51 | 46.36 |
| Formulation 18 | 72.56 | 61.82 | 55.60 | 48.19 |
| Formulation 19 | 72.80 | 61.50 | 56.12 | 48.88 |
| Formulation 20 | 72.79 | 60.27 | 53.74 | 45.61 |
| Formulation 21 | 72.32 | 61.77 | 56.04 | 48.67 |
| Formulation 22 | 72.72 | 59.95 | 53.77 | 44.52 |

The results determined by the WCX analysis at a storage temperature of 40° C. are shown in Tables 68 to 70. During the 6-week storage period, the Acidic % showed no significant increase, and was measured to be +17.73% in average.

During the 6-week storage period, the Basic % showed no significant increase, and was measured to be +6.41% in average.

During the 6-week storage period, the Main % showed no significant decrease, and an average decrease of 24.14% was measured.

According to the results in Tables 65 to 70, it was confirmed that the formulations including an amino acid and optionally a surfactant had better stability under stress conditions than the formulations including a polyol and optionally a surfactant.

TABLE 71
SE-HPLC HMW (%) measurement results (photostability)

HMW (%)

| Formulation | Initial | Dark condition | Light exposure |
|---|---|---|---|
| Formulation 1 | 1.06 | 1.09 | 5.39 |
| Formulation 2 | 1.08 | 1.07 | 5.72 |
| Formulation 3 | 1.10 | 1.05 | 5.13 |
| Formulation 4 | 1.08 | 1.06 | 5.30 |
| Formulation 5 | 1.18 | 1.16 | 5.22 |
| Formulation 6 | 0.90 | 0.91 | 4.53 |
| Formulation 7 | 0.92 | 0.90 | 4.37 |
| Formulation 8 | 0.95 | 0.97 | 5.40 |
| Formulation 9 | 1.02 | 0.94 | 5.11 |
| Formulation 10 | 1.09 | 1.11 | 5.11 |
| Formulation 11 | 1.06 | 1.06 | 6.23 |
| Formulation 12 | 1.02 | 1.06 | 5.19 |
| Formulation 13 | 1.09 | 1.09 | 5.14 |
| Formulation 14 | 0.97 | 0.99 | 3.91 |
| Formulation 15 | 0.97 | 0.92 | 3.74 |
| Formulation 16 | 0.97 | 0.90 | 4.02 |
| Formulation 17 | 1.03 | 0.93 | 4.66 |
| Formulation 18 | 1.08 | 0.93 | 4.62 |
| Formulation 19 | 1.08 | 1.08 | 5.52 |
| Formulation 20 | 0.80 | 0.80 | 1.31 |
| Formulation 21 | 1.15 | 1.12 | 5.73 |
| Formulation 22 | 0.87 | 0.74 | 1.38 |

TABLE 72
SE-HPLC LMW (%) measurement results (photostability)

LMW (%)

| Formulation | Initial | Dark condition | Light exposure |
|---|---|---|---|
| Formulation 1 | 1.24 | 1.39 | 1.44 |
| Formulation 2 | 1.29 | 1.38 | 1.41 |
| Formulation 3 | 1.27 | 1.35 | 1.56 |
| Formulation 4 | 1.20 | 1.35 | 1.49 |
| Formulation 5 | 1.30 | 1.41 | 1.58 |
| Formulation 6 | 1.24 | 1.35 | 1.56 |
| Formulation 7 | 1.27 | 1.43 | 1.57 |
| Formulation 8 | 1.33 | 1.35 | 1.50 |
| Formulation 9 | 1.26 | 1.35 | 1.45 |
| Formulation 10 | 1.30 | 1.36 | 1.44 |
| Formulation 11 | 1.25 | 1.31 | 1.39 |
| Formulation 12 | 1.33 | 1.40 | 1.42 |
| Formulation 13 | 1.23 | 1.34 | 1.53 |
| Formulation 14 | 1.25 | 1.44 | 1.45 |
| Formulation 15 | 1.29 | 1.37 | 1.46 |
| Formulation 16 | 1.38 | 1.48 | 1.43 |
| Formulation 17 | 1.23 | 1.39 | 1.43 |
| Formulation 18 | 1.17 | 1.33 | 1.38 |
| Formulation 19 | 1.25 | 1.44 | 1.36 |
| Formulation 20 | 1.32 | 1.32 | 1.35 |
| Formulation 21 | 1.18 | 1.35 | 1.35 |
| Formulation 22 | 1.34 | 1.43 | 1.41 |

TABLE 73
SE-HPLC Monomer (%) measurement results (photostability)

Monomer (%)

| Formulation | Initial | Dark condition | Light exposure |
|---|---|---|---|
| Formulation 1 | 97.70 | 97.53 | 93.17 |
| Formulation 2 | 97.64 | 97.54 | 92.87 |
| Formulation 3 | 97.64 | 97.60 | 93.31 |
| Formulation 4 | 97.72 | 97.59 | 93.20 |
| Formulation 5 | 97.51 | 97.42 | 93.20 |
| Formulation 6 | 97.85 | 97.74 | 93.92 |
| Formulation 7 | 97.81 | 97.68 | 94.06 |
| Formulation 8 | 97.71 | 97.69 | 93.10 |
| Formulation 9 | 97.71 | 97.70 | 93.44 |
| Formulation 10 | 97.62 | 97.53 | 93.45 |
| Formulation 11 | 97.69 | 97.62 | 92.38 |
| Formulation 12 | 97.64 | 97.54 | 93.40 |
| Formulation 13 | 97.68 | 97.57 | 93.33 |
| Formulation 14 | 97.78 | 97.57 | 94.64 |
| Formulation 15 | 97.74 | 97.71 | 94.80 |
| Formulation 16 | 97.65 | 97.63 | 94.56 |
| Formulation 17 | 97.74 | 97.67 | 93.92 |
| Formulation 18 | 97.75 | 97.74 | 94.00 |
| Formulation 19 | 97.68 | 97.48 | 93.12 |
| Formulation 20 | 97.88 | 97.88 | 97.33 |
| Formulation 21 | 97.66 | 97.52 | 92.91 |
| Formulation 22 | 97.79 | 97.83 | 97.21 |

The results determined by the SE-HPLC analysis under conditions of an illuminance of not less than 1.2 million lux hours and an integrated near ultraviolet of not less than 200 watt hours/square meter are shown in Tables 71 to 73.

After light exposure, the HMW % increased by 0.5% to 5.2%, wherein an average increase of 4.27% was measured for the formulations including a surfactant and either a polyol or a salt (Formulations 1 to 5), an average increase of 3.24% was measured for the formulations including an amino acid and a surfactant (Formulations 6 to 9 and 20), an average increase of 4.05% was measured for the formulations including a polyol or a salt (Formulations 10 to 14), and an average increase of 2.80% was measured for the formulations including an amino acid (Formulations 15 to 18 and 22).

After light exposure, the LMW % showed no significant increase, and was measured to be +0.07% in average.

After light exposure, the Monomer % decreased by 0.6% to 5.2%, wherein an average decrease of 4.39% was measured for the formulations including a surfactant and either a polyol or a salt (Formulations 1 to 5), an average decrease of 3.37% was measured for the formulations including an amino acid and a surfactant (Formulations 6 to 9 and 20), an average decrease of 4.13% was measured for the formulations including a polyol or a salt (Formulations 10 to 14), and an average increase of 2.82% was measured for the formulations including an amino acid (Formulations 15 to 18 and 22).

TABLE 74
WCX Acidic (%) measurement results (photostability)

Acidic (%)

| Formulation | Initial | Dark condition | Light exposure |
|---|---|---|---|
| Formulation 1 | 20.08 | 20.83 | 28.09 |
| Formulation 2 | 19.69 | 20.81 | 28.27 |
| Formulation 3 | 21.16 | 20.83 | 28.89 |
| Formulation 4 | 20.16 | 20.66 | 28.51 |
| Formulation 5 | 19.81 | 20.51 | 27.28 |
| Formulation 6 | 19.85 | 20.31 | 26.81 |
| Formulation 7 | 19.81 | 20.67 | 27.84 |
| Formulation 8 | 20.58 | 20.96 | 30.90 |
| Formulation 9 | 20.29 | 20.91 | 28.63 |
| Formulation 10 | 20.30 | 20.71 | 27.68 |
| Formulation 11 | 20.17 | 20.74 | 29.04 |
| Formulation 12 | 20.27 | 20.72 | 28.56 |
| Formulation 13 | 20.14 | 20.59 | 28.17 |
| Formulation 14 | 19.99 | 20.20 | 23.74 |
| Formulation 15 | 20.35 | 20.46 | 25.84 |
| Formulation 16 | 19.55 | 20.30 | 27.10 |
| Formulation 17 | 21.31 | 20.84 | 28.06 |
| Formulation 18 | 19.92 | 20.85 | 26.12 |
| Formulation 19 | 19.80 | 20.65 | 27.19 |
| Formulation 20 | 20.04 | 20.58 | 23.60 |
| Formulation 21 | 20.16 | 20.69 | 27.97 |
| Formulation 22 | 20.03 | 20.87 | 23.27 |

TABLE 75
WCX Basic (%) measurement results (photostability)

Basic (%)

| Formulation | Initial | Dark condition | Light exposure |
|---|---|---|---|
| Formulation 1 | 7.50 | 7.69 | 21.63 |
| Formulation 2 | 7.58 | 7.25 | 22.12 |
| Formulation 3 | 7.40 | 7.37 | 22.26 |
| Formulation 4 | 7.43 | 7.62 | 22.66 |
| Formulation 5 | 7.34 | 7.80 | 22.56 |
| Formulation 6 | 7.31 | 7.69 | 23.72 |
| Formulation 7 | 7.67 | 7.61 | 22.90 |
| Formulation 8 | 7.13 | 7.10 | 22.13 |
| Formulation 9 | 7.42 | 7.68 | 21.86 |
| Formulation 10 | 7.35 | 7.55 | 20.91 |
| Formulation 11 | 7.28 | 7.42 | 22.53 |
| Formulation 12 | 7.36 | 7.17 | 21.15 |
| Formulation 13 | 7.48 | 7.35 | 20.20 |
| Formulation 14 | 7.51 | 7.69 | 22.21 |
| Formulation 15 | 7.48 | 7.71 | 19.46 |
| Formulation 16 | 7.44 | 7.67 | 20.89 |
| Formulation 17 | 7.01 | 7.45 | 19.96 |
| Formulation 18 | 7.52 | 7.19 | 20.53 |
| Formulation 19 | 7.40 | 7.55 | 21.54 |
| Formulation 20 | 7.17 | 7.29 | 10.93 |
| Formulation 21 | 7.52 | 7.47 | 21.06 |
| Formulation 22 | 7.25 | 7.15 | 10.41 |

TABLE 76
WCX Main (%) measurement results (photostability)

Main (%)

| Formulation | Initial | Dark condition | Light exposure |
|---|---|---|---|
| Formulation 1 | 72.42 | 71.48 | 50.28 |
| Formulation 2 | 72.72 | 71.93 | 49.61 |
| Formulation 3 | 71.44 | 71.80 | 48.85 |
| Formulation 4 | 72.41 | 71.72 | 48.83 |
| Formulation 5 | 72.85 | 71.69 | 50.16 |
| Formulation 6 | 72.84 | 72.00 | 49.46 |
| Formulation 7 | 72.52 | 71.71 | 49.26 |
| Formulation 8 | 72.29 | 71.94 | 46.96 |
| Formulation 9 | 72.28 | 71.41 | 49.51 |
| Formulation 10 | 72.35 | 71.74 | 51.41 |
| Formulation 11 | 72.55 | 71.84 | 48.43 |
| Formulation 12 | 72.37 | 72.11 | 50.29 |
| Formulation 13 | 72.37 | 72.06 | 51.63 |
| Formulation 14 | 72.50 | 72.11 | 54.04 |
| Formulation 15 | 72.17 | 71.83 | 54.70 |
| Formulation 16 | 73.01 | 72.03 | 52.01 |
| Formulation 17 | 71.68 | 71.71 | 51.98 |
| Formulation 18 | 72.56 | 71.96 | 53.35 |
| Formulation 19 | 72.80 | 71.81 | 51.26 |
| Formulation 20 | 72.79 | 72.13 | 65.47 |
| Formulation 21 | 72.32 | 71.84 | 50.97 |
| Formulation 22 | 72.72 | 71.97 | 66.32 |

The results determined by the WCX analysis under conditions of an illuminance of not less than 1.2 million lux hours and an integrated near ultraviolet of not less than 200 watt hours/square meter are shown in Tables 74 to 76.

After light exposure, the Acidic % increased by 2.4% to 9.9%, wherein an average increase of 7.48% was measured for the formulations including a surfactant and either a polyol or a salt (Formulations 1 to 5), an average increase of 6.87% was measured for the formulations including an amino acid and a surfactant (Formulations 6 to 9 and 20), an average increase of 6.85% was measured for the formulations including a polyol or a salt (Formulations 10 to 14), and an average increase of 5.41% was measured for the formulations including an amino acid (Formulations 15 to 18 and 22).

After light exposure, the Basic % increased by 3.3% to 16.0%, wherein an average increase of 14.70% was measured for the formulations including a surfactant and either a polyol or a salt (Formulations 1 to 5), an average increase of 12.83% was measured for the formulations including an amino acid and a surfactant (Formulations 6 to 9 and 20), an average increase of 13.96% was measured for the formulations including a polyol or a salt (Formulations 10 to 14), and an average increase of 10.82% was measured for the formulations including an amino acid (Formulations 15 to 18 and 22).

After light exposure, the Main % decreased by 5.7% to 25.0%, wherein an average decrease of 22.18% was measured for the formulations including a surfactant and either a polyol or a salt (Formulations 1 to 5), an average decrease of 19.71% was measured for the formulations including an amino acid and a surfactant (Formulations 6 to 9 and 20), an average decrease of 20.81% was measured for the formulations including a polyol or a salt (Formulations 10 to 14), and an average increase of 16.23% was measured for the formulations including an amino acid (Formulations 15 to 18 and 22).

TABLE 77
HIC Hydrophobic (%) measurement results (photostability)

Hydrophobic (%)

| Formulation | Initial | Dark condition | Light exposure |
|---|---|---|---|
| Formulation 1 | 5.25 | 6.29 | 19.11 |
| Formulation 2 | 5.39 | 6.26 | 19.74 |
| Formulation 3 | 5.31 | 6.26 | 20.86 |
| Formulation 4 | 5.45 | 6.33 | 21.05 |
| Formulation 5 | 5.76 | 6.27 | 21.02 |
| Formulation 6 | 5.57 | 6.61 | 23.40 |
| Formulation 7 | 5.90 | 6.76 | 22.93 |
| Formulation 8 | 5.62 | 6.57 | 23.54 |
| Formulation 9 | 5.68 | 6.62 | 20.73 |
| Formulation 10 | 5.77 | 6.42 | 18.06 |
| Formulation 11 | 5.65 | 6.49 | 21.83 |
| Formulation 12 | 5.78 | 6.54 | 19.07 |
| Formulation 13 | 5.73 | 6.58 | 17.67 |
| Formulation 14 | 5.89 | 6.54 | 18.04 |
| Formulation 15 | 5.95 | 6.90 | 16.58 |
| Formulation 16 | 6.03 | 6.98 | 18.73 |
| Formulation 17 | 5.89 | 6.66 | 16.97 |
| Formulation 18 | 5.95 | 6.71 | 16.04 |
| Formulation 19 | 6.08 | 6.55 | 17.91 |
| Formulation 20 | 5.86 | 6.61 | 12.50 |
| Formulation 21 | 6.00 | 6.63 | 18.09 |
| Formulation 22 | 6.10 | 6.96 | 11.70 |

TABLE 78
HIC Hydrophilic (%) measurement results (photostability)

Hydrophilic (%)

| Formulation | Initial | Dark condition | Light exposure |
|---|---|---|---|
| Formulation 1 | 3.93 | 3.73 | 6.04 |
| Formulation 2 | 3.98 | 3.70 | 6.21 |
| Formulation 3 | 3.89 | 3.66 | 5.90 |
| Formulation 4 | 3.88 | 3.71 | 5.94 |
| Formulation 5 | 3.69 | 3.42 | 5.52 |
| Formulation 6 | 3.79 | 3.60 | 5.29 |
| Formulation 7 | 3.75 | 3.66 | 5.39 |
| Formulation 8 | 3.76 | 3.58 | 5.95 |
| Formulation 9 | 3.79 | 3.58 | 5.87 |
| Formulation 10 | 3.83 | 3.61 | 5.89 |
| Formulation 11 | 3.77 | 3.63 | 6.45 |
| Formulation 12 | 3.77 | 3.63 | 5.87 |
| Formulation 13 | 3.75 | 3.58 | 5.87 |
| Formulation 14 | 3.73 | 3.60 | 5.12 |
| Formulation 15 | 3.63 | 3.60 | 5.01 |
| Formulation 16 | 3.61 | 3.54 | 5.12 |
| Formulation 17 | 3.63 | 3.54 | 5.54 |
| Formulation 18 | 3.64 | 3.55 | 5.56 |
| Formulation 19 | 3.74 | 3.63 | 6.03 |
| Formulation 20 | 3.45 | 3.37 | 3.56 |
| Formulation 21 | 3.61 | 3.62 | 6.09 |
| Formulation 22 | 3.49 | 3.48 | 3.59 |

TABLE 79
HIC Main (%) measurement results (photostability)

Main (%)

| Formulation | Initial | Dark condition | Light exposure |
|---|---|---|---|
| Formulation 1 | 90.81 | 89.98 | 74.84 |
| Formulation 2 | 90.62 | 90.05 | 74.05 |
| Formulation 3 | 90.79 | 90.08 | 73.24 |
| Formulation 4 | 90.67 | 89.96 | 73.01 |
| Formulation 5 | 90.56 | 90.30 | 73.46 |
| Formulation 6 | 90.64 | 89.79 | 71.31 |
| Formulation 7 | 90.35 | 89.58 | 71.68 |
| Formulation 8 | 90.62 | 89.85 | 70.51 |
| Formulation 9 | 90.54 | 89.80 | 73.40 |
| Formulation 10 | 90.40 | 89.97 | 76.05 |
| Formulation 11 | 90.58 | 89.89 | 71.72 |
| Formulation 12 | 90.45 | 89.84 | 75.06 |
| Formulation 13 | 90.52 | 89.83 | 76.46 |
| Formulation 14 | 90.38 | 89.86 | 76.85 |
| Formulation 15 | 90.42 | 89.50 | 78.41 |
| Formulation 16 | 90.37 | 89.48 | 76.14 |
| Formulation 17 | 90.48 | 89.80 | 77.49 |
| Formulation 18 | 90.41 | 89.74 | 78.40 |
| Formulation 19 | 90.19 | 89.82 | 76.06 |
| Formulation 20 | 90.69 | 90.02 | 83.94 |
| Formulation 21 | 90.39 | 89.76 | 75.82 |
| Formulation 22 | 90.40 | 89.56 | 84.71 |

The results determined by the HIC analysis under conditions of an illuminance of not less than 1.2 million lux hours and an integrated near ultraviolet of not less than 200 watt hours/square meter are shown in Tables 77 to 79.

After light exposure, the Hydrophobic % increased by 5% to 17%. The smallest increase was measured to be +9.68%, +9.33%, +5.89%, and +4.74% for Formulation 15, Formulation 18, Formulation 20, and Formulation 22, respectively, and the increase for the rest of the formulations except for the formulations with the smallest increases was measured to be +12.70% in average.

After light exposure, the Hydrophilic % increased by 0.1% to 3.0%, wherein an average increase of 2.28% was measured for the formulations including a surfactant and either a polyol or a salt (Formulations 1 to 5), an average increase of 1.65% was measured for the formulations including an amino acid and a surfactant (Formulations 6 to 9 and 20), an average increase of 2.23% was measured for the formulations including a polyol or a salt (Formulations 10 to 14), and an average increase of 1.42% was measured for the formulations including an amino acid (Formulations 15 to 18 and 22).

After light exposure, the Main % decreased by 5% to 19%. The smallest decrease was measured to be −11.09%, −11.34%, −6.08%, and −4.85% for Formulation 15, Formulation 18, Formulation 20, and Formulation 22, respectively, and the decrease for the rest of the formulations except for the formulations with the smallest decrease was measured to be −14.76% in average.

According to the results in Tables 71 to 79, the formulations including an amino acid and optionally a surfactant were confirmed to have better photostability under stress conditions than the formulations including a polyol and optionally a surfactant.

TABLE 80
SE-HPLC HMW (%) measurement results (freeze-thaw stability)

HMW (%)

| Formulation | Initial | 5 freeze-thaw cycles |
|---|---|---|
| Formulation 1 | 1.06 | 1.04 |
| Formulation 2 | 1.08 | 1.10 |
| Formulation 3 | 1.10 | 1.03 |
| Formulation 4 | 1.08 | 1.08 |
| Formulation 5 | 1.18 | 1.22 |
| Formulation 6 | 0.90 | 0.95 |
| Formulation 7 | 0.92 | 0.94 |
| Formulation 8 | 0.95 | 0.96 |
| Formulation 9 | 1.02 | 0.95 |
| Formulation 10 | 1.09 | 1.16 |
| Formulation 11 | 1.06 | 1.11 |
| Formulation 12 | 1.02 | 1.05 |
| Formulation 13 | 1.09 | 1.07 |
| Formulation 14 | 0.97 | 1.10 |
| Formulation 15 | 0.97 | 1.00 |
| Formulation 16 | 0.97 | 1.04 |
| Formulation 17 | 1.03 | 1.04 |
| Formulation 18 | 1.08 | 1.00 |
| Formulation 19 | 1.08 | 1.32 |
| Formulation 20 | 0.80 | 0.83 |
| Formulation 21 | 1.15 | 1.31 |
| Formulation 22 | 0.87 | 0.88 |

TABLE 81
SE-HPLC LMW (%) measurement results (freeze-thaw stability)

LMW (%)

| Formulation | Initial | 5 freeze-thaw cycles |
|---|---|---|
| Formulation 1 | 1.24 | 1.58 |
| Formulation 2 | 1.29 | 1.64 |
| Formulation 3 | 1.27 | 1.58 |
| Formulation 4 | 1.20 | 1.60 |
| Formulation 5 | 1.30 | 1.51 |
| Formulation 6 | 1.24 | 1.60 |
| Formulation 7 | 1.27 | 1.60 |
| Formulation 8 | 1.33 | 1.58 |
| Formulation 9 | 1.26 | 1.67 |
| Formulation 10 | 1.30 | 1.61 |
| Formulation 11 | 1.25 | 1.57 |
| Formulation 12 | 1.33 | 1.52 |
| Formulation 13 | 1.23 | 1.56 |
| Formulation 14 | 1.25 | 1.49 |
| Formulation 15 | 1.29 | 1.51 |
| Formulation 16 | 1.38 | 1.78 |
| Formulation 17 | 1.23 | 1.61 |
| Formulation 18 | 1.17 | 1.62 |
| Formulation 19 | 1.25 | 1.66 |
| Formulation 20 | 1.32 | 1.54 |
| Formulation 21 | 1.18 | 1.49 |
| Formulation 22 | 1.34 | 1.60 |

TABLE 82
SE-HPLC Monomer (%) measurement
results (freeze-thaw stability)

Monomer (%)

| Formulation | Initial | 5 freeze-thaw cycles |
|---|---|---|
| Formulation 1 | 97.70 | 97.38 |
| Formulation 2 | 97.64 | 97.26 |
| Formulation 3 | 97.64 | 97.39 |
| Formulation 4 | 97.72 | 97.31 |
| Formulation 5 | 97.51 | 97.27 |
| Formulation 6 | 97.85 | 97.44 |
| Formulation 7 | 97.81 | 97.46 |
| Formulation 8 | 97.71 | 97.45 |
| Formulation 9 | 97.71 | 97.37 |
| Formulation 10 | 97.62 | 97.23 |
| Formulation 11 | 97.69 | 97.33 |
| Formulation 12 | 97.64 | 97.44 |
| Formulation 13 | 97.68 | 97.38 |
| Formulation 14 | 97.78 | 97.41 |
| Formulation 15 | 97.74 | 97.49 |
| Formulation 16 | 97.65 | 97.18 |
| Formulation 17 | 97.74 | 97.35 |
| Formulation 18 | 97.75 | 97.38 |
| Formulation 19 | 97.68 | 97.02 |
| Formulation 20 | 97.88 | 97.64 |
| Formulation 21 | 97.66 | 97.21 |
| Formulation 22 | 97.79 | 97.53 |

The results determined by the SE-HPLC analysis after repeating a freeze-thaw cycle 5 times under conditions of room temperature and a temperature of −60° C. or lower are shown in Tables 80 to 82.

After 5 freeze-thaw cycles, the HMW % showed no significant increase, and was measured to be +0.03% in average.

After 5 freeze-thaw cycles, the LMW % showed no significant increase, and was measured to be +0.32% in average.

After 5 freeze-thaw cycles, the Monomer % showed no significant decrease, and was measured to be −0.35% in average.

TABLE 83
WCX Acidic (%) measurement results (freeze-thaw stability)

Acidic (%)

| Formulation | Initial | 5 freeze-thaw cycles |
|---|---|---|
| Formulation 1 | 20.08 | 19.39 |
| Formulation 2 | 19.69 | 19.60 |
| Formulation 3 | 21.16 | 19.28 |
| Formulation 4 | 20.16 | 18.92 |
| Formulation 5 | 19.81 | 18.75 |
| Formulation 6 | 19.85 | 19.03 |
| Formulation 7 | 19.81 | 19.19 |
| Formulation 8 | 20.58 | 19.62 |
| Formulation 9 | 20.29 | 19.55 |
| Formulation 10 | 20.30 | 19.61 |
| Formulation 11 | 20.17 | 18.89 |
| Formulation 12 | 20.27 | 18.95 |
| Formulation 13 | 20.14 | 19.41 |
| Formulation 14 | 19.99 | 18.84 |
| Formulation 15 | 20.35 | 19.07 |
| Formulation 16 | 19.55 | 19.34 |
| Formulation 17 | 21.31 | 18.87 |
| Formulation 18 | 19.92 | 19.41 |
| Formulation 19 | 19.80 | 19.12 |
| Formulation 20 | 20.04 | 18.92 |
| Formulation 21 | 20.16 | 19.30 |
| Formulation 22 | 20.03 | 19.17 |

TABLE 84
WCX Basic (%) measurement results (freeze-thaw stability)

Basic (%)

| Formulation | Initial | 5 freeze-thaw cycles |
|---|---|---|
| Formulation 1 | 7.50 | 8.09 |
| Formulation 2 | 7.58 | 7.92 |
| Formulation 3 | 7.40 | 8.35 |
| Formulation 4 | 7.43 | 8.47 |
| Formulation 5 | 7.34 | 8.81 |
| Formulation 6 | 7.31 | 8.29 |
| Formulation 7 | 7.67 | 8.46 |
| Formulation 8 | 7.13 | 8.20 |
| Formulation 9 | 7.42 | 8.28 |
| Formulation 10 | 7.35 | 8.24 |
| Formulation 11 | 7.28 | 8.38 |
| Formulation 12 | 7.36 | 8.49 |
| Formulation 13 | 7.48 | 8.12 |
| Formulation 14 | 7.51 | 8.63 |
| Formulation 15 | 7.48 | 8.37 |
| Formulation 16 | 7.44 | 8.18 |
| Formulation 17 | 7.01 | 8.76 |
| Formulation 18 | 7.52 | 8.48 |
| Formulation 19 | 7.40 | 8.45 |
| Formulation 20 | 7.17 | 8.52 |
| Formulation 21 | 7.52 | 8.23 |
| Formulation 22 | 7.25 | 8.35 |

TABLE 85
WCX Main (%) measurement results (freeze-thaw stability)

Main (%)

| Formulation | Initial | 5 freeze-thaw cycles |
|---|---|---|
| Formulation 1 | 72.42 | 72.51 |
| Formulation 2 | 72.72 | 72.48 |
| Formulation 3 | 71.44 | 72.36 |
| Formulation 4 | 72.41 | 72.61 |
| Formulation 5 | 72.85 | 72.44 |
| Formulation 6 | 72.84 | 72.68 |
| Formulation 7 | 72.52 | 72.35 |
| Formulation 8 | 72.29 | 72.18 |
| Formulation 9 | 72.28 | 72.17 |
| Formulation 10 | 72.35 | 72.15 |
| Formulation 11 | 72.55 | 72.73 |
| Formulation 12 | 72.37 | 72.56 |
| Formulation 13 | 72.37 | 72.47 |
| Formulation 14 | 72.50 | 72.52 |
| Formulation 15 | 72.17 | 72.55 |
| Formulation 16 | 73.01 | 72.49 |
| Formulation 17 | 71.68 | 72.37 |
| Formulation 18 | 72.56 | 72.11 |
| Formulation 19 | 72.80 | 72.43 |
| Formulation 20 | 72.79 | 72.56 |
| Formulation 21 | 72.32 | 72.46 |
| Formulation 22 | 72.72 | 72.48 |

The results determined by the WCX analysis after repeating a freeze-thaw cycle 5 times under conditions of room temperature and a temperature of −60° C. or lower are shown in Tables 83 to 85.

After 5 freeze-thaw cycles, the Acidic % showed no significant increase, and was measured to be −0.96% in average.

After 5 freeze-thaw cycles, the Basic % showed no significant increase, and was measured to be +0.98% in average.

After 5 freeze-thaw cycles, the Main % showed no significant decrease, and was measured to be −0.01% in average.

According to the results in Tables 80 to 85, all formulations were confirmed to be stable under the freeze-thaw stress conditions.

TABLE 86
SE-HPLC HMW (%) measurement results (agitation stability)

HMW (%)

| Formulation | 0 rpm | 300 rpm |
|---|---|---|
| Formulation 1 | 0.98 | 1.10 |
| Formulation 2 | 1.01 | 1.07 |
| Formulation 3 | 0.93 | 1.02 |
| Formulation 4 | 0.99 | 1.08 |
| Formulation 5 | 1.11 | 1.14 |
| Formulation 6 | 0.82 | 0.87 |
| Formulation 7 | 0.82 | 0.89 |
| Formulation 8 | 0.88 | 0.90 |
| Formulation 9 | 0.89 | 1.02 |
| Formulation 10 | 0.97 | 1.09 |
| Formulation 11 | 0.97 | 1.10 |
| Formulation 12 | 1.02 | 0.92 |
| Formulation 13 | 0.98 | 1.09 |
| Formulation 14 | 1.05 | 0.94 |
| Formulation 15 | 0.88 | 0.93 |
| Formulation 16 | 0.84 | 0.85 |
| Formulation 17 | 0.93 | 0.91 |
| Formulation 18 | 0.92 | 1.02 |
| Formulation 19 | 1.04 | 1.06 |
| Formulation 20 | 0.71 | 0.81 |
| Formulation 21 | 1.08 | 1.11 |
| Formulation 22 | 0.71 | 0.78 |

TABLE 87
SE-HPLC LMW (%) measurement results (agitation stability)

LMW (%)

| Formulation | 0 rpm | 300 rpm |
|---|---|---|
| Formulation 1 | 1.51 | 1.60 |
| Formulation 2 | 1.50 | 1.49 |
| Formulation 3 | 1.56 | 1.45 |
| Formulation 4 | 1.43 | 1.43 |
| Formulation 5 | 1.48 | 1.36 |
| Formulation 6 | 1.48 | 1.57 |
| Formulation 7 | 1.60 | 1.41 |
| Formulation 8 | 1.52 | 1.39 |
| Formulation 9 | 1.52 | 1.47 |
| Formulation 10 | 1.49 | 1.49 |
| Formulation 11 | 1.46 | 1.43 |
| Formulation 12 | 1.49 | 1.31 |
| Formulation 13 | 1.47 | 1.40 |
| Formulation 14 | 1.63 | 1.44 |
| Formulation 15 | 1.53 | 1.52 |
| Formulation 16 | 1.56 | 1.46 |
| Formulation 17 | 1.55 | 1.61 |
| Formulation 18 | 1.49 | 1.58 |
| Formulation 19 | 1.51 | 1.42 |
| Formulation 20 | 1.51 | 1.27 |
| Formulation 21 | 1.56 | 1.48 |
| Formulation 22 | 1.50 | 1.46 |

TABLE 88
SE-HPLC Monomer (%) measurement results (agitation stability)

Monomer (%)

| Formulation | 0 rpm | 300 rpm |
|---|---|---|
| Formulation 1 | 97.51 | 97.30 |
| Formulation 2 | 97.49 | 97.43 |
| Formulation 3 | 97.52 | 97.53 |
| Formulation 4 | 97.59 | 97.50 |
| Formulation 5 | 97.42 | 97.50 |
| Formulation 6 | 97.70 | 97.55 |
| Formulation 7 | 97.57 | 97.70 |
| Formulation 8 | 97.61 | 97.72 |
| Formulation 9 | 97.59 | 97.51 |
| Formulation 10 | 97.55 | 97.41 |
| Formulation 11 | 97.57 | 97.47 |
| Formulation 12 | 97.49 | 97.77 |
| Formulation 13 | 97.56 | 97.51 |
| Formulation 14 | 97.33 | 97.62 |
| Formulation 15 | 97.58 | 97.55 |
| Formulation 16 | 97.60 | 97.68 |
| Formulation 17 | 97.52 | 97.47 |
| Formulation 18 | 97.59 | 97.40 |
| Formulation 19 | 97.44 | 97.52 |
| Formulation 20 | 97.78 | 97.92 |
| Formulation 21 | 97.36 | 97.40 |
| Formulation 22 | 97.79 | 97.75 |

The results determined by the SE-HPLC analysis after stirring under conditions of 300 rpm are shown in Tables 86 to 88. After stirring, the HMW % showed no significant increase, and was measured to be +0.05% in average. After stirring, the LMW % showed no significant increase, and was measured to be −0.06% in average. After stirring, the Monomer % showed no significant decrease, and was measured to be 0.00% in average.

TABLE 89
WCX Acidic (%) measurement results (agitation stability)

Acidic (%)

| Formulation | 0 rpm | 300 rpm |
|---|---|---|
| Formulation 1 | 20.64 | 20.71 |
| Formulation 2 | 20.62 | 20.36 |
| Formulation 3 | 20.44 | 20.53 |
| Formulation 4 | 20.59 | 20.26 |
| Formulation 5 | 20.39 | 19.86 |
| Formulation 6 | 19.69 | 20.07 |
| Formulation 7 | 20.29 | 19.93 |
| Formulation 8 | 21.07 | 20.77 |
| Formulation 9 | 20.70 | 20.21 |
| Formulation 10 | 20.29 | 20.24 |
| Formulation 11 | 20.64 | 20.18 |
| Formulation 12 | 20.82 | 20.16 |
| Formulation 13 | 20.58 | 20.32 |
| Formulation 14 | 19.71 | 19.73 |
| Formulation 15 | 20.10 | 20.25 |
| Formulation 16 | 20.33 | 20.26 |
| Formulation 17 | 21.06 | 20.66 |
| Formulation 18 | 20.65 | 20.11 |
| Formulation 19 | 20.52 | 20.57 |
| Formulation 20 | 20.03 | 19.99 |
| Formulation 21 | 20.80 | 20.44 |
| Formulation 22 | 20.35 | 19.96 |

TABLE 90
WCX Basic (%) measurement results (agitation stability)

Basic (%)

| Formulation | 0 rpm | 300 rpm |
|---|---|---|
| Formulation 1 | 8.13 | 7.59 |
| Formulation 2 | 7.59 | 7.64 |
| Formulation 3 | 7.53 | 7.97 |
| Formulation 4 | 7.51 | 7.98 |
| Formulation 5 | 7.92 | 7.55 |
| Formulation 6 | 7.74 | 8.03 |
| Formulation 7 | 7.70 | 7.55 |
| Formulation 8 | 7.66 | 7.67 |
| Formulation 9 | 7.58 | 7.89 |
| Formulation 10 | 7.76 | 7.63 |
| Formulation 11 | 7.63 | 7.60 |
| Formulation 12 | 7.84 | 7.61 |
| Formulation 13 | 7.34 | 7.59 |
| Formulation 14 | 7.92 | 8.30 |
| Formulation 15 | 7.64 | 7.74 |
| Formulation 16 | 7.43 | 8.02 |
| Formulation 17 | 7.15 | 7.57 |
| Formulation 18 | 7.71 | 7.69 |
| Formulation 19 | 7.38 | 7.78 |
| Formulation 20 | 7.28 | 7.45 |
| Formulation 21 | 7.36 | 7.52 |
| Formulation 22 | 7.18 | 7.51 |

TABLE 91
WCX Main (%) measurement results (agitation stability)

Main (%)

| Formulation | 0 rpm | 300 rpm |
|---|---|---|
| Formulation 1 | 71.23 | 71.70 |
| Formulation 2 | 71.79 | 72.01 |
| Formulation 3 | 72.03 | 71.49 |
| Formulation 4 | 71.89 | 71.76 |
| Formulation 5 | 71.68 | 72.59 |
| Formulation 6 | 72.57 | 71.89 |
| Formulation 7 | 72.01 | 72.52 |
| Formulation 8 | 71.27 | 71.56 |
| Formulation 9 | 71.73 | 71.90 |
| Formulation 10 | 71.95 | 72.13 |
| Formulation 11 | 71.73 | 72.22 |
| Formulation 12 | 71.34 | 72.23 |
| Formulation 13 | 72.08 | 72.09 |
| Formulation 14 | 72.37 | 71.97 |
| Formulation 15 | 72.26 | 72.01 |
| Formulation 16 | 72.25 | 71.71 |
| Formulation 17 | 71.79 | 71.78 |
| Formulation 18 | 71.64 | 72.20 |
| Formulation 19 | 72.10 | 71.65 |
| Formulation 20 | 72.69 | 72.56 |
| Formulation 21 | 71.84 | 72.04 |
| Formulation 22 | 72.47 | 72.53 |

The results determined by the WCX analysis after stirring under conditions of 300 rpm are shown in Tables 89 to 91. After stirring, the Acidic % showed no significant increase, and was measured to be −0.22% in average. After stirring, the Basic % showed no significant increase, and was measured to be +0.13% in average. After stirring, the Main % showed no significant increase, and was measured to be +0.08% in average. According to the results in Tables 86 to 91, all formulations were confirmed to be stable under the stirring stress conditions.

Discussion of results: The formulations including an amino acid stabilizer were shown to be stable under various stress conditions (thermal, light, and stirring stress) with or without a surfactant. Meanwhile, the formulations including a polyol stabilizer tended to have less stability compared to those including an amino acid stabilizer.

### Test Example 4: Stability Analysis of Formulation Including Only Stabilizer

Aqueous risankizumab liquid formulations of compositions shown in Table 92 below were prepared, and then analyzed for thermal stability.

TABLE 92
Formulation compositions including only stabilizer

| | Protein | | | | |
|---|---|---|---|---|---|
| | concentration of | Buffer | | | |
| Formulation | risankizumab | solution | pH | Stabilizer | Surfactant |
| Formulation 1 | 150 mg/mL | N/A | 5.7 | 4% sorbitol | N/A |
| Formulation 2 | | | | 4% mannitol | |
| Formulation 3 | | | | 7% sucrose | |
| Formulation 4 | | | | 7% trehalose | |
| Formulation 5 | | | | 2.3% lysine | |
| Formulation 6 | | | | 2.6% arginine | |
| Formulation 7 | | | | 2.5% proline | |
| Formulation 8 | | | | 150 mM histidine | |

Preparation of samples: Formulation samples including only stabilizers without buffer solutions and surfactants were collected after storage under thermal temperature stress conditions to determine stability thereof. Dialysis was performed by using each prepared buffer solution, and formulation samples were prepared in a desired protein concentration. The prepared formulation was sterile filtered, filled with 0.3 mL in a tube, and exposed to temperature stress conditions. For the thermal stability, the samples were stored and collected at Weeks 1, 2, and 4 under temperature conditions of 40±2° C.

Results: Results of measuring the thermal stability are shown in Tables 93 to 95 below.

TABLE 93
SE-HPLC HMW (%) measurement results (thermal stability)

HMW (%)

| Formulation | Initial | Week 1 | Week 2 | Week 4 |
|---|---|---|---|---|
| Formulation 1 | 1.59 | 2.27 | 2.73 | 3.27 |
| Formulation 2 | 1.74 | 2.20 | 2.93 | 3.39 |
| Formulation 3 | 1.44 | 1.80 | 2.55 | 3.06 |
| Formulation 4 | 1.68 | 2.20 | 2.97 | 3.41 |
| Formulation 5 | 1.19 | 1.50 | 1.95 | 2.34 |
| Formulation 6 | 1.24 | 1.51 | 1.82 | 2.17 |
| Formulation 7 | 1.35 | 1.82 | 2.28 | 2.65 |
| Formulation 8 | 1.95 | 2.16 | 2.63 | 2.95 |

TABLE 94
SE-HPLC LMW (%) measurement results (thermal stability)

LMW (%)

| Formulation | Initial | Week 1 | Week 2 | Week 4 |
|---|---|---|---|---|
| Formulation 1 | 0.76 | 1.61 | 1.24 | 2.06 |
| Formulation 2 | 0.74 | 1.44 | 1.17 | 1.97 |
| Formulation 3 | 0.88 | 1.53 | 1.33 | 2.15 |
| Formulation 4 | 0.79 | 1.54 | 1.29 | 2.12 |
| Formulation 5 | 0.82 | 1.61 | 1.33 | 2.23 |
| Formulation 6 | 0.88 | 1.79 | 1.36 | 2.27 |
| Formulation 7 | 0.67 | 1.68 | 1.21 | 2.07 |
| Formulation 8 | 0.60 | 1.49 | 1.47 | 2.38 |

TABLE 95
SE-HPLC Monomer (%) measurement results (thermal stability)

Monomer (%)

| Formulation | Initial | Week 1 | Week 2 | Week 4 |
|---|---|---|---|---|
| Formulation 1 | 97.64 | 96.12 | 96.02 | 94.67 |
| Formulation 2 | 97.52 | 96.36 | 95.90 | 94.64 |
| Formulation 3 | 97.68 | 96.66 | 96.12 | 94.79 |
| Formulation 4 | 97.53 | 96.25 | 95.74 | 94.47 |
| Formulation 5 | 97.99 | 96.90 | 96.71 | 95.42 |
| Formulation 6 | 97.88 | 96.70 | 96.82 | 95.56 |
| Formulation 7 | 97.98 | 96.49 | 96.51 | 95.28 |
| Formulation 8 | 97.44 | 96.35 | 95.91 | 94.67 |

The results determined by the SE-HPLC analysis at a storage temperature of 40° C. are shown in Tables 93 to 95.

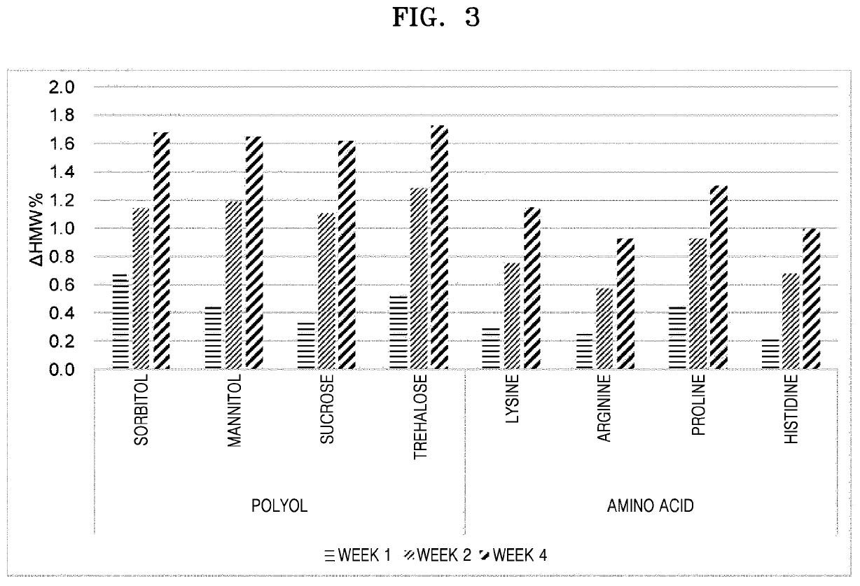

During the 4-week storage period, the HMW % increased by 0.9% to 1.7%, wherein an average increase of 1.67% was measured for the formulations including only a polyol (Formulations 1 to 4), and an average increase of 1.10% was measured for the formulations including only an amino acid (Formulations 5 to 8). Changes in the HMW % (ΔHMW %) at Weeks 1, 2, and 4 relative to the initial period are shown in FIG. 3.

During the 4-week storage period, the LMW % showed no significant increase, and was measured to be +1.39% in average.

During the 4-week storage period, the Monomer % decreased by 2.3% to 3.1% as the HMW % increased, wherein an average decrease of 2.95% was measured for the formulations including only a polyol (Formulations 1 to 4), and an average decrease of 2.59% was measured for the formulations including only an amino acid (Formulations 5 to 8).

According to the results in Tables 92 to 95, the formulations including an amino acid were confirmed to have better thermal temperature stability than the formulations including a polyol.

Discussion of results: According to the results in Tables 93 to 95, the formulations including only an amino acid stabilizer were shown to be more stable under the thermal stress conditions than formulations including only a polyol stabilizer.

### Test Example 5: Concentration-Dependent Stability Analysis of Formulation Including Proline Stabilizer

Aqueous risankizumab liquid formulations of compositions shown in Table 96 below were prepared, and then analyzed for thermal stability, photostability, freeze-thaw stability, and agitation stability.

TABLE 96
Formulation compositions with different stabilizer concentrations

| | Protein | | | | |
|---|---|---|---|---|---|
| | concentration | | | | |
| | of | | | | |
| | risankizumab | Histidine | | Proline | Polysorbate |
| Formulation | (mg/mL) | (mM) | PH | (%) | 20 (%) |
| Formulation 1 | 170 | 10 | 5.0 | 2.0 | 0.02 |
| Formulation 2 | 130 | 10 | 5.0 | 2.0 | 0.03 |
| Formulation 3 | 150 | 10 | 5.0 | 2.5 | 0.01 |
| Formulation 4 | 130 | 10 | 5.0 | 3.0 | 0.01 |
| Formulation 5 | 170 | 10 | 5.0 | 3.0 | 0.03 |
| Formulation 6 | 130 | 35 | 5.0 | 2.0 | 0.01 |
| Formulation 7 | 150 | 35 | 5.0 | 3.0 | 0.03 |
| Formulation 8 | 170 | 60 | 5.0 | 2.0 | 0.03 |
| Formulation 9 | 130 | 60 | 5.0 | 2.5 | 0.02 |
| Formulation 10 | 170 | 60 | 5.0 | 3.0 | 0.01 |
| Formulation 11 | 170 | 35 | 6.0 | 2.5 | 0.01 |
| Formulation 12 | 150 | 35 | 6.0 | 2.5 | 0.02 |
| Formulation 13 | 150 | 35 | 6.0 | 2.5 | 0.02 |
| Formulation 14 | 150 | 35 | 6.0 | 2.5 | 0.02 |
| Formulation 15 | 150 | 60 | 6.0 | 2.0 | 0.01 |
| Formulation 16 | 130 | 60 | 6.0 | 3.0 | 0.03 |
| Formulation 17 | 130 | 10 | 6.7 | 2.0 | 0.01 |
| Formulation 18 | 170 | 10 | 6.7 | 2.0 | 0.03 |
| Formulation 19 | 170 | 10 | 6.7 | 3.0 | 0.01 |
| Formulation 20 | 130 | 10 | 6.7 | 3.0 | 0.03 |
| Formulation 21 | 170 | 60 | 6.7 | 2.0 | 0.01 |
| Formulation 22 | 130 | 60 | 6.7 | 2.0 | 0.03 |
| Formulation 23 | 130 | 60 | 6.7 | 3.0 | 0.01 |
| Formulation 24 | 170 | 60 | 6.7 | 3.0 | 0.03 |

Preparation of samples: In order to determine an appropriate concentration range for stability, formulation samples were prepared by designing a total of 5 factors (protein concentration, pH, concentration of histidine buffer, concentration of proline stabilizer, and concentration of surfactant) based on Design of Experiments (DoE), and then collected after storage. Dialysis was performed by using each prepared buffer solution, and formulation samples were prepared in a desired protein concentration. The prepared formulation was sterile filtered, filled with 1 mL in a syringe, and exposed to various stress conditions. The stress conditions are as follows. For the thermal stability, the samples were stored and collected at Weeks 4 and 6 under temperature conditions of 40±2° C. For the photostability, the samples were stored and collected under conditions of an illuminance of not less than 1.2 million lux hours and an integrated near ultraviolet of not less than 200 watt hours/square meter. For the freeze-thaw stability, the samples were collected after repeating a freeze-thaw cycle five times under conditions of room temperature and a temperature of −60° C. or lower. For the agitation stability, the samples were collected after stirring under conditions of 0 rpm and 400 rpm.

Analysis method: A prediction model was established by considering main effects, interaction effects, and secondary effects for a total of 5 factors (protein concentration, pH, histidine concentration, proline concentration, and surfactant concentration), and then the thermal stability, photostability, freeze-thaw stability, and agitation stability at various concentrations were determined.

Results: Results of measuring the thermal stability, photostability, freeze-thaw stability, and agitation stability are shown in Tables 97 to 123 below.

TABLE 97
SE-HPLC HMW (%) measurement results (thermal stability)

| | HMW (%) | | |
|---|---|---|---|
| Formulation | Initial | Week 4 | Week 6 |
| Formulation 1 | 1.12 | 2.68 | 3.46 |
| Formulation 2 | 1.13 | 2.03 | 2.48 |
| Formulation 3 | 1.06 | 2.14 | 2.73 |
| Formulation 4 | 0.89 | 1.84 | 2.39 |
| Formulation 5 | 1.03 | 2.23 | 2.89 |
| Formulation 6 | 0.99 | 2.33 | 3.07 |
| Formulation 7 | 1.02 | 2.53 | 3.40 |
| Formulation 8 | 0.97 | 3.38 | 4.24 |
| Formulation 9 | 0.71 | 2.48 | 3.31 |
| Formulation 10 | 1.00 | 2.82 | 3.70 |
| Formulation 11 | 1.29 | 2.01 | 2.44 |
| Formulation 12 | 1.25 | 1.91 | 2.30 |
| Formulation 13 | 1.27 | 1.93 | 2.33 |
| Formulation 14 | 1.35 | 1.86 | 2.24 |
| Formulation 15 | 1.23 | 1.74 | 2.15 |
| Formulation 16 | 0.86 | 1.46 | 1.79 |
| Formulation 17 | 1.32 | 2.71 | 3.11 |
| Formulation 18 | 1.50 | 3.38 | 3.97 |
| Formulation 19 | 1.50 | 3.38 | 4.06 |
| Formulation 20 | 1.27 | 2.49 | 2.95 |
| Formulation 21 | 1.13 | 2.26 | 2.59 |
| Formulation 22 | 1.06 | 1.77 | 2.21 |
| Formulation 23 | 1.08 | 1.74 | 2.07 |
| Formulation 24 | 1.20 | 2.20 | 2.53 |

TABLE 98
SE-HPLC LMW (%) measurement results (thermal stability)

| | LMW (%) | | |
|---|---|---|---|
| Formulation | Initial | Week 4 | Week 6 |
| Formulation 1 | 0.39 | 3.40 | 4.78 |
| Formulation 2 | 0.44 | 3.43 | 4.65 |
| Formulation 3 | 0.42 | 3.39 | 4.73 |
| Formulation 4 | 0.39 | 3.48 | 4.85 |
| Formulation 5 | 0.38 | 3.38 | 4.69 |
| Formulation 6 | 0.39 | 4.62 | 6.36 |
| Formulation 7 | 0.39 | 4.59 | 6.41 |
| Formulation 8 | 0.40 | 5.09 | 6.88 |
| Formulation 9 | 0.31 | 5.07 | 6.90 |
| Formulation 10 | 0.41 | 4.83 | 6.62 |
| Formulation 11 | 0.37 | 2.17 | 3.15 |
| Formulation 12 | 0.37 | 2.23 | 3.18 |
| Formulation 13 | 0.36 | 2.14 | 3.20 |
| Formulation 14 | 0.45 | 2.22 | 3.21 |
| Formulation 15 | 0.35 | 2.21 | 3.26 |
| Formulation 16 | 0.37 | 2.23 | 3.22 |
| Formulation 17 | 0.28 | 2.52 | 3.72 |
| Formulation 18 | 0.35 | 2.52 | 3.82 |
| Formulation 19 | 0.31 | 2.64 | 4.05 |
| Formulation 20 | 0.33 | 2.57 | 3.83 |
| Formulation 21 | 0.27 | 2.85 | 4.16 |
| Formulation 22 | 0.33 | 2.85 | 4.21 |
| Formulation 23 | 0.30 | 2.89 | 4.36 |
| Formulation 24 | 0.34 | 2.86 | 4.36 |

TABLE 99
SE-HPLC Monomer (%) measurement results (thermal stability)

| | Monomer (%) | | |
|---|---|---|---|
| Formulation | Initial | Week 4 | Week 6 |
| Formulation 1 | 98.49 | 93.93 | 91.76 |
| Formulation 2 | 98.43 | 94.54 | 92.87 |
| Formulation 3 | 98.51 | 94.47 | 92.54 |
| Formulation 4 | 98.72 | 94.69 | 92.76 |
| Formulation 5 | 98.59 | 94.39 | 92.42 |
| Formulation 6 | 98.62 | 93.05 | 90.57 |
| Formulation 7 | 98.59 | 92.88 | 90.19 |
| Formulation 8 | 98.63 | 91.53 | 88.88 |
| Formulation 9 | 98.99 | 92.45 | 89.79 |
| Formulation 10 | 98.59 | 92.35 | 89.67 |
| Formulation 11 | 98.34 | 95.81 | 94.41 |
| Formulation 12 | 98.38 | 95.85 | 94.52 |
| Formulation 13 | 98.37 | 95.93 | 94.47 |
| Formulation 14 | 98.20 | 95.92 | 94.55 |
| Formulation 15 | 98.42 | 96.05 | 94.59 |
| Formulation 16 | 98.77 | 96.31 | 94.99 |
| Formulation 17 | 98.40 | 94.78 | 93.17 |
| Formulation 18 | 98.14 | 94.11 | 92.21 |
| Formulation 19 | 98.19 | 93.98 | 91.89 |
| Formulation 20 | 98.40 | 94.94 | 93.22 |
| Formulation 21 | 98.59 | 94.89 | 93.26 |
| Formulation 22 | 98.61 | 95.37 | 93.58 |
| Formulation 23 | 98.62 | 95.37 | 93.57 |
| Formulation 24 | 98.46 | 94.94 | 93.11 |

TABLE 100
WCX Acidic (%) measurement results (thermal stability)

| | Acidic (%) | | |
|---|---|---|---|
| Formulation | Initial | Week 4 | Week 6 |
| Formulation 1 | 18.64 | 30.20 | 32.53 |
| Formulation 2 | 18.93 | 30.36 | 32.64 |
| Formulation 3 | 18.38 | 29.91 | 32.18 |
| Formulation 4 | 18.33 | 29.29 | 31.94 |
| Formulation 5 | 18.72 | 29.67 | 31.49 |
| Formulation 6 | 18.67 | 24.96 | 24.87 |
| Formulation 7 | 18.73 | 24.73 | 25.00 |
| Formulation 8 | 18.76 | 22.92 | 22.35 |
| Formulation 9 | 18.81 | 23.08 | 22.60 |
| Formulation 10 | 18.43 | 23.54 | 22.64 |
| Formulation 11 | 19.25 | 34.89 | 41.15 |
| Formulation 12 | 19.46 | 35.00 | 41.25 |
| Formulation 13 | 19.32 | 35.41 | 40.57 |
| Formulation 14 | 19.60 | 35.18 | 40.70 |
| Formulation 15 | 19.34 | 38.58 | 42.03 |
| Formulation 16 | 18.97 | 38.44 | 40.62 |
| Formulation 17 | 19.70 | 41.13 | 48.91 |
| Formulation 18 | 19.38 | 40.18 | 49.75 |
| Formulation 19 | 19.93 | 40.81 | 49.68 |
| Formulation 20 | 20.16 | 40.61 | 47.45 |
| Formulation 21 | 19.29 | 40.97 | 47.51 |
| Formulation 22 | 19.61 | 42.72 | 46.96 |
| Formulation 23 | 19.27 | 42.15 | 46.92 |
| Formulation 24 | 19.50 | 41.30 | 48.13 |

TABLE 101
WCX Basic (%) measurement results (thermal stability)

| | Basic (%) | | |
|---|---|---|---|
| Formulation | Initial | Week 4 | Week 6 |
| Formulation 1 | 8.03 | 27.19 | 32.06 |
| Formulation 2 | 7.88 | 26.42 | 31.23 |
| Formulation 3 | 8.23 | 26.99 | 32.10 |
| Formulation 4 | 8.43 | 27.97 | 33.02 |
| Formulation 5 | 8.14 | 27.34 | 32.95 |
| Formulation 6 | 8.21 | 37.09 | 44.83 |
| Formulation 7 | 8.50 | 37.77 | 44.69 |
| Formulation 8 | 8.23 | 41.33 | 48.84 |
| Formulation 9 | 7.91 | 40.63 | 48.55 |
| Formulation 10 | 8.33 | 40.02 | 48.86 |
| Formulation 11 | 7.94 | 12.20 | 13.37 |
| Formulation 12 | 7.32 | 12.27 | 13.04 |
| Formulation 13 | 7.52 | 12.27 | 13.29 |
| Formulation 14 | 7.77 | 12.38 | 13.19 |
| Formulation 15 | 7.45 | 12.16 | 12.94 |
| Formulation 16 | 6.97 | 12.21 | 13.69 |
| Formulation 17 | 7.20 | 8.28 | 9.09 |
| Formulation 18 | 7.39 | 8.19 | 9.10 |
| Formulation 19 | 7.20 | 7.71 | 9.42 |
| Formulation 20 | 7.04 | 8.40 | 9.47 |
| Formulation 21 | 7.16 | 8.34 | 9.34 |
| Formulation 22 | 6.86 | 8.64 | 9.06 |
| Formulation 23 | 7.11 | 8.41 | 9.21 |
| Formulation 24 | 6.96 | 8.45 | 8.51 |

TABLE 102
WCX Main (%) measurement results (thermal stability)

| | Main (%) | | |
|---|---|---|---|
| Formulation | Initial | Week 4 | Week 6 |
| Formulation 1 | 73.33 | 42.61 | 35.41 |
| Formulation 2 | 73.19 | 43.22 | 36.13 |
| Formulation 3 | 73.39 | 43.10 | 35.72 |
| Formulation 4 | 73.24 | 42.74 | 35.04 |
| Formulation 5 | 73.13 | 42.99 | 35.56 |
| Formulation 6 | 73.12 | 37.95 | 30.30 |
| Formulation 7 | 72.77 | 37.50 | 30.31 |
| Formulation 8 | 73.01 | 35.76 | 28.81 |
| Formulation 9 | 73.28 | 36.29 | 28.85 |
| Formulation 10 | 73.24 | 36.44 | 28.51 |
| Formulation 11 | 72.81 | 52.91 | 45.49 |
| Formulation 12 | 73.22 | 52.73 | 45.71 |
| Formulation 13 | 73.16 | 52.32 | 46.14 |
| Formulation 14 | 72.63 | 52.45 | 46.11 |
| Formulation 15 | 73.21 | 49.26 | 45.03 |
| Formulation 16 | 74.06 | 49.35 | 45.69 |
| Formulation 17 | 73.09 | 50.59 | 42.00 |
| Formulation 18 | 73.23 | 51.63 | 41.14 |
| Formulation 19 | 72.87 | 51.48 | 40.91 |
| Formulation 20 | 72.80 | 50.99 | 43.08 |
| Formulation 21 | 73.55 | 50.69 | 43.15 |
| Formulation 22 | 73.52 | 48.64 | 43.99 |
| Formulation 23 | 73.62 | 49.44 | 43.87 |
| Formulation 24 | 73.54 | 50.25 | 43.36 |

TABLE 103
SE-HPLC HMW (%) measurement results (photostability)

| | HMW (%) | | |
|---|---|---|---|
| | | Dark | Light |
| Formulation | Initial | condition | exposure |
| Formulation 1 | 1.12 | 1.14 | 5.58 |
| Formulation 2 | 1.13 | 0.96 | 5.03 |
| Formulation 3 | 1.06 | 1.04 | 4.93 |
| Formulation 4 | 0.89 | 0.83 | 3.71 |
| Formulation 5 | 1.03 | 0.98 | 4.24 |
| Formulation 6 | 0.99 | 0.92 | 3.61 |
| Formulation 7 | 1.02 | 0.93 | 3.14 |
| Formulation 8 | 0.97 | 0.94 | 3.76 |
| Formulation 9 | 0.71 | 0.65 | 3.32 |
| Formulation 10 | 1.00 | 0.94 | 2.88 |
| Formulation 11 | 1.29 | 1.14 | 3.98 |
| Formulation 12 | 1.25 | 1.11 | 4.21 |
| Formulation 13 | 1.27 | 1.16 | 3.76 |
| Formulation 14 | 1.35 | 1.09 | 3.50 |
| Formulation 15 | 1.23 | 1.13 | 3.05 |
| Formulation 16 | 0.86 | 0.74 | 2.59 |
| Formulation 17 | 1.32 | 1.40 | 13.96 |
| Formulation 18 | 1.50 | 1.67 | 14.70 |
| Formulation 19 | 1.50 | 1.62 | 11.37 |
| Formulation 20 | 1.27 | 1.25 | 13.09 |
| Formulation 21 | 1.13 | 1.08 | 3.73 |
| Formulation 22 | 1.06 | 0.92 | 3.59 |
| Formulation 23 | 1.08 | 0.93 | 3.35 |
| Formulation 24 | 1.20 | 1.06 | 3.98 |

TABLE 104
SE-HPLC LMW (%) measurement results (photostability)

| | LMW (%) | | |
|---|---|---|---|
| | | Dark | Light |
| Formulation | Initial | condition | exposure |
| Formulation 1 | 0.39 | 0.45 | 1.06 |
| Formulation 2 | 0.44 | 0.46 | 1.19 |
| Formulation 3 | 0.42 | 0.44 | 1.18 |
| Formulation 4 | 0.39 | 0.42 | 1.16 |
| Formulation 5 | 0.38 | 0.48 | 1.07 |
| Formulation 6 | 0.39 | 0.52 | 1.14 |
| Formulation 7 | 0.39 | 0.50 | 0.97 |
| Formulation 8 | 0.40 | 0.54 | 1.12 |
| Formulation 9 | 0.31 | 0.53 | 1.20 |
| Formulation 10 | 0.41 | 0.50 | 1.01 |
| Formulation 11 | 0.37 | 0.44 | 0.96 |
| Formulation 12 | 0.37 | 0.39 | 1.03 |
| Formulation 13 | 0.36 | 0.42 | 1.00 |
| Formulation 14 | 0.45 | 0.42 | 0.93 |
| Formulation 15 | 0.35 | 0.41 | 0.94 |
| Formulation 16 | 0.37 | 0.40 | 0.99 |
| Formulation 17 | 0.28 | 0.38 | 1.24 |
| Formulation 18 | 0.35 | 0.44 | 1.12 |
| Formulation 19 | 0.31 | 0.37 | 0.92 |
| Formulation 20 | 0.33 | 0.40 | 1.37 |
| Formulation 21 | 0.27 | 0.44 | 0.85 |
| Formulation 22 | 0.33 | 0.42 | 1.10 |
| Formulation 23 | 0.30 | 0.45 | 1.02 |
| Formulation 24 | 0.34 | 0.44 | 0.96 |

TABLE 105
SE-HPLC Monomer (%) measurement results (photostability)

| | Monomer (%) | | |
|---|---|---|---|
| | | Dark | Light |
| Formulation | Initial | condition | exposure |
| Formulation 1 | 98.49 | 98.41 | 93.36 |
| Formulation 2 | 98.43 | 98.58 | 93.78 |
| Formulation 3 | 98.51 | 98.52 | 93.88 |
| Formulation 4 | 98.72 | 98.75 | 95.13 |
| Formulation 5 | 98.59 | 98.54 | 94.69 |
| Formulation 6 | 98.62 | 98.56 | 95.26 |
| Formulation 7 | 98.59 | 98.57 | 95.89 |
| Formulation 8 | 98.63 | 98.52 | 95.12 |
| Formulation 9 | 98.99 | 98.82 | 95.47 |
| Formulation 10 | 98.59 | 98.56 | 96.11 |
| Formulation 11 | 98.34 | 98.42 | 95.06 |
| Formulation 12 | 98.38 | 98.50 | 94.77 |
| Formulation 13 | 98.37 | 98.43 | 95.24 |
| Formulation 14 | 98.20 | 98.49 | 95.57 |
| Formulation 15 | 98.42 | 98.46 | 96.01 |
| Formulation 16 | 98.77 | 98.86 | 96.42 |
| Formulation 17 | 98.40 | 98.22 | 84.80 |
| Formulation 18 | 98.14 | 97.89 | 84.17 |
| Formulation 19 | 98.19 | 98.01 | 87.71 |
| Formulation 20 | 98.40 | 98.36 | 85.54 |
| Formulation 21 | 98.59 | 98.48 | 95.42 |
| Formulation 22 | 98.61 | 98.65 | 95.31 |
| Formulation 23 | 98.62 | 98.62 | 95.63 |
| Formulation 24 | 98.46 | 98.50 | 95.06 |

TABLE 106
WCX Acidic (%) measurement results (photostability)

| | Acidic (%) | | |
|---|---|---|---|
| | | Dark | Light |
| Formulation | Initial | condition | exposure |
| Formulation 1 | 18.64 | 18.52 | 20.25 |
| Formulation 2 | 18.93 | 19.22 | 20.72 |
| Formulation 3 | 18.38 | 18.85 | 20.22 |
| Formulation 4 | 18.33 | 18.66 | 20.69 |
| Formulation 5 | 18.72 | 18.53 | 20.24 |
| Formulation 6 | 18.67 | 18.17 | 19.78 |
| Formulation 7 | 18.73 | 18.36 | 18.39 |
| Formulation 8 | 18.76 | 18.17 | 19.14 |
| Formulation 9 | 18.81 | 17.66 | 19.52 |
| Formulation 10 | 18.43 | 18.05 | 19.76 |
| Formulation 11 | 19.25 | 19.48 | 31.53 |
| Formulation 12 | 19.46 | 19.33 | 33.51 |
| Formulation 13 | 19.32 | 18.94 | 31.40 |
| Formulation 14 | 19.60 | 19.13 | 30.96 |
| Formulation 15 | 19.34 | 19.02 | 31.79 |
| Formulation 16 | 18.97 | 18.67 | 32.23 |
| Formulation 17 | 19.70 | 20.64 | 50.61 |
| Formulation 18 | 19.38 | 20.58 | 45.10 |
| Formulation 19 | 19.93 | 20.29 | 40.27 |
| Formulation 20 | 20.16 | 20.54 | 51.78 |
| Formulation 21 | 19.29 | 20.02 | 36.43 |
| Formulation 22 | 19.61 | 19.57 | 40.53 |
| Formulation 23 | 19.27 | 20.00 | 38.78 |
| Formulation 24 | 19.50 | 20.53 | 38.15 |

TABLE 107
WCX Basic (%) measurement results (photostability)

| | Basic (%) | | |
|---|---|---|---|
| | | Dark | Light |
| Formulation | Initial | condition | exposure |
| Formulation 1 | 8.03 | 10.67 | 46.02 |
| Formulation 2 | 7.88 | 10.30 | 49.01 |
| Formulation 3 | 8.23 | 11.08 | 44.99 |
| Formulation 4 | 8.43 | 10.93 | 41.38 |
| Formulation 5 | 8.14 | 10.96 | 41.41 |
| Formulation 6 | 8.21 | 11.55 | 40.12 |
| Formulation 7 | 8.50 | 10.89 | 36.78 |
| Formulation 8 | 8.23 | 11.25 | 36.95 |
| Formulation 9 | 7.91 | 11.38 | 38.87 |
| Formulation 10 | 8.33 | 11.57 | 31.62 |
| Formulation 11 | 7.94 | 8.62 | 19.08 |
| Formulation 12 | 7.32 | 8.10 | 20.30 |
| Formulation 13 | 7.52 | 8.69 | 18.49 |
| Formulation 14 | 7.77 | 8.24 | 18.92 |
| Formulation 15 | 7.45 | 8.19 | 16.92 |
| Formulation 16 | 6.97 | 7.95 | 16.57 |
| Formulation 17 | 7.20 | 7.70 | 21.93 |
| Formulation 18 | 7.39 | 7.72 | 22.42 |
| Formulation 19 | 7.20 | 8.11 | 18.61 |
| Formulation 20 | 7.04 | 7.72 | 21.66 |
| Formulation 21 | 7.16 | 7.72 | 12.07 |
| Formulation 22 | 6.86 | 8.13 | 11.66 |
| Formulation 23 | 7.11 | 7.63 | 12.12 |
| Formulation 24 | 6.96 | 7.64 | 12.48 |

TABLE 108
WCX Main (%) measurement results (photostability)

| | Main (%) | | |
|---|---|---|---|
| | | Dark | Light |
| Formulation | Initial | condition | exposure |
| Formulation 1 | 73.33 | 70.81 | 33.73 |
| Formulation 2 | 73.19 | 70.49 | 30.26 |
| Formulation 3 | 73.39 | 70.07 | 34.79 |
| Formulation 4 | 73.24 | 70.41 | 37.93 |
| Formulation 5 | 73.13 | 70.51 | 38.35 |
| Formulation 6 | 73.12 | 70.28 | 40.10 |
| Formulation 7 | 72.77 | 70.75 | 44.84 |
| Formulation 8 | 73.01 | 70.58 | 43.91 |
| Formulation 9 | 73.28 | 70.95 | 41.62 |
| Formulation 10 | 73.24 | 70.38 | 48.63 |
| Formulation 11 | 72.81 | 71.90 | 49.39 |
| Formulation 12 | 73.22 | 72.57 | 46.18 |
| Formulation 13 | 73.16 | 72.36 | 50.11 |
| Formulation 14 | 72.63 | 72.63 | 50.12 |
| Formulation 15 | 73.21 | 72.78 | 51.29 |
| Formulation 16 | 74.06 | 73.37 | 51.20 |
| Formulation 17 | 73.09 | 71.66 | 27.46 |
| Formulation 18 | 73.23 | 71.70 | 32.47 |
| Formulation 19 | 72.87 | 71.60 | 41.12 |
| Formulation 20 | 72.80 | 71.75 | 26.55 |
| Formulation 21 | 73.55 | 72.27 | 51.49 |
| Formulation 22 | 73.52 | 72.30 | 47.80 |
| Formulation 23 | 73.62 | 72.37 | 49.10 |
| Formulation 24 | 73.54 | 71.83 | 49.37 |

TABLE 109
HIC Hydrophobic (%) measurement results (photostability)

Hydrophobic (%)

| Formulation | Initial | Dark condition | Light exposure |
|---|---|---|---|
| Formulation 1 | 4.93 | 7.06 | 57.01 |
| Formulation 2 | 5.08 | 7.59 | 63.87 |
| Formulation 3 | 5.03 | 8.36 | 56.34 |
| Formulation 4 | 5.03 | 7.96 | 51.25 |
| Formulation 5 | 5.19 | 6.67 | 50.45 |
| Formulation 6 | 5.47 | 7.74 | 48.52 |
| Formulation 7 | 5.56 | 6.82 | 42.43 |
| Formulation 8 | 5.51 | 6.87 | 44.10 |
| Formulation 9 | 5.47 | 6.60 | 48.04 |
| Formulation 10 | 5.52 | 6.83 | 35.47 |
| Formulation 11 | 5.56 | 6.63 | 31.63 |
| Formulation 12 | 5.64 | 6.72 | 36.95 |
| Formulation 13 | 5.77 | 6.74 | 30.26 |
| Formulation 14 | 5.61 | 6.76 | 30.65 |
| Formulation 15 | 5.70 | 6.90 | 32.40 |
| Formulation 16 | 5.56 | 6.74 | 34.34 |
| Formulation 17 | 5.79 | 7.27 | 47.95 |
| Formulation 18 | 5.84 | 7.09 | 38.24 |
| Formulation 19 | 5.90 | 7.17 | 28.93 |
| Formulation 20 | 5.72 | 7.18 | 50.37 |
| Formulation 21 | 5.97 | 7.06 | 25.44 |
| Formulation 22 | 5.88 | 7.12 | 31.11 |
| Formulation 23 | 5.87 | 7.01 | 30.20 |
| Formulation 24 | 6.06 | 7.34 | 27.64 |

TABLE 110
HIC Hydrophilic (%) measurement results (photostability)

Hydrophilic (%)

| Formulation | Initial | Dark condition | Light exposure |
|---|---|---|---|
| Formulation 1 | 2.67 | 4.69 | 6.96 |
| Formulation 2 | 2.84 | 4.62 | 7.65 |
| Formulation 3 | 2.84 | 4.66 | 6.89 |
| Formulation 4 | 2.89 | 4.30 | 6.25 |
| Formulation 5 | 2.91 | 4.55 | 6.72 |
| Formulation 6 | 3.06 | 4.31 | 6.10 |
| Formulation 7 | 3.21 | 4.52 | 6.00 |
| Formulation 8 | 3.20 | 4.50 | 6.30 |
| Formulation 9 | 3.15 | 4.32 | 5.83 |
| Formulation 10 | 3.31 | 4.53 | 6.05 |
| Formulation 11 | 3.70 | 4.75 | 6.96 |
| Formulation 12 | 3.77 | 4.71 | 6.92 |
| Formulation 13 | 3.85 | 4.75 | 6.77 |
| Formulation 14 | 3.89 | 4.81 | 6.67 |
| Formulation 15 | 3.98 | 4.63 | 6.22 |
| Formulation 16 | 3.86 | 4.49 | 5.83 |
| Formulation 17 | 4.24 | 4.97 | 13.21 |
| Formulation 18 | 4.42 | 5.06 | 12.37 |
| Formulation 19 | 4.38 | 5.09 | 11.75 |
| Formulation 20 | 4.30 | 4.85 | 12.74 |
| Formulation 21 | 4.24 | 4.62 | 6.93 |
| Formulation 22 | 4.22 | 4.68 | 6.71 |
| Formulation 23 | 4.27 | 4.61 | 6.53 |
| Formulation 24 | 4.42 | 4.77 | 7.01 |

TABLE 111
HIC Main (%) measurement results (photostability)

Main (%)

| Formulation | Initial | Dark condition | Light exposure |
|---|---|---|---|
| Formulation 1 | 92.40 | 88.25 | 36.03 |
| Formulation 2 | 92.08 | 87.78 | 28.48 |
| Formulation 3 | 92.13 | 86.97 | 36.77 |
| Formulation 4 | 92.08 | 87.74 | 42.50 |
| Formulation 5 | 91.90 | 88.78 | 42.83 |
| Formulation 6 | 91.47 | 87.95 | 45.39 |
| Formulation 7 | 91.23 | 88.66 | 51.58 |
| Formulation 8 | 91.29 | 88.63 | 49.59 |
| Formulation 9 | 91.38 | 89.08 | 46.13 |
| Formulation 10 | 91.17 | 88.65 | 58.48 |
| Formulation 11 | 90.74 | 88.62 | 61.40 |
| Formulation 12 | 90.59 | 88.57 | 56.14 |
| Formulation 13 | 90.39 | 88.52 | 62.97 |
| Formulation 14 | 90.50 | 88.42 | 62.68 |
| Formulation 15 | 90.32 | 88.48 | 61.38 |
| Formulation 16 | 90.58 | 88.77 | 59.83 |
| Formulation 17 | 89.97 | 87.77 | 38.84 |
| Formulation 18 | 89.75 | 87.85 | 49.40 |
| Formulation 19 | 89.71 | 87.74 | 59.33 |
| Formulation 20 | 89.98 | 87.98 | 36.88 |
| Formulation 21 | 89.78 | 88.32 | 67.63 |
| Formulation 22 | 89.89 | 88.20 | 62.18 |
| Formulation 23 | 89.86 | 88.38 | 63.27 |
| Formulation 24 | 89.52 | 87.89 | 65.35 |

TABLE 112
SE-HPLC HMW (%) measurement results (freeze-thaw stability)

HMW (%)

| Formulation | Initial | 5 freeze-thaw cycles |
|---|---|---|
| Formulation 1 | 1.12 | 1.19 |
| Formulation 2 | 1.13 | 1.14 |
| Formulation 3 | 1.06 | 1.12 |
| Formulation 4 | 0.89 | 0.90 |
| Formulation 5 | 1.03 | 1.11 |
| Formulation 6 | 0.99 | 1.08 |
| Formulation 7 | 1.02 | 1.13 |
| Formulation 8 | 0.97 | 1.08 |
| Formulation 9 | 0.71 | 0.74 |
| Formulation 10 | 1.00 | 1.04 |
| Formulation 11 | 1.29 | 1.47 |
| Formulation 12 | 1.25 | 1.37 |
| Formulation 13 | 1.27 | 1.33 |
| Formulation 14 | 1.35 | 1.30 |
| Formulation 15 | 1.23 | 1.34 |
| Formulation 16 | 0.86 | 0.91 |
| Formulation 17 | 1.32 | 1.29 |
| Formulation 18 | 1.50 | 1.54 |
| Formulation 19 | 1.50 | 1.42 |
| Formulation 20 | 1.27 | 1.29 |
| Formulation 21 | 1.13 | 1.23 |
| Formulation 22 | 1.06 | 1.13 |
| Formulation 23 | 1.08 | 1.14 |
| Formulation 24 | 1.20 | 1.21 |

TABLE 113
SE-HPLC LMW (%) measurement results (freeze-thaw stability)

LMW (%)

| Formulation | Initial | 5 freeze-thaw cycles |
|---|---|---|
| Formulation 1 | 0.39 | 0.41 |
| Formulation 2 | 0.44 | 0.43 |
| Formulation 3 | 0.42 | 0.39 |
| Formulation 4 | 0.39 | 0.37 |
| Formulation 5 | 0.38 | 0.43 |
| Formulation 6 | 0.39 | 0.39 |
| Formulation 7 | 0.39 | 0.45 |
| Formulation 8 | 0.40 | 0.41 |
| Formulation 9 | 0.31 | 0.40 |
| Formulation 10 | 0.41 | 0.41 |
| Formulation 11 | 0.37 | 0.36 |
| Formulation 12 | 0.37 | 0.37 |
| Formulation 13 | 0.36 | 0.34 |
| Formulation 14 | 0.45 | 0.36 |
| Formulation 15 | 0.35 | 0.40 |
| Formulation 16 | 0.37 | 0.32 |
| Formulation 17 | 0.28 | 0.29 |
| Formulation 18 | 0.35 | 0.31 |
| Formulation 19 | 0.31 | 0.28 |
| Formulation 20 | 0.33 | 0.39 |
| Formulation 21 | 0.27 | 0.30 |
| Formulation 22 | 0.33 | 0.33 |
| Formulation 23 | 0.30 | 0.31 |
| Formulation 24 | 0.34 | 0.35 |

TABLE 114
SE-HPLC Monomer (%) measurement
results (freeze-thaw stability)

Monomer (%)

| Formulation | Initial | 5 freeze-thaw cycles |
|---|---|---|
| Formulation 1 | 98.49 | 98.39 |
| Formulation 2 | 98.43 | 98.43 |
| Formulation 3 | 98.51 | 98.50 |
| Formulation 4 | 98.72 | 98.73 |
| Formulation 5 | 98.59 | 98.46 |
| Formulation 6 | 98.62 | 98.53 |
| Formulation 7 | 98.59 | 98.42 |
| Formulation 8 | 98.63 | 98.51 |
| Formulation 9 | 98.99 | 98.86 |
| Formulation 10 | 98.59 | 98.54 |
| Formulation 11 | 98.34 | 98.17 |
| Formulation 12 | 98.38 | 98.26 |
| Formulation 13 | 98.37 | 98.33 |
| Formulation 14 | 98.20 | 98.33 |
| Formulation 15 | 98.42 | 98.26 |
| Formulation 16 | 98.77 | 98.77 |
| Formulation 17 | 98.40 | 98.42 |
| Formulation 18 | 98.14 | 98.15 |
| Formulation 19 | 98.19 | 98.30 |
| Formulation 20 | 98.40 | 98.32 |
| Formulation 21 | 98.59 | 98.48 |
| Formulation 22 | 98.61 | 98.54 |
| Formulation 23 | 98.62 | 98.55 |
| Formulation 24 | 98.46 | 98.44 |

TABLE 115
WCX Acidic (%) measurement results (freeze-thaw stability)

Acidic (%)

| Formulation | Initial | 5 freeze-thaw cycles |
|---|---|---|
| Formulation 1 | 18.64 | 18.89 |
| Formulation 2 | 18.93 | 19.07 |
| Formulation 3 | 18.38 | 19.11 |
| Formulation 4 | 18.33 | 18.70 |
| Formulation 5 | 18.72 | 19.10 |
| Formulation 6 | 18.67 | 19.12 |
| Formulation 7 | 18.73 | 18.98 |
| Formulation 8 | 18.76 | 18.76 |
| Formulation 9 | 18.81 | 18.58 |
| Formulation 10 | 18.43 | 18.75 |
| Formulation 11 | 19.25 | 18.79 |
| Formulation 12 | 19.46 | 18.91 |
| Formulation 13 | 19.32 | 18.95 |
| Formulation 14 | 19.60 | 19.28 |
| Formulation 15 | 19.34 | 18.69 |
| Formulation 16 | 18.97 | 18.94 |
| Formulation 17 | 19.70 | 19.51 |
| Formulation 18 | 19.38 | 19.39 |
| Formulation 19 | 19.93 | 19.60 |
| Formulation 20 | 20.16 | 19.89 |
| Formulation 21 | 19.29 | 19.03 |
| Formulation 22 | 19.61 | 19.39 |
| Formulation 23 | 19.27 | 19.04 |
| Formulation 24 | 19.50 | 19.00 |

TABLE 116
WCX Basic (%) measurement results (freeze-thaw stability)

Basic (%)

| Formulation | Initial | 5 freeze-thaw cycles |
|---|---|---|
| Formulation 1 | 8.03 | 7.86 |
| Formulation 2 | 7.88 | 7.73 |
| Formulation 3 | 8.23 | 7.79 |
| Formulation 4 | 8.43 | 7.75 |
| Formulation 5 | 8.14 | 7.74 |
| Formulation 6 | 8.21 | 7.49 |
| Formulation 7 | 8.50 | 7.80 |
| Formulation 8 | 8.23 | 7.95 |
| Formulation 9 | 7.91 | 7.65 |
| Formulation 10 | 8.33 | 7.95 |
| Formulation 11 | 7.94 | 7.89 |
| Formulation 12 | 7.32 | 7.70 |
| Formulation 13 | 7.52 | 7.63 |
| Formulation 14 | 7.77 | 7.36 |
| Formulation 15 | 7.45 | 7.68 |
| Formulation 16 | 6.97 | 7.06 |
| Formulation 17 | 7.20 | 7.36 |
| Formulation 18 | 7.39 | 7.39 |
| Formulation 19 | 7.20 | 7.26 |
| Formulation 20 | 7.04 | 7.01 |
| Formulation 21 | 7.16 | 7.35 |
| Formulation 22 | 6.86 | 6.92 |
| Formulation 23 | 7.11 | 7.59 |
| Formulation 24 | 6.96 | 7.64 |

TABLE 117
WCX Main (%) measurement results (freeze-thaw stability)

Main (%)

| Formulation | Initial | 5 freeze-thaw cycles |
|---|---|---|
| Formulation 1 | 73.33 | 73.25 |
| Formulation 2 | 73.19 | 73.20 |
| Formulation 3 | 73.39 | 73.10 |
| Formulation 4 | 73.24 | 73.55 |
| Formulation 5 | 73.13 | 73.16 |
| Formulation 6 | 73.12 | 73.39 |
| Formulation 7 | 72.77 | 73.22 |
| Formulation 8 | 73.01 | 73.29 |
| Formulation 9 | 73.28 | 73.77 |
| Formulation 10 | 73.24 | 73.30 |
| Formulation 11 | 72.81 | 73.32 |
| Formulation 12 | 73.22 | 73.38 |
| Formulation 13 | 73.16 | 73.42 |
| Formulation 14 | 72.63 | 73.36 |
| Formulation 15 | 73.21 | 73.62 |
| Formulation 16 | 74.06 | 74.00 |
| Formulation 17 | 73.09 | 73.13 |
| Formulation 18 | 73.23 | 73.22 |
| Formulation 19 | 72.87 | 73.14 |
| Formulation 20 | 72.80 | 73.11 |
| Formulation 21 | 73.55 | 73.62 |
| Formulation 22 | 73.52 | 73.69 |
| Formulation 23 | 73.62 | 73.37 |
| Formulation 24 | 73.54 | 73.36 |

TABLE 118
SE-HPLC HMW (%) measurement results (agitation stability)

HMW (%)

| Formulation | 0 rpm | 400 rpm |
|---|---|---|
| Formulation 1 | 1.09 | 1.08 |
| Formulation 2 | 1.05 | 1.03 |
| Formulation 3 | 1.05 | 1.02 |
| Formulation 4 | 0.85 | 0.82 |
| Formulation 5 | 1.08 | 0.98 |
| Formulation 6 | 0.96 | 0.92 |
| Formulation 7 | 0.96 | 1.00 |
| Formulation 8 | 1.04 | 1.06 |
| Formulation 9 | 0.67 | 0.74 |
| Formulation 10 | 1.01 | 1.04 |
| Formulation 11 | 1.25 | 1.28 |
| Formulation 12 | 1.20 | 1.30 |
| Formulation 13 | 1.25 | 1.32 |
| Formulation 14 | 1.24 | 1.21 |
| Formulation 15 | 1.17 | 1.19 |
| Formulation 16 | 0.78 | 0.77 |
| Formulation 17 | 1.37 | 1.40 |
| Formulation 18 | 1.58 | 1.59 |
| Formulation 19 | 1.58 | 1.64 |
| Formulation 20 | 1.32 | 1.24 |
| Formulation 21 | 1.10 | 1.27 |
| Formulation 22 | 0.99 | 1.01 |
| Formulation 23 | 0.97 | 1.08 |
| Formulation 24 | 1.12 | 1.16 |

TABLE 119
SE-HPLC LMW (%) measurement results (agitation stability)

LMW (%)

| Formulation | 0 rpm | 400 rpm |
|---|---|---|
| Formulation 1 | 0.46 | 0.42 |
| Formulation 2 | 0.41 | 0.40 |
| Formulation 3 | 0.42 | 0.45 |
| Formulation 4 | 0.43 | 0.47 |
| Formulation 5 | 0.44 | 0.42 |
| Formulation 6 | 0.45 | 0.43 |
| Formulation 7 | 0.43 | 0.46 |
| Formulation 8 | 0.51 | 0.46 |
| Formulation 9 | 0.43 | 0.45 |
| Formulation 10 | 0.42 | 0.43 |
| Formulation 11 | 0.38 | 0.38 |
| Formulation 12 | 0.41 | 0.37 |
| Formulation 13 | 0.38 | 0.38 |
| Formulation 14 | 0.35 | 0.38 |
| Formulation 15 | 0.40 | 0.36 |
| Formulation 16 | 0.37 | 0.36 |
| Formulation 17 | 0.32 | 0.35 |
| Formulation 18 | 0.35 | 0.35 |
| Formulation 19 | 0.35 | 0.37 |
| Formulation 20 | 0.37 | 0.38 |
| Formulation 21 | 0.37 | 0.39 |
| Formulation 22 | 0.38 | 0.35 |
| Formulation 23 | 0.36 | 0.30 |
| Formulation 24 | 0.31 | 0.37 |

TABLE 120
SE-HPLC Monomer (%) measurement results (agitation stability)

Monomer (%)

| Formulation | 0 rpm | 400 rpm |
|---|---|---|
| Formulation 1 | 98.45 | 98.50 |
| Formulation 2 | 98.54 | 98.57 |
| Formulation 3 | 98.53 | 98.54 |
| Formulation 4 | 98.72 | 98.71 |
| Formulation 5 | 98.47 | 98.60 |
| Formulation 6 | 98.59 | 98.65 |
| Formulation 7 | 98.62 | 98.54 |
| Formulation 8 | 98.45 | 98.48 |
| Formulation 9 | 98.91 | 98.81 |
| Formulation 10 | 98.57 | 98.53 |
| Formulation 11 | 98.38 | 98.34 |
| Formulation 12 | 98.40 | 98.33 |
| Formulation 13 | 98.37 | 98.31 |
| Formulation 14 | 98.42 | 98.41 |
| Formulation 15 | 98.43 | 98.45 |
| Formulation 16 | 98.86 | 98.87 |
| Formulation 17 | 98.31 | 98.25 |
| Formulation 18 | 98.08 | 98.06 |
| Formulation 19 | 98.08 | 97.99 |
| Formulation 20 | 98.30 | 98.38 |
| Formulation 21 | 98.52 | 98.34 |
| Formulation 22 | 98.63 | 98.64 |
| Formulation 23 | 98.67 | 98.62 |
| Formulation 24 | 98.57 | 98.47 |

TABLE 121
WCX Acidic (%) measurement results (agitation stability)

Acidic (%)

| Formulation | 0 rpm | 400 rpm |
|---|---|---|
| Formulation 1 | 19.37 | 19.30 |
| Formulation 2 | 18.99 | 19.38 |
| Formulation 3 | 19.05 | 19.29 |
| Formulation 4 | 19.28 | 19.39 |
| Formulation 5 | 19.05 | 19.33 |
| Formulation 6 | 19.03 | 19.10 |
| Formulation 7 | 18.68 | 19.33 |
| Formulation 8 | 18.28 | 19.28 |
| Formulation 9 | 18.79 | 19.05 |
| Formulation 10 | 18.46 | 19.23 |
| Formulation 11 | 19.55 | 19.39 |
| Formulation 12 | 19.35 | 19.84 |
| Formulation 13 | 19.62 | 19.95 |
| Formulation 14 | 19.23 | 19.87 |
| Formulation 15 | 19.35 | 19.64 |
| Formulation 16 | 19.16 | 19.91 |
| Formulation 17 | 20.27 | 20.74 |
| Formulation 18 | 19.89 | 20.41 |
| Formulation 19 | 20.16 | 20.43 |
| Formulation 20 | 20.27 | 20.68 |
| Formulation 21 | 19.89 | 19.95 |
| Formulation 22 | 20.12 | 20.50 |
| Formulation 23 | 19.75 | 20.26 |
| Formulation 24 | 19.24 | 20.42 |

TABLE 122
WCX Basic (%) measurement results (agitation stability)

Basic (%)

| Formulation | 0 rpm | 400 rpm |
|---|---|---|
| Formulation 1 | 8.51 | 8.39 |
| Formulation 2 | 8.44 | 8.43 |
| Formulation 3 | 8.51 | 8.55 |
| Formulation 4 | 8.25 | 8.26 |
| Formulation 5 | 8.67 | 8.49 |
| Formulation 6 | 8.84 | 9.00 |
| Formulation 7 | 9.12 | 8.91 |
| Formulation 8 | 9.28 | 9.07 |
| Formulation 9 | 8.72 | 8.78 |
| Formulation 10 | 9.32 | 9.18 |
| Formulation 11 | 7.66 | 7.49 |
| Formulation 12 | 7.62 | 7.47 |
| Formulation 13 | 7.39 | 7.51 |
| Formulation 14 | 7.49 | 7.60 |
| Formulation 15 | 7.37 | 7.40 |
| Formulation 16 | 7.36 | 7.07 |
| Formulation 17 | 7.08 | 7.09 |
| Formulation 18 | 7.24 | 7.13 |
| Formulation 19 | 7.20 | 7.27 |
| Formulation 20 | 6.98 | 6.97 |
| Formulation 21 | 7.31 | 7.06 |
| Formulation 22 | 6.85 | 6.91 |
| Formulation 23 | 7.04 | 6.82 |
| Formulation 24 | 7.29 | 7.00 |

TABLE 123
WCX Main (%) measurement results (agitation stability)

Main (%)

| Formulation | 0 rpm | 400 rpm |
|---|---|---|
| Formulation 1 | 72.12 | 72.31 |
| Formulation 2 | 72.57 | 72.19 |
| Formulation 3 | 72.45 | 72.15 |
| Formulation 4 | 72.47 | 72.36 |
| Formulation 5 | 72.28 | 72.18 |
| Formulation 6 | 72.13 | 71.90 |
| Formulation 7 | 72.20 | 71.75 |
| Formulation 8 | 72.44 | 71.65 |
| Formulation 9 | 72.49 | 72.17 |
| Formulation 10 | 72.22 | 71.59 |
| Formulation 11 | 72.79 | 73.12 |
| Formulation 12 | 73.03 | 72.69 |
| Formulation 13 | 72.99 | 72.54 |
| Formulation 14 | 73.28 | 72.53 |
| Formulation 15 | 73.28 | 72.95 |
| Formulation 16 | 73.48 | 73.02 |
| Formulation 17 | 72.65 | 72.18 |
| Formulation 18 | 72.86 | 72.46 |
| Formulation 19 | 72.64 | 72.30 |
| Formulation 20 | 72.75 | 72.35 |
| Formulation 21 | 72.80 | 72.99 |
| Formulation 22 | 73.03 | 72.60 |
| Formulation 23 | 73.21 | 72.93 |
| Formulation 24 | 73.47 | 72.58 |

### DISCUSSION OF RESULTS

The appropriate concentration range of each ingredient of the proline-containing formulation was confirmed through the DoE statistical analysis on the test results of the thermal stability, freeze-thaw stability, agitation stability, and photostability stability.

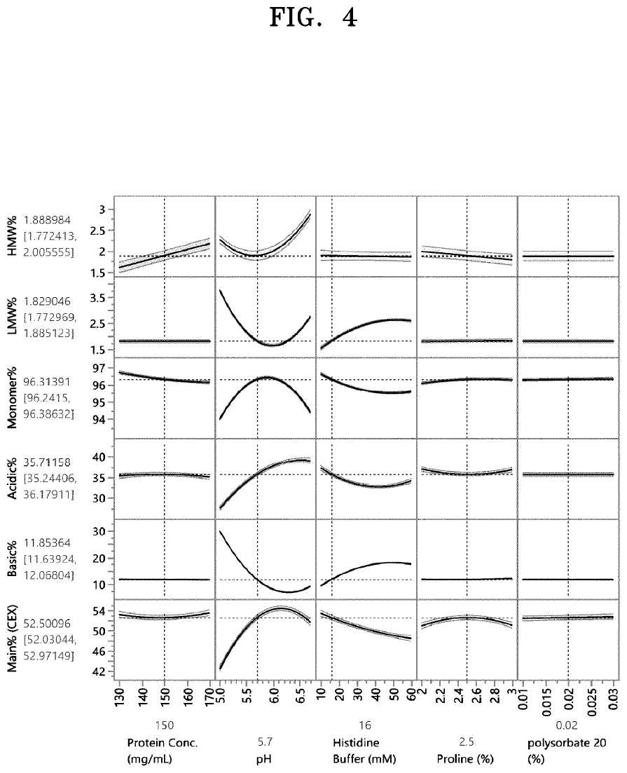

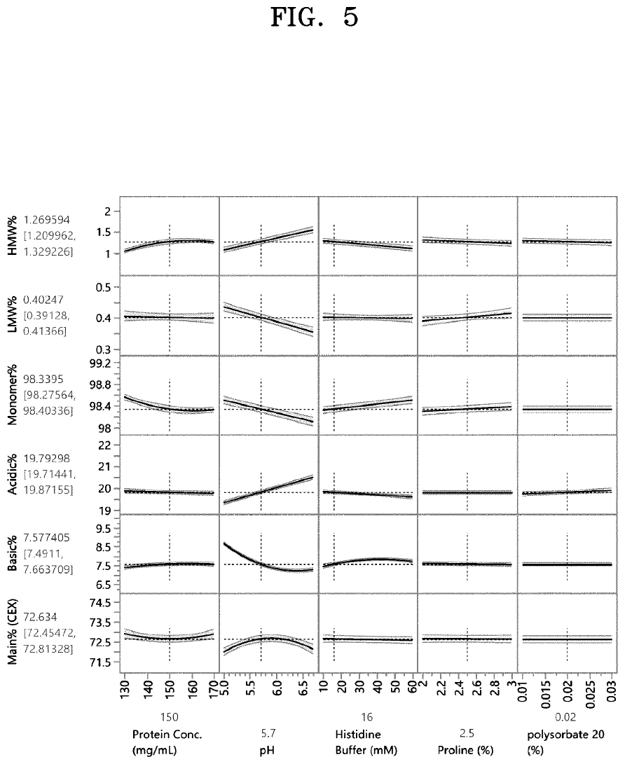

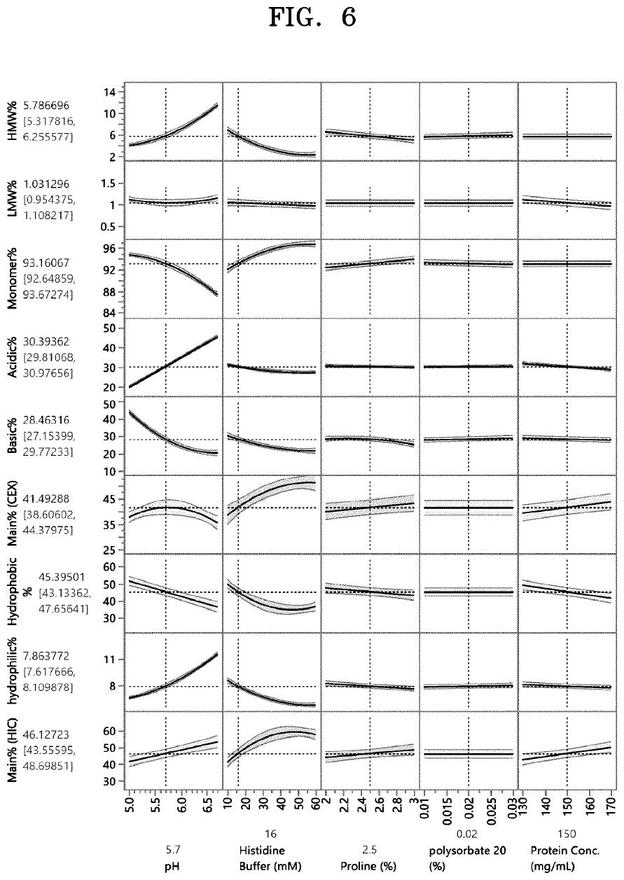

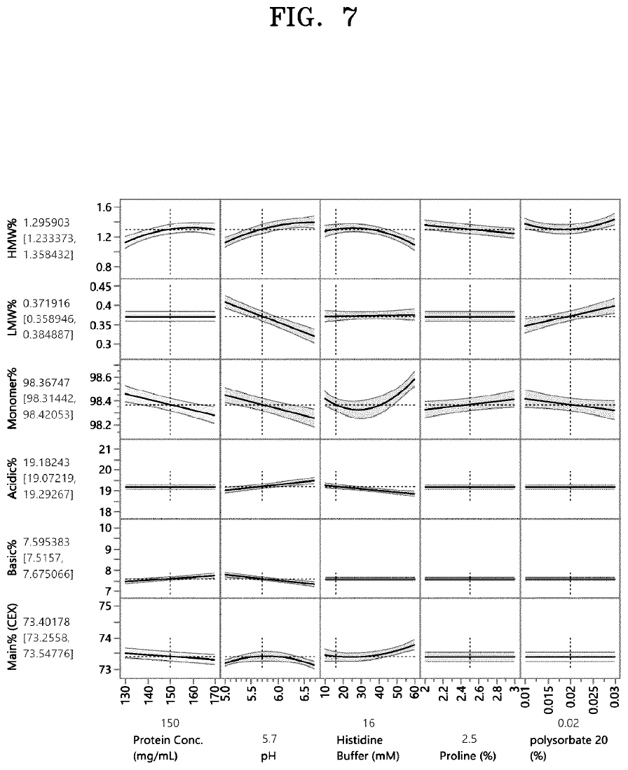

The graphs obtained by performing stability modeling based on the DoE statistical analysis on the thermal stability test results (SE-HPLC and WCX results), the agitation stability test results (SE-HPLC and WCX results), the photostability test results (SE-HPLC, WCX, and HIC results), and the freeze-thaw stability results (SE-HPLC and WCX results) are shown in FIGS. 4, 5, 6, and 7, respectively.

According to the results of FIGS. 4, 5, 6, and 7, 150 mg/mL risankizumab was confirmed to have optimal stability at pH 5.7 and with 16 mM histidine, proline stabilizer at a concentration of 2.5 wt %, and polysorbate 20 at a concentration of 0.02 wt %, in terms of stability, agitation stability, light stability, and freeze-thaw stability.

1. An aqueous pharmaceutical composition comprising:
(a) risankizumab or an antigen-binding fragment thereof; and
(b) a stabilizer,
wherein the aqueous pharmaceutical composition does not comprise a polyol.

2. The aqueous pharmaceutical composition of claim 1, wherein the stabilizer comprises an amino acid or a pharmaceutically acceptable salt thereof, or sodium chloride.

3. An aqueous pharmaceutical composition comprising:
(a) risankizumab or an antigen-binding fragment thereof; and
(b) an amino acid or a pharmaceutically acceptable salt thereof,
wherein (c) the aqueous pharmaceutical composition has a pH of 5.0 to 7.0.

4. The aqueous pharmaceutical composition of claim 3, not comprising a polyol.

5. The aqueous pharmaceutical composition of claim 2, wherein the amino acid comprises lysine, arginine, glycine, proline, histidine, phenylalanine, tyrosine, tryptophan, a pharmaceutically acceptable salt of the foregoing, or a mixture of the foregoing.

6. The aqueous pharmaceutical composition of claim 1, wherein the polyol comprises sorbitol, sucrose, trehalose, mannose, maltose, mannitol, or a mixture of the foregoing.

7. The aqueous pharmaceutical composition of claim 1, further comprising a surfactant.

8. The aqueous pharmaceutical composition of claim 1, not comprising a surfactant.

9. The aqueous pharmaceutical composition of claim 7, wherein the surfactant comprises polysorbate, poloxamer, a sorbitan ester of another fatty acid, or a mixture of the foregoing.

10. The aqueous pharmaceutical composition of claim 9, wherein the polysorbate comprises polysorbate 20, polysorbate 80, or a mixture of the foregoing.

11. The aqueous pharmaceutical composition of claim 1, having a pH of 5.0 to 7.0.

12. The aqueous pharmaceutical composition of claim 1, further comprising a buffer.

13. The pharmaceutical composition of claim 12, wherein the buffer comprises acetate, succinate, citrate, glutamate, glycine, lactate, maleate, phosphate, tartrate, histidine, or any combination of the foregoing.

14. The pharmaceutical composition of claim 1, wherein a concentration of the risankizumab or the antigen-binding fragment thereof is 9 mg/ml to 170 mg/ml.

15. The pharmaceutical composition of claim 1, wherein the pharmaceutical composition is for subcutaneous injection, intramuscular injection, or intravenous injection.

16. The pharmaceutical composition of claim 1, wherein the pharmaceutical composition is for treating autoimmune diseases, cancer, psoriasis, psoriatic arthritis, inflammatory bowel disease, multiple sclerosis, rheumatoid arthritis, ankylosing spondylitis, asthma, or chronic obstructive pulmonary disease (COPD).
